SEQUENCE LISTING (799 sequences)

SEQ ID NO: 1 - protein, 15 aa, Artificial Sequence
  Peptide M_1
MADSNGTITVEELKK

SEQ ID NO: 2 - protein, 15 aa, Artificial Sequence
  Peptide M_2
GTITVEELKKLLEQW

SEQ ID NO: 3 - protein, 15 aa, Artificial Sequence
  Peptide M_3
EELKKLLEQWNLVIG

SEQ ID NO: 4 - protein, 15 aa, Artificial Sequence
  Peptide M_4
LLEQWNLVIGFLFLT

SEQ ID NO: 5 - protein, 15 aa, Artificial Sequence
  Peptide M_5
NLVIGFLFLTWICLL

SEQ ID NO: 6 - protein, 15 aa, Artificial Sequence
  Peptide M_6
FLFLTWICLLQFAYA

SEQ ID NO: 7 - protein, 15 aa, Artificial Sequence
  Peptide M_7
WICLLQFAYANRNRF

SEQ ID NO: 8 - protein, 15 aa, Artificial Sequence
  Peptide M_8
QFAYANRNRFLYIIK

SEQ ID NO: 9 - protein, 15 aa, Artificial Sequence
  Peptide M_9
NRNRFLYIIKLIFLW

SEQ ID NO: 10 - protein, 15 aa, Artificial Sequence
  Peptide M_10
LYIIKLIFLWLLWPV

SEQ ID NO: 11 - protein, 15 aa, Artificial Sequence
  Peptide M_11
LIFLWLLWPVTLACF

SEQ ID NO: 12 - protein, 15 aa, Artificial Sequence
  Peptide M_12
LLWPVTLACFVLAAV

SEQ ID NO: 13 - protein, 15 aa, Artificial Sequence
  Peptide M_13
TLACFVLAAVYRINW

SEQ ID NO: 14 - protein, 15 aa, Artificial Sequence
  Peptide M_14
VLAAVYRINWITGGI

SEQ ID NO: 15 - protein, 15 aa, Artificial Sequence
  Peptide M_15
YRINWITGGIAIAMA

SEQ ID NO: 16 - protein, 15 aa, Artificial Sequence
  Peptide M_16
ITGGIAIAMACLVGL

SEQ ID NO: 17 - protein, 15 aa, Artificial Sequence
  Peptide M_17
AIAMACLVGLMWLSY

SEQ ID NO: 18 - protein, 15 aa, Artificial Sequence
  Peptide M_18
CLVGLMWLSYFIASF

SEQ ID NO: 19 - protein, 15 aa, Artificial Sequence
  Peptide M_19
MWLSYFIASFRLFAR

SEQ ID NO: 20 - protein, 15 aa, Artificial Sequence
  Peptide M_20
FIASFRLFARTRSMW

SEQ ID NO: 21 - protein, 15 aa, Artificial Sequence
  Peptide M_21
RLFARTRSMWSFNPE

SEQ ID NO: 22 - protein, 15 aa, Artificial Sequence
  Peptide M_22
TRSMWSFNPETNILL

SEQ ID NO: 23 - protein, 15 aa, Artificial Sequence
  Peptide M_23
SFNPETNILLNVPLH

SEQ ID NO: 24 - protein, 15 aa, Artificial Sequence
  Peptide M_24
TNILLNVPLHGTILT

SEQ ID NO: 25 - protein, 15 aa, Artificial Sequence
  Peptide M_25
NVPLHGTILTRPLLE

SEQ ID NO: 26 - protein, 15 aa, Artificial Sequence
  Peptide M_26
GTILTRPLLESELVI

SEQ ID NO: 27 - protein, 15 aa, Artificial Sequence
  Peptide M_27
RPLLESELVIGAVIL

SEQ ID NO: 28 - protein, 15 aa, Artificial Sequence
  Peptide M_28
SELVIGAVILRGHLR

SEQ ID NO: 29 - protein, 15 aa, Artificial Sequence
  Peptide M_29
GAVILRGHLRIAGHH

SEQ ID NO: 30 - protein, 15 aa, Artificial Sequence
  Peptide M_30
RGHLRIAGHHLGRCD

SEQ ID NO: 31 - protein, 15 aa, Artificial Sequence
  Peptide M_30
IAGHHLGRCDIKDLP

SEQ ID NO: 32 - protein, 15 aa, Artificial Sequence
  Peptide M_32
LGRCDIKDLPKEITV

SEQ ID NO: 33 - protein, 15 aa, Artificial Sequence
  Peptide M_33
IKDLPKEITVATSRT

SEQ ID NO: 34 - protein, 15 aa, Artificial Sequence
  Peptide M_34
KEITVATSRTLSYYK

SEQ ID NO: 35 - protein, 15 aa, Artificial Sequence
  Peptide M_35
ATSRTLSYYKLGASQ

SEQ ID NO: 36 - protein, 15 aa, Artificial Sequence
  Peptide M_36
LSYYKLGASQRVAGD

SEQ ID NO: 37 - protein, 15 aa, Artificial Sequence
  Peptide M_37
LGASQRVAGDSGFAA

SEQ ID NO: 38 - protein, 15 aa, Artificial Sequence
  Peptide M_38
RVAGDSGFAAYSRYR

SEQ ID NO: 39 - protein, 15 aa, Artificial Sequence
  Peptide M_39
SGFAAYSRYRIGNYK

SEQ ID NO: 40 - protein, 15 aa, Artificial Sequence
  Peptide M_40
YSRYRIGNYKLNTDH

SEQ ID NO: 41 - protein, 15 aa, Artificial Sequence
  Peptide M_41
IGNYKLNTDHSSSSD

SEQ ID NO: 42 - protein, 15 aa, Artificial Sequence
  Peptide M_42
LNTDHSSSSDNIALL

SEQ ID NO: 43 - protein, 12 aa, Artificial Sequence
  Peptide M_43
SSSSDNIALLVQ

SEQ ID NO: 44 - protein, 15 aa, Artificial Sequence
  Peptide NP_1
MSDNGPQNQRNAPRI

SEQ ID NO: 45 - protein, 15 aa, Artificial Sequence
  Peptide NP_2
PQNQRNAPRITFGGP

SEQ ID NO: 46 - protein, 15 aa, Artificial Sequence
  Peptide NP_3
NAPRITFGGPSDSTG

SEQ ID NO: 47 - protein, 15 aa, Artificial Sequence
  Peptide NP_4
TFGGPSDSTGSNQNG

SEQ ID NO: 48 - protein, 15 aa, Artificial Sequence
  Peptide NP_5
SDSTGSNQNGERSGA

SEQ ID NO: 49 - protein, 15 aa, Artificial Sequence
  Peptide NP_6
SNQNGERSGARSKQR

SEQ ID NO: 50 - protein, 15 aa, Artificial Sequence
  Peptide NP_7
ERSGARSKQRRPQGL

SEQ ID NO: 51 - protein, 15 aa, Artificial Sequence
  Peptide NP_8
RSKQRRPQGLPNNTA

SEQ ID NO: 52 - protein, 15 aa, Artificial Sequence
  Peptide NP_9
RPQGLPNNTASWFTA

SEQ ID NO: 53 - protein, 15 aa, Artificial Sequence
  Peptide NP_10
PNNTASWFTALTQHG

SEQ ID NO: 54 - protein, 15 aa, Artificial Sequence
  Peptide NP_11
SWFTALTQHGKEDLK

SEQ ID NO: 55 - protein, 15 aa, Artificial Sequence
  Peptide NP_12
LTQHGKEDLKFPRGQ

SEQ ID NO: 56 - protein, 15 aa, Artificial Sequence
  Peptide NP_13
KEDLKFPRGQGVPIN

SEQ ID NO: 57 - protein, 15 aa, Artificial Sequence
  Peptide NP_14
FPRGQGVPINTNSSP

SEQ ID NO: 58 - protein, 15 aa, Artificial Sequence
  Peptide NP_15
GVPINTNSSPDDQIG

SEQ ID NO: 59 - protein, 15 aa, Artificial Sequence
  Peptide NP_16
TNSSPDDQIGYYRRA

SEQ ID NO: 60 - protein, 15 aa, Artificial Sequence
  Peptide NP_17
DDQIGYYRRATRRIR

SEQ ID NO: 61 - protein, 15 aa, Artificial Sequence
  Peptide NP_18
YYRRATRRIRGGDGK

SEQ ID NO: 62 - protein, 15 aa, Artificial Sequence
  Peptide NP_19
TRRIRGGDGKMKDLS

SEQ ID NO: 63 - protein, 15 aa, Artificial Sequence
  Peptide NP_20
GGDGKMKDLSPRWYF

SEQ ID NO: 64 - protein, 15 aa, Artificial Sequence
  Peptide NP_21
MKDLSPRWYFYYLGT

SEQ ID NO: 65 - protein, 15 aa, Artificial Sequence
  Peptide NP_22
PRWYFYYLGTGPEAG

SEQ ID NO: 66 - protein, 15 aa, Artificial Sequence
  Peptide NP_23
YYLGTGPEAGLPYGA

SEQ ID NO: 67 - protein, 15 aa, Artificial Sequence
  Peptide NP_24
GPEAGLPYGANKDGI

SEQ ID NO: 68 - protein, 15 aa, Artificial Sequence
  Peptide NP_25
LPYGANKDGIIWVAT

SEQ ID NO: 69 - protein, 15 aa, Artificial Sequence
  Peptide NP_26
NKDGIIWVATEGALN

SEQ ID NO: 70 - protein, 15 aa, Artificial Sequence
  Peptide NP_27
IWVATEGALNTPKDH

SEQ ID NO: 71 - protein, 15 aa, Artificial Sequence
  Peptide NP_28
EGALNTPKDHIGTRN

SEQ ID NO: 72 - protein, 15 aa, Artificial Sequence
  Peptide NP_29
TPKDHIGTRNPANNA

SEQ ID NO: 73 - protein, 15 aa, Artificial Sequence
  Peptide NP_30
IGTRNPANNAAIVLQ

SEQ ID NO: 74 - protein, 15 aa, Artificial Sequence
  Peptide NP_31
PANNAAIVLQLPQGT

SEQ ID NO: 75 - protein, 15 aa, Artificial Sequence
  Peptide NP_32
AIVLQLPQGTTLPKG

SEQ ID NO: 76 - protein, 15 aa, Artificial Sequence
  Peptide NP_33
LPQGTTLPKGFYAEG

SEQ ID NO: 77 - protein, 15 aa, Artificial Sequence
  Peptide NP_34
TLPKGFYAEGSRGGS

SEQ ID NO: 78 - protein, 15 aa, Artificial Sequence
  Peptide NP_35
FYAEGSRGGSQASSR

SEQ ID NO: 79 - protein, 15 aa, Artificial Sequence
  Peptide NP_36
SRGGSQASSRSSSRS

SEQ ID NO: 80 - protein, 15 aa, Artificial Sequence
  Peptide NP_37
QASSRSSSRSRNSSR

SEQ ID NO: 81 - protein, 15 aa, Artificial Sequence
  Peptide NP_38
SSSRSRNSSRNSTPG

SEQ ID NO: 82 - protein, 15 aa, Artificial Sequence
  Peptide NP_39
RNSSRNSTPGSSRGT

SEQ ID NO: 83 - protein, 15 aa, Artificial Sequence
  Peptide NP_40
NSTPGSSRGTSPARM

SEQ ID NO: 84 - protein, 15 aa, Artificial Sequence
  Peptide NP_41
SSRGTSPARMAGNGG

SEQ ID NO: 85 - protein, 15 aa, Artificial Sequence
  Peptide NP_42
SPARMAGNGGDAALA

SEQ ID NO: 86 - protein, 15 aa, Artificial Sequence
  Peptide NP_43
AGNGGDAALALLLLD

SEQ ID NO: 87 - protein, 15 aa, Artificial Sequence
  Peptide NP_44
DAALALLLLDRLNQL

SEQ ID NO: 88 - protein, 15 aa, Artificial Sequence
  Peptide NP_45
LLLLDRLNQLESKMS

SEQ ID NO: 89 - protein, 15 aa, Artificial Sequence
  Peptide NP_46
RLNQLESKMSGKGQQ

SEQ ID NO: 90 - protein, 15 aa, Artificial Sequence
  Peptide NP_47
ESKMSGKGQQQQGQT

SEQ ID NO: 91 - protein, 15 aa, Artificial Sequence
  Peptide NP_48
GKGQQQQGQTVTKKS

SEQ ID NO: 92 - protein, 15 aa, Artificial Sequence
  Peptide NP_49
QQGQTVTKKSAAEAS

SEQ ID NO: 93 - protein, 15 aa, Artificial Sequence
  Peptide NP_50
VTKKSAAEASKKPRQ

SEQ ID NO: 94 - protein, 15 aa, Artificial Sequence
  Peptide NP_51
AAEASKKPRQKRTAT

SEQ ID NO: 95 - protein, 15 aa, Artificial Sequence
  Peptide NP_52
KKPRQKRTATKAYNV

SEQ ID NO: 96 - protein, 15 aa, Artificial Sequence
  Peptide NP_53
KRTATKAYNVTQAFG

SEQ ID NO: 97 - protein, 15 aa, Artificial Sequence
  Peptide NP_54
KAYNVTQAFGRRGPE

SEQ ID NO: 98 - protein, 15 aa, Artificial Sequence
  Peptide NP_55
TQAFGRRGPEQTQGN

SEQ ID NO: 99 - protein, 15 aa, Artificial Sequence
  Peptide NP_56
RRGPEQTQGNFGDQE

SEQ ID NO: 100 - protein, 15 aa, Artificial Sequence
  Peptide NP_57
QTQGNFGDQELIRQG

SEQ ID NO: 101 - protein, 15 aa, Artificial Sequence
  Peptide NP_58
FGDQELIRQGTDYKH

SEQ ID NO: 102 - protein, 15 aa, Artificial Sequence
  Peptide NP_59
LIRQGTDYKHWPQIA

SEQ ID NO: 103 - protein, 15 aa, Artificial Sequence
  Peptide NP_60
TDYKHWPQIAQFAPS

SEQ ID NO: 104 - protein, 15 aa, Artificial Sequence
  Peptide NP_61
WPQIAQFAPSASAFF

SEQ ID NO: 105 - protein, 15 aa, Artificial Sequence
  Peptide NP_62
QFAPSASAFFGMSRI

SEQ ID NO: 106 - protein, 15 aa, Artificial Sequence
  Peptide NP_63
ASAFFGMSRIGMEVT

SEQ ID NO: 107 - protein, 15 aa, Artificial Sequence
  Peptide NP_64
GMSRIGMEVTPSGTW

SEQ ID NO: 108 - protein, 15 aa, Artificial Sequence
  Peptide NP_65
GMEVTPSGTWLTYTG

SEQ ID NO: 109 - protein, 15 aa, Artificial Sequence
  Peptide NP_66
PSGTWLTYTGAIKLD

SEQ ID NO: 110 - protein, 15 aa, Artificial Sequence
  Peptide NP_67
LTYTGAIKLDDKDPN

SEQ ID NO: 111 - protein, 15 aa, Artificial Sequence
  Peptide NP_68
AIKLDDKDPNFKDQV

SEQ ID NO: 112 - protein, 15 aa, Artificial Sequence
  Peptide NP_69
DKDPNFKDQVILLNK

SEQ ID NO: 113 - protein, 15 aa, Artificial Sequence
  Peptide NP_70
FKDQVILLNKHIDAY

SEQ ID NO: 114 - protein, 15 aa, Artificial Sequence
  Peptide NP_71
ILLNKHIDAYKTFPP

SEQ ID NO: 115 - protein, 15 aa, Artificial Sequence
  Peptide NP_72
HIDAYKTFPPTEPKK

SEQ ID NO: 116 - protein, 15 aa, Artificial Sequence
  Peptide NP_73
KTFPPTEPKKDKKKK

SEQ ID NO: 117 - protein, 15 aa, Artificial Sequence
  Peptide NP_74
TEPKKDKKKKADETQ

SEQ ID NO: 118 - protein, 15 aa, Artificial Sequence
  Peptide NP_75
DKKKKADETQALPQR

SEQ ID NO: 119 - protein, 15 aa, Artificial Sequence
  Peptide NP_76
ADETQALPQRQKKQQ

SEQ ID NO: 120 - protein, 15 aa, Artificial Sequence
  Peptide NP_77
ALPQRQKKQQTVTLL

SEQ ID NO: 121 - protein, 15 aa, Artificial Sequence
  Peptide NP_78
QKKQQTVTLLPAADL

SEQ ID NO: 122 - protein, 15 aa, Artificial Sequence
  Peptide NP_79
TVTLLPAADLDDFSK

SEQ ID NO: 123 - protein, 15 aa, Artificial Sequence
  Peptide NP_80
PAADLDDFSKQLQQS

SEQ ID NO: 124 - protein, 15 aa, Artificial Sequence
  Peptide NP_81
DDFSKQLQQSMSSAD

SEQ ID NO: 125 - protein, 14 aa, Artificial Sequence
  Peptide NP_82
QLQQSMSSADSTQA

SEQ ID NO: 126 - protein, 15 aa, Artificial Sequence
  Peptide SP_21
IRGWIFGTTLDSKTQ

SEQ ID NO: 127 - protein, 15 aa, Artificial Sequence
  Peptide SP_22
FGTTLDSKTQSLLIV

SEQ ID NO: 128 - protein, 15 aa, Artificial Sequence
  Peptide SP_34
CTFEYVSQPFLMDLE

SEQ ID NO: 129 - protein, 15 aa, Artificial Sequence
  Peptide SP_35
VSQPFLMDLEGKQGN

SEQ ID NO: 130 - protein, 15 aa, Artificial Sequence
  Peptide SP_48
TRFQTLLALHRSYLT

SEQ ID NO: 131 - protein, 15 aa, Artificial Sequence
  Peptide SP_49
LLALHRSYLTPGDSS

SEQ ID NO: 132 - protein, 15 aa, Artificial Sequence
  Peptide SP_50
RSYLTPGDSSSGWTA

SEQ ID NO: 133 - protein, 15 aa, Artificial Sequence
  Peptide SP_59
CALDPLSETKCTLKS

SEQ ID NO: 134 - protein, 15 aa, Artificial Sequence
  Peptide SP_60
LSETKCTLKSFTVEK

SEQ ID NO: 135 - protein, 15 aa, Artificial Sequence
  Peptide SP_61
CTLKSFTVEKGIYQT

SEQ ID NO: 136 - protein, 15 aa, Artificial Sequence
  Peptide SP_62
FTVEKGIYQTSNFRV

SEQ ID NO: 137 - protein, 15 aa, Artificial Sequence
  Peptide SP_63
GIYQTSNFRVQPTES

SEQ ID NO: 138 - protein, 15 aa, Artificial Sequence
  Peptide SP_75
SASFSTFKCYGVSPT

SEQ ID NO: 139 - protein, 15 aa, Artificial Sequence
  Peptide SP_76
TFKCYGVSPTKLNDL

SEQ ID NO: 140 - protein, 15 aa, Artificial Sequence
  Peptide SP_85
YNYKLPDDFTGCVIA

SEQ ID NO: 141 - protein, 15 aa, Artificial Sequence
  Peptide SP_88
WNSNNLDSKVGGNYN

SEQ ID NO: 142 - protein, 15 aa, Artificial Sequence
  Peptide SP_89
LDSKVGGNYNYLYRL

SEQ ID NO: 143 - protein, 15 aa, Artificial Sequence
  Peptide SP_90
GGNYNYLYRLFRKSN

SEQ ID NO: 144 - protein, 15 aa, Artificial Sequence
  Peptide SP_91
YLYRLFRKSNLKPFE

SEQ ID NO: 145 - protein, 15 aa, Artificial Sequence
  Peptide SP_92
FRKSNLKPFERDIST

SEQ ID NO: 146 - protein, 15 aa, Artificial Sequence
  Peptide SP_93
LKPFERDISTEIYQA

SEQ ID NO: 147 - protein, 15 aa, Artificial Sequence
  Peptide SP_106
GPKKSTNLVKNKCVN

SEQ ID NO: 148 - protein, 15 aa, Artificial Sequence
  Peptide SP_107
TNLVKNKCVNFNFNG

SEQ ID NO: 149 - protein, 15 aa, Artificial Sequence
  Peptide SP_109
FNFNGLTGTGVLTES

SEQ ID NO: 150 - protein, 15 aa, Artificial Sequence
  Peptide SP_110
LTGTGVLTESNKKFL

SEQ ID NO: 151 - protein, 15 aa, Artificial Sequence
  Peptide SP_130
RAGCLIGAEHVNNSY

SEQ ID NO: 152 - protein, 15 aa, Artificial Sequence
  Peptide SP_131
IGAEHVNNSYECDIP

SEQ ID NO: 153 - protein, 15 aa, Artificial Sequence
  Peptide SP_138
SVASQSIIAYTMSLG

SEQ ID NO: 154 - protein, 15 aa, Artificial Sequence
  Peptide SP_139
SIIAYTMSLGAENSV

SEQ ID NO: 155 - protein, 15 aa, Artificial Sequence
  Peptide SP_140
TMSLGAENSVAYSNN

SEQ ID NO: 156 - protein, 15 aa, Artificial Sequence
  Peptide SP_150
STECSNLLLQYGSFC

SEQ ID NO: 157 - protein, 15 aa, Artificial Sequence
  Peptide SP_151
NLLLQYGSFCTQLNR

SEQ ID NO: 158 - protein, 15 aa, Artificial Sequence
  Peptide SP_156
KNTQEVFAQVKQIYK

SEQ ID NO: 159 - protein, 15 aa, Artificial Sequence
  Peptide SP_157
VFAQVKQIYKTPPIK

SEQ ID NO: 160 - protein, 15 aa, Artificial Sequence
  Peptide SP_158
KQIYKTPPIKDFGGF

SEQ ID NO: 161 - protein, 15 aa, Artificial Sequence
  Peptide SP_159
TPPIKDFGGFNFSQI

SEQ ID NO: 162 - protein, 15 aa, Artificial Sequence
  Peptide SP_161
NFSQILPDPSKPSKR

SEQ ID NO: 163 - protein, 15 aa, Artificial Sequence
  Peptide SP_167
AGFIKQYGDCLGDIA

SEQ ID NO: 164 - protein, 15 aa, Artificial Sequence
  Peptide SP_168
QYGDCLGDIAARDLI

SEQ ID NO: 165 - protein, 15 aa, Artificial Sequence
  Peptide SP_179
GAALQIPFAMQMAYR

SEQ ID NO: 166 - protein, 15 aa, Artificial Sequence
  Peptide SP_181
QMAYRFNGIGVTQNV

SEQ ID NO: 167 - protein, 15 aa, Artificial Sequence
  Peptide SP_182
FNGIGVTQNVLYENQ

SEQ ID NO: 168 - protein, 15 aa, Artificial Sequence
  Peptide SP_188
DSLSSTASALGKLQD

SEQ ID NO: 169 - protein, 15 aa, Artificial Sequence
  Peptide SP_189
TASALGKLQDVVNQN

SEQ ID NO: 170 - protein, 15 aa, Artificial Sequence
  Peptide SP_192
AQALNTLVKQLSSNF

SEQ ID NO: 171 - protein, 15 aa, Artificial Sequence
  Peptide SP_196
VLNDILSRLDKVEAE

SEQ ID NO: 172 - protein, 15 aa, Artificial Sequence
  Peptide SP_200
LITGRLQSLQTYVTQ

SEQ ID NO: 173 - protein, 15 aa, Artificial Sequence
  Peptide SP_203
QLIRAAEIRASANLA

SEQ ID NO: 174 - protein, 15 aa, Artificial Sequence
  Peptide SP_204
AEIRASANLAATKMS

SEQ ID NO: 175 - protein, 15 aa, Artificial Sequence
  Peptide SP_212
APHGVVFLHVTYVPA

SEQ ID NO: 176 - protein, 15 aa, Artificial Sequence
  Peptide SP_221
HWFVTQRNFYEPQII

SEQ ID NO: 177 - protein, 15 aa, Artificial Sequence
  Peptide SP_237
KEIDRLNEVAKNLNE

SEQ ID NO: 178 - protein, 15 aa, Artificial Sequence
  Peptide SP_238
LNEVAKNLNESLIDL

SEQ ID NO: 179 - protein, 15 aa, Artificial Sequence
  Peptide SP_239
KNLNESLIDLQELGK

SEQ ID NO: 180 - protein, 15 aa, Artificial Sequence
  Peptide SP_244
IWLGFIAGLIAIVMV

SEQ ID NO: 181 - protein, 15 aa, Artificial Sequence
  Peptide SP_70
RFASVYAWNRKRISN

SEQ ID NO: 182 - protein, 15 aa, Artificial Sequence
  Peptide SP_71
YAWNRKRISNCVADY

SEQ ID NO: 183 - protein, 15 aa, Artificial Sequence
  Sequence 21A SP_002
VLLPLVSSQCVNLRT

SEQ ID NO: 184 - protein, 15 aa, Artificial Sequence
  Sequence 21A SP_003
VSSQCVNLRTRTQLP

SEQ ID NO: 185 - protein, 15 aa, Artificial Sequence
  Sequence 21A SP_004
VNLRTRTQLPPAYTN

SEQ ID NO: 186 - protein, 15 aa, Artificial Sequence
  Sequence 21A SP_027
CEFQFCNDPFLDVYY

SEQ ID NO: 187 - protein, 15 aa, Artificial Sequence
  Sequence 21A SP_028
CNDPFLDVYYHKNNK

SEQ ID NO: 188 - protein, 15 aa, Artificial Sequence
  Sequence 21A SP_029
LDVYYHKNNKSWMES

SEQ ID NO: 189 - protein, 15 aa, Artificial Sequence
  Sequence 21A SP_030
HKNNKSWMESGVYSS

SEQ ID NO: 190 - protein, 15 aa, Artificial Sequence
  Sequence 21A SP_031
SWMESGVYSSANNCT

SEQ ID NO: 191 - protein, 15 aa, Artificial Sequence
  Sequence 21A SP_032
GVYSSANNCTFEYVS

SEQ ID NO: 192 - protein, 15 aa, Artificial Sequence
  Sequence 21A SP_089
LDSKVGGNYNYRYRL

SEQ ID NO: 193 - protein, 15 aa, Artificial Sequence
  Sequence 21A SP_090
GGNYNYRYRLFRKSN

SEQ ID NO: 194 - protein, 15 aa, Artificial Sequence
  Sequence 21A SP_091
YRYRLFRKSNLKPFE

SEQ ID NO: 195 - protein, 15 aa, Artificial Sequence
  Sequence 21A SP_094
RDISTEIYQAGSKPC

SEQ ID NO: 196 - protein, 15 aa, Artificial Sequence
  Sequence 21A SP_095
EIYQAGSKPCNGVEG

SEQ ID NO: 197 - protein, 15 aa, Artificial Sequence
  Sequence 21A SP_096
GSKPCNGVEGFNCYF

SEQ ID NO: 198 - protein, 15 aa, Artificial Sequence
  Sequence 21A SP_121
GTNTSNQVAVLYQGV

SEQ ID NO: 199 - protein, 15 aa, Artificial Sequence
  Sequence 21A SP_122
NQVAVLYQGVNCTEV

SEQ ID NO: 200 - protein, 15 aa, Artificial Sequence
  Sequence 21A SP_123
LYQGVNCTEVPVAIH

SEQ ID NO: 201 - protein, 15 aa, Artificial Sequence
  Sequence 21A SP_135
CASYQTQTNSRRRAR

SEQ ID NO: 202 - protein, 15 aa, Artificial Sequence
  Sequence 21A SP_136
TQTNSRRRARSVASQ

SEQ ID NO: 203 - protein, 15 aa, Artificial Sequence
  Sequence 21A SP_137
RRRARSVASQSIIAY

SEQ ID NO: 204 - protein, 15 aa, Artificial Sequence
  Sequence 21A SP_188
DSLSSTASALGKLQN

SEQ ID NO: 205 - protein, 15 aa, Artificial Sequence
  Sequence 21A SP_189
TASALGKLQNVVNQN

SEQ ID NO: 206 - protein, 15 aa, Artificial Sequence
  Sequence 21A SP_190
GKLQNVVNQNAQALN

SEQ ID NO: 207 - protein, 15 aa, Artificial Sequence
  SEQUENCE_20I SP_012
LPFFSNVTWFHAISG

SEQ ID NO: 208 - protein, 15 aa, Artificial Sequence
  SEQUENCE_20I SP_013
NVTWFHAISGTNGTK

SEQ ID NO: 209 - protein, 15 aa, Artificial Sequence
  SEQUENCE_20I SP_014
HAISGTNGTKRFDNP

SEQ ID NO: 210 - protein, 15 aa, Artificial Sequence
  SEQUENCE_20I SP_027
CEFQFCNDPFLGVYH

SEQ ID NO: 211 - protein, 15 aa, Artificial Sequence
  SEQUENCE_20I SP_028
CNDPFLGVYHKNNKS

SEQ ID NO: 212 - protein, 15 aa, Artificial Sequence
  SEQUENCE_20I SP_029
LGVYHKNNKSWMESE

SEQ ID NO: 213 - protein, 15 aa, Artificial Sequence
  SEQUENCE_20I SP_095
EIYQAGSTPCNGVKG

SEQ ID NO: 214 - protein, 15 aa, Artificial Sequence
  SEQUENCE_20I SP_096
GSTPCNGVKGFNCYF

SEQ ID NO: 215 - protein, 15 aa, Artificial Sequence
  SEQUENCE_20I SP_097
NGVKGFNCYFPLQPY

SEQ ID NO: 216 - protein, 15 aa, Artificial Sequence
  SEQUENCE_20I SP_098
FNCYFPLQPYGFQPT

SEQ ID NO: 217 - protein, 15 aa, Artificial Sequence
  SEQUENCE_20I SP_099
PLQPYGFQPTYGVGY

SEQ ID NO: 218 - protein, 15 aa, Artificial Sequence
  SEQUENCE_20I SP_100
GFQPTYGVGYQPYRV

SEQ ID NO: 219 - protein, 15 aa, Artificial Sequence
  SEQUENCE_20I SP_101
YGVGYQPYRVVVLSF

SEQ ID NO: 220 - protein, 15 aa, Artificial Sequence
  SEQUENCE_20I SP_112
NKKFLPFQQFGRDID

SEQ ID NO: 221 - protein, 15 aa, Artificial Sequence
  SEQUENCE_20I SP_113
PFQQFGRDIDDTTDA

SEQ ID NO: 222 - protein, 15 aa, Artificial Sequence
  SEQUENCE_20I SP_114
GRDIDDTTDAVRDPQ

SEQ ID NO: 223 - protein, 15 aa, Artificial Sequence
  SEQUENCE_20I SP_121
GTNTSNQVAVLYQGV

SEQ ID NO: 224 - protein, 15 aa, Artificial Sequence
  SEQUENCE_20I SP_122
NQVAVLYQGVNCTEV

SEQ ID NO: 225 - protein, 15 aa, Artificial Sequence
  SEQUENCE_20I SP_123
LYQGVNCTEVPVAIH

SEQ ID NO: 226 - protein, 15 aa, Artificial Sequence
  SEQUENCE_20I SP_135
CASYQTQTNSHRRAR

SEQ ID NO: 227 - protein, 15 aa, Artificial Sequence
  SEQUENCE_20I SP_136
TQTNSHRRARSVASQ

SEQ ID NO: 228 - protein, 15 aa, Artificial Sequence
  SEQUENCE_20I SP_137
HRRARSVASQSIIAY

SEQ ID NO: 229 - protein, 15 aa, Artificial Sequence
  SEQUENCE_20I SP_142
AYSNNSIAIPINFTI

SEQ ID NO: 230 - protein, 15 aa, Artificial Sequence
  SEQUENCE_20I SP_143
SIAIPINFTISVTTE

SEQ ID NO: 231 - protein, 15 aa, Artificial Sequence
  SEQUENCE_20I SP_144
INFTISVTTEILPVS

SEQ ID NO: 232 - protein, 15 aa, Artificial Sequence
  SEQUENCE_20I SP_195
GAISSVLNDILARLD

SEQ ID NO: 233 - protein, 15 aa, Artificial Sequence
  SEQUENCE_20I SP_196
VLNDILARLDKVEAE

SEQ ID NO: 234 - protein, 15 aa, Artificial Sequence
  SEQUENCE_20I SP_197
LARLDKVEAEVQIDR

SEQ ID NO: 235 - protein, 15 aa, Artificial Sequence
  SEQUENCE_20I SP_222
QRNFYEPQIITTHNT

SEQ ID NO: 236 - protein, 15 aa, Artificial Sequence
  SEQUENCE_20I SP_223
EPQIITTHNTFVSGN

SEQ ID NO: 237 - protein, 15 aa, Artificial Sequence
  SEQUENCE_20I SP_224
TTHNTFVSGNCDVVI

SEQ ID NO: 238 - protein, 15 aa, Artificial Sequence
  SEQUENCE_20I SP_237
KEIDRLNEVANNLNE

SEQ ID NO: 239 - protein, 15 aa, Artificial Sequence
  SEQUENCE_20I SP_238
LNEVANNLNESLIDL

SEQ ID NO: 240 - protein, 15 aa, Artificial Sequence
  SEQUENCE_20I SP_239
NNLNESLIDLQELGK

SEQ ID NO: 241 - protein, 15 aa, Artificial Sequence
  SEQUENCE 20H SP_002
VLLPLVSSQCVNFTT

SEQ ID NO: 242 - protein, 15 aa, Artificial Sequence
  SEQUENCE 20H SP_003
VSSQCVNFTTRTQLP

SEQ ID NO: 243 - protein, 15 aa, Artificial Sequence
  SEQUENCE 20H SP_004
VNFTTRTQLPPAYTN

SEQ ID NO: 244 - protein, 15 aa, Artificial Sequence
  SEQUENCE 20H SP_014
HAIHVSGTNGTKRFA

SEQ ID NO: 245 - protein, 15 aa, Artificial Sequence
  SEQUENCE 20H SP_015
SGTNGTKRFANPVLP

SEQ ID NO: 246 - protein, 15 aa, Artificial Sequence
  SEQUENCE 20H SP_016
TKRFANPVLPFNDGV

SEQ ID NO: 247 - protein, 15 aa, Artificial Sequence
  SEQUENCE 20H SP_041
FKIYSKHTPINLVRG

SEQ ID NO: 248 - protein, 15 aa, Artificial Sequence
  SEQUENCE 20H SP_042
KHTPINLVRGLPQGF

SEQ ID NO: 249 - protein, 15 aa, Artificial Sequence
  SEQUENCE 20H SP_043
NLVRGLPQGFSALEP

SEQ ID NO: 250 - protein, 15 aa, Artificial Sequence
  SEQUENCE 20H SP_047
IGINITRFQTLHRSY

SEQ ID NO: 251 - protein, 15 aa, Artificial Sequence
  SEQUENCE 20H SP_048
TRFQTLHRSYLTPGD

SEQ ID NO: 252 - protein, 15 aa, Artificial Sequence
  SEQUENCE 20H SP_048
LHRSYLTPGDSSSGW

SEQ ID NO: 253 - protein, 15 aa, Artificial Sequence
  SEQUENCE 20H SP_082
EVRQIAPGQTGNIAD

SEQ ID NO: 254 - protein, 15 aa, Artificial Sequence
  SEQUENCE 20H SP_083
APGQTGNIADYNYKL

SEQ ID NO: 255 - protein, 15 aa, Artificial Sequence
  SEQUENCE 20H SP_084
GNIADYNYKLPDDFT

SEQ ID NO: 256 - protein, 15 aa, Artificial Sequence
  SEQUENCE 20H SP_095
EIYQAGSTPCNGVKG

SEQ ID NO: 257 - protein, 15 aa, Artificial Sequence
  SEQUENCE 20H SP_096
GSTPCNGVKGFNCYF

SEQ ID NO: 258 - protein, 15 aa, Artificial Sequence
  SEQUENCE 20H SP_097
NGVKGFNCYFPLQSY

SEQ ID NO: 259 - protein, 15 aa, Artificial Sequence
  SEQUENCE 20H SP_099
PLQSYGFQPTYGVGY

SEQ ID NO: 260 - protein, 15 aa, Artificial Sequence
  SEQUENCE 20H SP_100
GFQPTYGVGYQPYRV

SEQ ID NO: 261 - protein, 15 aa, Artificial Sequence
  SEQUENCE 20H SP_101
YGVGYQPYRVVVLSF

SEQ ID NO: 262 - protein, 15 aa, Artificial Sequence
  SEQUENCE 20H SP_121
GTNTSNQVAVLYQGV

SEQ ID NO: 263 - protein, 15 aa, Artificial Sequence
  SEQUENCE 20H SP_122
NQVAVLYQGVNCTEV

SEQ ID NO: 264 - protein, 15 aa, Artificial Sequence
  SEQUENCE 20H SP_123
LYQGVNCTEVPVAIH

SEQ ID NO: 265 - protein, 15 aa, Artificial Sequence
  SEQUENCE 20H SP_139
SIIAYTMSLGVENSV

SEQ ID NO: 266 - protein, 15 aa, Artificial Sequence
  SEQUENCE 20H SP_140
TMSLGVENSVAYSNN

SEQ ID NO: 267 - protein, 15 aa, Artificial Sequence
  SEQUENCE 20H SP_141
VENSVAYSNNSIAIP

SEQ ID NO: 268 - protein, 15 aa, Artificial Sequence
  SEQUENCE_20J SP_002
VLLPLVSSQCVNFTN

SEQ ID NO: 269 - protein, 15 aa, Artificial Sequence
  SEQUENCE_20J SP_003
VSSQCVNFTNRTQLP

SEQ ID NO: 270 - protein, 15 aa, Artificial Sequence
  SEQUENCE_20J SP_004
VNFTNRTQLPSAYTN

SEQ ID NO: 271 - protein, 15 aa, Artificial Sequence
  SEQUENCE_20J SP_005
RTQLPSAYTNSFTRG

SEQ ID NO: 272 - protein, 15 aa, Artificial Sequence
  SEQUENCE_20J SP_006
SAYTNSFTRGVYYPD

SEQ ID NO: 273 - protein, 15 aa, Artificial Sequence
  SEQUENCE_20J SP_026
VVIKVCEFQFCNYPF

SEQ ID NO: 274 - protein, 15 aa, Artificial Sequence
  SEQUENCE_20J SP_027
CEFQFCNYPFLGVYY

SEQ ID NO: 275 - protein, 15 aa, Artificial Sequence
  SEQUENCE_20J SP_028
CNYPFLGVYYHKNNK

SEQ ID NO: 276 - protein, 15 aa, Artificial Sequence
  SEQUENCE_20J SP_036
LMDLEGKQGNFKNLS

SEQ ID NO: 277 - protein, 15 aa, Artificial Sequence
  SEQUENCE_20J SP_037
GKQGNFKNLSEFVFK

SEQ ID NO: 278 - protein, 15 aa, Artificial Sequence
  SEQUENCE_20J SP_038
FKNLSEFVFKNIDGY

SEQ ID NO: 279 - protein, 15 aa, Artificial Sequence
  SEQUENCE_20J SP_082
EVRQIAPGQTGTIAD

SEQ ID NO: 280 - protein, 15 aa, Artificial Sequence
  SEQUENCE_20J SP_083
APGQTGTIADYNYKL

SEQ ID NO: 281 - protein, 15 aa, Artificial Sequence
  SEQUENCE_20J SP_084
GTIADYNYKLPDDFT

SEQ ID NO: 282 - protein, 15 aa, Artificial Sequence
  SEQUENCE_20J SP_095
EIYQAGSTPCNGVKG

SEQ ID NO: 283 - protein, 15 aa, Artificial Sequence
  SEQUENCE_20J SP_096
GSTPCNGVKGFNCYF

SEQ ID NO: 284 - protein, 15 aa, Artificial Sequence
  SEQUENCE_20J SP_097
NGVKGFNCYFPLQSY

SEQ ID NO: 285 - protein, 15 aa, Artificial Sequence
  SEQUENCE_20J SP_099
PLQSYGFQPTYGVGY

SEQ ID NO: 286 - protein, 15 aa, Artificial Sequence
  SEQUENCE_20J SP_100
GFQPTYGVGYQPYRV

SEQ ID NO: 287 - protein, 15 aa, Artificial Sequence
  SEQUENCE_20J SP_101
YGVGYQPYRVVVLSF

SEQ ID NO: 288 - protein, 15 aa, Artificial Sequence
  SEQUENCE_20J SP_121
GTNTSNQVAVLYQGV

SEQ ID NO: 289 - protein, 15 aa, Artificial Sequence
  SEQUENCE_20J SP_122
NQVAVLYQGVNCTEV

SEQ ID NO: 290 - protein, 15 aa, Artificial Sequence
  SEQUENCE_20J SP_123
LYQGVNCTEVPVAIH

SEQ ID NO: 291 - protein, 15 aa, Artificial Sequence
  SEQUENCE_20J SP_129
NVFQTRAGCLIGAEY

SEQ ID NO: 292 - protein, 15 aa, Artificial Sequence
  SEQUENCE_20J SP_130
RAGCLIGAEYVNNSY

SEQ ID NO: 293 - protein, 15 aa, Artificial Sequence
  SEQUENCE_20J SP_131
IGAEYVNNSYECDIP

SEQ ID NO: 294 - protein, 15 aa, Artificial Sequence
  SEQUENCE_20J SP_204
AEIRASANLAAIKMS

SEQ ID NO: 295 - protein, 15 aa, Artificial Sequence
  SEQUENCE_20J SP_205
SANLAAIKMSECVLG

SEQ ID NO: 296 - protein, 15 aa, Artificial Sequence
  SEQUENCE_20J SP_206
AIKMSECVLGQSKRV

SEQ ID NO: 297 - protein, 15 aa, Artificial Sequence
  SEQUENCE_20J SP_234
LGDISGINASFVNIQ

SEQ ID NO: 298 - protein, 15 aa, Artificial Sequence
  SEQUENCE_20J SP_235
GINASFVNIQKEIDR

SEQ ID NO: 299 - protein, 15 aa, Artificial Sequence
  SEQUENCE_20J SP_236
FVNIQKEIDRLNEVA

SEQ ID NO: 300 - protein, 15 aa, Artificial Sequence
  SEQUENCE_21B SP_017
NPVLPFNDGVYFASI

SEQ ID NO: 301 - protein, 15 aa, Artificial Sequence
  SEQUENCE_21B SP_018
FNDGVYFASIEKSNI

SEQ ID NO: 302 - protein, 15 aa, Artificial Sequence
  SEQUENCE_21B SP_019
YFASIEKSNIIRGWI

SEQ ID NO: 303 - protein, 15 aa, Artificial Sequence
  SEQUENCE_21B SP_027
CEFQFCNDPFLDVYY

SEQ ID NO: 304 - protein, 15 aa, Artificial Sequence
  SEQUENCE_21B SP_028
CNDPFLDVYYHKNNK

SEQ ID NO: 305 - protein, 15 aa, Artificial Sequence
  SEQUENCE_21B SP_029
LDVYYHKNNKSWMKS

SEQ ID NO: 306 - protein, 15 aa, Artificial Sequence
  SEQUENCE_21B SP_030
HKNNKSWMKSEFRVY

SEQ ID NO: 307 - protein, 15 aa, Artificial Sequence
  SEQUENCE_21B SP_031
SWMKSEFRVYSSANN

SEQ ID NO: 308 - protein, 15 aa, Artificial Sequence
  SEQUENCE_21B SP_089
LDSKVGGNYNYRYRL

SEQ ID NO: 309 - protein, 15 aa, Artificial Sequence
  SEQUENCE_21B SP_090
GGNYNYRYRLFRKSN

SEQ ID NO: 310 - protein, 15 aa, Artificial Sequence
  SEQUENCE_21B SP_091
YRYRLFRKSNLKPFE

SEQ ID NO: 311 - protein, 15 aa, Artificial Sequence
  SEQUENCE_21B SP_095
EIYQAGSTPCNGVQG

SEQ ID NO: 312 - protein, 15 aa, Artificial Sequence
  SEQUENCE_21B SP_096
GSTPCNGVQGFNCYF

SEQ ID NO: 313 - protein, 15 aa, Artificial Sequence
  SEQUENCE_21B SP_097
NGVQGFNCYFPLQSY

SEQ ID NO: 314 - protein, 15 aa, Artificial Sequence
  SEQUENCE_21B SP_121
GTNTSNQVAVLYQGV

SEQ ID NO: 315 - protein, 15 aa, Artificial Sequence
  SEQUENCE_21B SP_122
NQVAVLYQGVNCTEV

SEQ ID NO: 316 - protein, 15 aa, Artificial Sequence
  SEQUENCE_21B SP_123
LYQGVNCTEVPVAIH

SEQ ID NO: 317 - protein, 15 aa, Artificial Sequence
  SEQUENCE_21B SP_135
CASYQTQTNSRRRAR

SEQ ID NO: 318 - protein, 15 aa, Artificial Sequence
  SEQUENCE_21B SP_136
TQTNSRRRARSVASQ

SEQ ID NO: 319 - protein, 15 aa, Artificial Sequence
  SEQUENCE_21B SP_1237
RRRARSVASQSIIAY

SEQ ID NO: 320 - protein, 15 aa, Artificial Sequence
  SEQUENCE_21B SP_213
VFLHVTYVPAHEKNF

SEQ ID NO: 321 - protein, 15 aa, Artificial Sequence
  SEQUENCE_21B SP_214
TYVPAHEKNFTTAPA

SEQ ID NO: 322 - protein, 15 aa, Artificial Sequence
  SEQUENCE_21B SP_215
HEKNFTTAPAICHDG

SEQ ID NO: 323 - protein, 15 aa, Artificial Sequence
  SEQUENCE_20B/S.484K SP_095
EIYQAGSTPCNGVKG

SEQ ID NO: 324 - protein, 15 aa, Artificial Sequence
  SEQUENCE_20B/S.484K SP_096
GSTPCNGVKGFNCYF

SEQ ID NO: 325 - protein, 15 aa, Artificial Sequence
  SEQUENCE_20B/S.484K SP_097
NGVKGFNCYFPLQSY

SEQ ID NO: 326 - protein, 15 aa, Artificial Sequence
  SEQUENCE_20B/S.484K SP_121
GTNTSNQVAVLYQGV

SEQ ID NO: 327 - protein, 15 aa, Artificial Sequence
  SEQUENCE_20B/S.484K SP_122
NQVAVLYQGVNCTEV

SEQ ID NO: 328 - protein, 15 aa, Artificial Sequence
  SEQUENCE_20B/S.484K SP_123
LYQGVNCTEVPVAIH

SEQ ID NO: 329 - protein, 15 aa, Artificial Sequence
  SEQUENCE_20B/S.484K SP_234
LGDISGINASFVNIQ

SEQ ID NO: 330 - protein, 15 aa, Artificial Sequence
  SEQUENCE_20B/S.484K SP_235
GINASFVNIQKEIDR

SEQ ID NO: 331 - protein, 15 aa, Artificial Sequence
  SEQUENCE_20B/S.484K SP_236
FVNIQKEIDRLNEVA

SEQ ID NO: 332 - protein, 15 aa, Artificial Sequence
  SEQUENCE_21C SP_001
MFVFLVLLPLVSIQC

SEQ ID NO: 333 - protein, 15 aa, Artificial Sequence
  SEQUENCE_21C SP_002
VLLPLVSIQCVNLTT

SEQ ID NO: 334 - protein, 15 aa, Artificial Sequence
  SEQUENCE_21C SP_003
VSIQCVNLTTRTQLP

SEQ ID NO: 335 - protein, 15 aa, Artificial Sequence
  SEQUENCE_21C SP_029
LGVYYHKNNKSCMES

SEQ ID NO: 336 - protein, 15 aa, Artificial Sequence
  SEQUENCE_21C SP_030
HKNNKSCMESEFRVY

SEQ ID NO: 337 - protein, 15 aa, Artificial Sequence
  SEQUENCE_21C SP_031
SCMESEFRVYSSANN

SEQ ID NO: 338 - protein, 15 aa, Artificial Sequence
  SEQUENCE_21C SP_089
LDSKVGGNYNYRYRL

SEQ ID NO: 339 - protein, 15 aa, Artificial Sequence
  SEQUENCE_21C SP_090
GGNYNYRYRLFRKSN

SEQ ID NO: 340 - protein, 15 aa, Artificial Sequence
  SEQUENCE_21C SP_091
YRYRLFRKSNLKPFE

SEQ ID NO: 341 - protein, 15 aa, Artificial Sequence
  SEQUENCE_21C SP_121
GTNTSNQVAVLYQGV

SEQ ID NO: 342 - protein, 15 aa, Artificial Sequence
  SEQUENCE_21C SP_122
NQVAVLYQGVNCTEV

SEQ ID NO: 343 - protein, 15 aa, Artificial Sequence
  SEQUENCE_21C SP_123
LYQGVNCTEVPVAIH

SEQ ID NO: 344 - protein, 15 aa, Artificial Sequence
  SEQUENCE_21D SP_009
KVFRSSVLHSTRDLF

SEQ ID NO: 345 - protein, 15 aa, Artificial Sequence
  SEQUENCE_21D SP_010
SVLHSTRDLFLPFFS

SEQ ID NO: 346 - protein, 15 aa, Artificial Sequence
  SEQUENCE_21D SP_011
TRDLFLPFFSNVTWF

SEQ ID NO: 347 - protein, 15 aa, Artificial Sequence
  SEQUENCE_21D SP_012
LPFFSNVTWFHVISG

SEQ ID NO: 348 - protein, 15 aa, Artificial Sequence
  SEQUENCE_21D SP_013
NVTWFHVISGTNGTK

SEQ ID NO: 349 - protein, 15 aa, Artificial Sequence
  SEQUENCE_21D SP_014
HVISGTNGTKRFDNP

SEQ ID NO: 350 - protein, 15 aa, Artificial Sequence
  SEQUENCE_21D SP_027
CEFQFCNDPFLGVYH

SEQ ID NO: 351 - protein, 15 aa, Artificial Sequence
  SEQUENCE_21D SP_028
CNDPFLGVYHKNNKS

SEQ ID NO: 352 - protein, 15 aa, Artificial Sequence
  SEQUENCE_21D SP_029
LGVYHKNNKSWMESE

SEQ ID NO: 353 - protein, 15 aa, Artificial Sequence
  SEQUENCE_21D SP_095
EIYQAGSTPCNGVKG

SEQ ID NO: 354 - protein, 15 aa, Artificial Sequence
  SEQUENCE_21D SP_096
GSTPCNGVKGFNCYF

SEQ ID NO: 355 - protein, 15 aa, Artificial Sequence
  SEQUENCE_21D SP_097
NGVKGFNCYFPLQSY

SEQ ID NO: 356 - protein, 15 aa, Artificial Sequence
  SEQUENCE_21D SP_121
GTNTSNQVAVLYQGV

SEQ ID NO: 357 - protein, 15 aa, Artificial Sequence
  SEQUENCE_21D SP_122
NQVAVLYQGVNCTEV

SEQ ID NO: 358 - protein, 15 aa, Artificial Sequence
  SEQUENCE_21D SP_123
LYQGVNCTEVPVAIH

SEQ ID NO: 359 - protein, 15 aa, Artificial Sequence
  SEQUENCE_21D SP_134
IGAGICASYQTHTNS

SEQ ID NO: 360 - protein, 15 aa, Artificial Sequence
  SEQUENCE_21D SP_135
CASYQTHTNSPRRAR

SEQ ID NO: 361 - protein, 15 aa, Artificial Sequence
  SEQUENCE_21D SP_136
THTNSPRRARSVASQ

SEQ ID NO: 362 - protein, 15 aa, Artificial Sequence
  SEQUENCE_21D SP_176
ALLAGTITSGWTLGA

SEQ ID NO: 363 - protein, 15 aa, Artificial Sequence
  SEQUENCE_21D SP_177
TITSGWTLGAGAALQ

SEQ ID NO: 364 - protein, 15 aa, Artificial Sequence
  SEQUENCE_21D SP_178
WTLGAGAALQIPFAM

SEQ ID NO: 365 - protein, 15 aa, Artificial Sequence
  SEQUENCE_21F SP_001
MFVFFVLLPLVSSQC

SEQ ID NO: 366 - protein, 15 aa, Artificial Sequence
  SEQUENCE_21F SP_017
NPVLPFNDGVYFASI

SEQ ID NO: 367 - protein, 15 aa, Artificial Sequence
  SEQUENCE_21F SP_018
FNDGVYFASIEKSNI

SEQ ID NO: 368 - protein, 15 aa, Artificial Sequence
  SEQUENCE_21F SP_019
YFASIEKSNIIRGWI

SEQ ID NO: 369 - protein, 15 aa, Artificial Sequence
  SEQUENCE_21F SP_049
LLALHRSYLTPGGSS

SEQ ID NO: 370 - protein, 15 aa, Artificial Sequence
  SEQUENCE_21F SP_050
RSYLTPGGSSSGWTA

SEQ ID NO: 371 - protein, 15 aa, Artificial Sequence
  SEQUENCE_21F SP_051
PGGSSSGWTAGAAAY

SEQ ID NO: 372 - protein, 15 aa, Artificial Sequence
  SEQUENCE_21F SP_095
EIYQAGSTPCNGVKG

SEQ ID NO: 373 - protein, 15 aa, Artificial Sequence
  SEQUENCE_21F SP_096
GSTPCNGVKGFNCYF

SEQ ID NO: 374 - protein, 15 aa, Artificial Sequence
  SEQUENCE_21F SP_097
NGVKGFNCYFPLQSY

SEQ ID NO: 375 - protein, 15 aa, Artificial Sequence
  SEQUENCE_21F SP_121
GTNTSNQVAVLYQGV

SEQ ID NO: 376 - protein, 15 aa, Artificial Sequence
  SEQUENCE_21F SP_122
NQVAVLYQGVNCTEV

SEQ ID NO: 377 - protein, 15 aa, Artificial Sequence
  SEQUENCE_21F SP_123
LYQGVNCTEVPVAIH

SEQ ID NO: 378 - protein, 15 aa, Artificial Sequence
  SEQUENCE_21F SP_139
SIIAYTMSLGVENSV

SEQ ID NO: 379 - protein, 15 aa, Artificial Sequence
  SEQUENCE_21F SP_140
TMSLGVENSVAYSNN

SEQ ID NO: 380 - protein, 15 aa, Artificial Sequence
  SEQUENCE_21F SP_141
VENSVAYSNNSIAIP

SEQ ID NO: 381 - protein, 15 aa, Artificial Sequence
  SEQUENCE_21G SP_013
NVTWFHAIHVSGTNV

SEQ ID NO: 382 - protein, 15 aa, Artificial Sequence
  SEQUENCE_21G SP_014
HAIHVSGTNVIKRFD

SEQ ID NO: 383 - protein, 15 aa, Artificial Sequence
  SEQUENCE_21G SP_015
SGTNVIKRFDNPVLP

SEQ ID NO: 384 - protein, 15 aa, Artificial Sequence
  SEQUENCE_21G SP_016
IKRFDNPVLPFNDGV

SEQ ID NO: 385 - protein, 15 aa, Artificial Sequence
  SEQUENCE_21G SP_048
TRFQTLLALHNSSSG

SEQ ID NO: 386 - protein, 15 aa, Artificial Sequence
  SEQUENCE_21G SP_049
LLALHNSSSGWTAGA

SEQ ID NO: 387 - protein, 15 aa, Artificial Sequence
  SEQUENCE_21G SP_050
NSSSGWTAGAAAYYV

SEQ ID NO: 388 - protein, 15 aa, Artificial Sequence
  SEQUENCE_21G SP_089
LDSKVGGNYNYQYRL

SEQ ID NO: 389 - protein, 15 aa, Artificial Sequence
  SEQUENCE_21G SP_090
GGNYNYQYRLFRKSN

SEQ ID NO: 390 - protein, 15 aa, Artificial Sequence
  SEQUENCE_21G SP_091
YQYRLFRKSNLKPFE

SEQ ID NO: 391 - protein, 15 aa, Artificial Sequence
  SEQUENCE_21G SP_096
GSTPCNGVEGFNCYS

SEQ ID NO: 392 - protein, 15 aa, Artificial Sequence
  SEQUENCE_21G SP_097
NGVEGFNCYSPLQSY

SEQ ID NO: 393 - protein, 15 aa, Artificial Sequence
  SEQUENCE_21G SP_098
FNCYSPLQSYGFQPT

SEQ ID NO: 394 - protein, 15 aa, Artificial Sequence
  SEQUENCE_21G SP_121
GTNTSNQVAVLYQGV

SEQ ID NO: 395 - protein, 15 aa, Artificial Sequence
  SEQUENCE_21G SP_122
NQVAVLYQGVNCTEV

SEQ ID NO: 396 - protein, 15 aa, Artificial Sequence
  SEQUENCE_21G SP_123
LYQGVNCTEVPVAIH

SEQ ID NO: 397 - protein, 15 aa, Artificial Sequence
  SEQUENCE_21G SP_170
ARDLICAQKFNGLNV

SEQ ID NO: 398 - protein, 15 aa, Artificial Sequence
  SEQUENCE_21G SP_171
CAQKFNGLNVLPPLL

SEQ ID NO: 399 - protein, 15 aa, Artificial Sequence
  SEQUENCE_21G SP_172
NGLNVLPPLLTDEMI

SEQ ID NO: 400 - protein, 15 aa, Artificial Sequence
  SEQUENCE_21H SP_017
NPVLPFNDGVYFASI

SEQ ID NO: 401 - protein, 15 aa, Artificial Sequence
  SEQUENCE_21H SP_018
FNDGVYFASIEKSNI

SEQ ID NO: 402 - protein, 15 aa, Artificial Sequence
  SEQUENCE_21H SP_019
YFASIEKSNIIRGWI

SEQ ID NO: 403 - protein, 15 aa, Artificial Sequence
  SEQUENCE_21H SP_027
CEFQFCNDPFLGVSN

SEQ ID NO: 404 - protein, 15 aa, Artificial Sequence
  SEQUENCE_21H SP_028
CNDPFLGVSNHKNNK

SEQ ID NO: 405 - protein, 15 aa, Artificial Sequence
  SEQUENCE_21H SP_029
LGVSNHKNNKSWMES

SEQ ID NO: 406 - protein, 15 aa, Artificial Sequence
  SEQUENCE_21H SP_068
CPFGEVFNATKFASV

SEQ ID NO: 407 - protein, 15 aa, Artificial Sequence
  SEQUENCE_21H SP_069
VFNATKFASVYAWNR

SEQ ID NO: 408 - protein, 15 aa, Artificial Sequence
  SEQUENCE_21H SP_070
KFASVYAWNRKRISN

SEQ ID NO: 409 - protein, 15 aa, Artificial Sequence
  SEQUENCE_21H SP_095
EIYQAGSTPCNGVKG

SEQ ID NO: 410 - protein, 15 aa, Artificial Sequence
  SEQUENCE_21H SP_096
GSTPCNGVKGFNCYF

SEQ ID NO: 411 - protein, 15 aa, Artificial Sequence
  SEQUENCE_21H SP_097
NGVKGFNCYFPLQSY

SEQ ID NO: 412 - protein, 15 aa, Artificial Sequence
  SEQUENCE_21H SP_099
PLQSYGFQPTYGVGY

SEQ ID NO: 413 - protein, 15 aa, Artificial Sequence
  SEQUENCE_21H SP_100
GFQPTYGVGYQPYRV

SEQ ID NO: 414 - protein, 15 aa, Artificial Sequence
  SEQUENCE_21H SP_101
YGVGYQPYRVVVLSF

SEQ ID NO: 415 - protein, 15 aa, Artificial Sequence
  SEQUENCE_21H SP_121
GTNTSNQVAVLYQGV

SEQ ID NO: 416 - protein, 15 aa, Artificial Sequence
  SEQUENCE_21H SP_122
NQVAVLYQGVNCTEV

SEQ ID NO: 417 - protein, 15 aa, Artificial Sequence
  SEQUENCE_21H SP_123
LYQGVNCTEVPVAIH

SEQ ID NO: 418 - protein, 15 aa, Artificial Sequence
  SEQUENCE_21H SP_135
CASYQTQTNSHRRAR

SEQ ID NO: 419 - protein, 15 aa, Artificial Sequence
  SEQUENCE_21H SP_136
TQTNSHRRARSVASQ

SEQ ID NO: 420 - protein, 15 aa, Artificial Sequence
  SEQUENCE_21H SP_137
HRRARSVASQSIIAY

SEQ ID NO: 421 - protein, 15 aa, Artificial Sequence
  SEQUENCE_21H SP_188
DSLSSTASALGKLQN

SEQ ID NO: 422 - protein, 15 aa, Artificial Sequence
  SEQUENCE_21H SP_189
TASALGKLQNVVNQN

SEQ ID NO: 423 - protein, 15 aa, Artificial Sequence
  SEQUENCE_21H SP_190
GKLQNVVNQNAQALN

SEQ ID NO: 424 - protein, 15 aa, Artificial Sequence
  SEQUENCE_20A/S126A SP_004
VNLTTRTQLPSAYTN

SEQ ID NO: 425 - protein, 15 aa, Artificial Sequence
  SEQUENCE_20A/S126A SP_005
RTQLPSAYTNSFTRG

SEQ ID NO: 426 - protein, 15 aa, Artificial Sequence
  SEQUENCE_20A/S126A SP_006
SAYTNSFTRGVYYPD

SEQ ID NO: 427 - protein, 15 aa, Artificial Sequence
  SEQUENCE_20A/S126A SP_012
LPFFSNVTWFHAISG

SEQ ID NO: 428 - protein, 15 aa, Artificial Sequence
  SEQUENCE_20A/S126A SP_013
NVTWFHAISGTNGTK

SEQ ID NO: 429 - protein, 15 aa, Artificial Sequence
  SEQUENCE_20A/S126A SP_014
HAISGTNGTKRFDNP

SEQ ID NO: 430 - protein, 15 aa, Artificial Sequence
  SEQUENCE_20A/S126A SP_024
SLLIVNNATNAVIKV

SEQ ID NO: 431 - protein, 15 aa, Artificial Sequence
  SEQUENCE_20A/S126A SP_025
NNATNAVIKVCEFQF

SEQ ID NO: 432 - protein, 15 aa, Artificial Sequence
  SEQUENCE_20A/S126A SP_026
AVIKVCEFQFCNDPF

SEQ ID NO: 433 - protein, 15 aa, Artificial Sequence
  SEQUENCE_20A/S126A SP_027
CEFQFCNDPFLGVYH

SEQ ID NO: 434 - protein, 15 aa, Artificial Sequence
  SEQUENCE_20A/S126A SP_028
CNDPFLGVYHKNNKS

SEQ ID NO: 435 - protein, 15 aa, Artificial Sequence
  SEQUENCE_20A/S126A SP_029
LGVYHKNNKSWMESE

SEQ ID NO: 436 - protein, 15 aa, Artificial Sequence
  SEQUENCE_20A/S126A SP_047
IGINITRFQTLYRSY

SEQ ID NO: 437 - protein, 15 aa, Artificial Sequence
  SEQUENCE_20A/S126A SP_048
TRFQTLYRSYLTPGD

SEQ ID NO: 438 - protein, 15 aa, Artificial Sequence
  SEQUENCE_20A/S126A SP_049
LYRSYLTPGDSSSGW

SEQ ID NO: 439 - protein, 15 aa, Artificial Sequence
  SEQUENCE_20A/S126A SP_094
RDISTEIYQAGNTPC

SEQ ID NO: 440 - protein, 15 aa, Artificial Sequence
  SEQUENCE_20A/S126A SP_095
EIYQAGNTPCNGVKG

SEQ ID NO: 441 - protein, 15 aa, Artificial Sequence
  SEQUENCE_20A/S126A SP_096
GNTPCNGVKGFNCYF

SEQ ID NO: 442 - protein, 15 aa, Artificial Sequence
  SEQUENCE_20A/S126A SP_097
NGVKGFNCYFPLQSY

SEQ ID NO: 443 - protein, 15 aa, Artificial Sequence
  SEQUENCE_20A/S126A SP_121
GTNTSNQVAVLYQGV

SEQ ID NO: 444 - protein, 15 aa, Artificial Sequence
  SEQUENCE_20A/S126A SP_122
NQVAVLYQGVNCTEV

SEQ ID NO: 445 - protein, 15 aa, Artificial Sequence
  SEQUENCE_20A/S126A SP_123
LYQGVNCTEVPVAIH

SEQ ID NO: 446 - protein, 15 aa, Artificial Sequence
  SEQUENCE_20A/S126A SP_135
CASYQTQTNSHRRAR

SEQ ID NO: 447 - protein, 15 aa, Artificial Sequence
  SEQUENCE_20A/S126A SP_136
TQTNSHRRARSVASQ

SEQ ID NO: 448 - protein, 15 aa, Artificial Sequence
  SEQUENCE_20A/S126A SP_137
HRRARSVASQSIIAY

SEQ ID NO: 449 - protein, 15 aa, Artificial Sequence
  SEQUENCE_20A/S126A SP_204
AEIRASANLAAIKMS

SEQ ID NO: 450 - protein, 15 aa, Artificial Sequence
  SEQUENCE_20A/S126A SP_205
SANLAAIKMSECVLG

SEQ ID NO: 451 - protein, 15 aa, Artificial Sequence
  SEQUENCE_20A/S126A SP_206
AIKMSECVLGQSKRV

SEQ ID NO: 452 - protein, 15 aa, Artificial Sequence
  SEQUENCE_20A/S126A SP_222
QRNFYEPQIITTHNT

SEQ ID NO: 453 - protein, 15 aa, Artificial Sequence
  SEQUENCE_20A/S126A SP_223
EPQIITTHNTFVSGN

SEQ ID NO: 454 - protein, 15 aa, Artificial Sequence
  SEQUENCE_20A/S126A SP_224
TTHNTFVSGNCDVVI

SEQ ID NO: 455 - DNA, 21 bp, Artificial Sequence
  ACTIN probe
tcatccatggtgagctggcgg

SEQ ID NO: 456 - DNA, 27 bp, Artificial Sequence
  CXCL10 probe
agtggcattcaaggagtacctctctct

SEQ ID NO: 457 - DNA, 17 bp, Artificial Sequence
  ACTIN primer 1
ccttgcacatgccggag

SEQ ID NO: 458 - DNA, 17 bp, Artificial Sequence
  ACTIN primer 2
acagagcctcgcctttg

SEQ ID NO: 459 - DNA, 23 bp, Artificial Sequence
  CXCL10 primer 1
ccattctgatttgctgccttatc

SEQ ID NO: 460 - DNA, 23 bp, Artificial Sequence
  CXCL10 primer 2
tactaatgctgatgcaggtacag

SEQ ID NO: 461 - protein, 15 aa, Artificial Sequence
  Wildtype SP_002
VLLPLVSSQCVNLTT

SEQ ID NO: 462 - protein, 15 aa, Artificial Sequence
  Wildtype SP_003
VSSQCVNLTTRTQLP

SEQ ID NO: 463 - protein, 15 aa, Artificial Sequence
  Wildtype SP_004
VNLTTRTQLPPAYTN

SEQ ID NO: 464 - protein, 15 aa, Artificial Sequence
  Wildtype SP_027
CEFQFCNDPFLGVYY

SEQ ID NO: 465 - protein, 15 aa, Artificial Sequence
  Wildtype SP_028
CNDPFLGVYYHKNNK

SEQ ID NO: 466 - protein, 15 aa, Artificial Sequence
  Wildtype SP_029
LGVYYHKNNKSWMES

SEQ ID NO: 467 - protein, 15 aa, Artificial Sequence
  Wildtype SP_030
HKNNKSWMESEFRVY

SEQ ID NO: 468 - protein, 15 aa, Artificial Sequence
  Wildtype SP_031
SWMESEFRVYSSANN

SEQ ID NO: 469 - protein, 15 aa, Artificial Sequence
  Wildtype SP_032
EFRVYSSANNCTFEY

SEQ ID NO: 470 - protein, 15 aa, Artificial Sequence
  Wildtype SP_094
RDISTEIYQAGSTPC

SEQ ID NO: 471 - protein, 15 aa, Artificial Sequence
  Wildtype SP_095
EIYQAGSTPCNGVEG

SEQ ID NO: 472 - protein, 15 aa, Artificial Sequence
  Wildtype SP_096
GSTPCNGVEGFNCYF

SEQ ID NO: 473 - protein, 15 aa, Artificial Sequence
  Wildtype SP_121
GTNTSNQVAVLYQDV

SEQ ID NO: 474 - protein, 15 aa, Artificial Sequence
  Wildtype SP_122
NQVAVLYQDVNCTEV

SEQ ID NO: 475 - protein, 15 aa, Artificial Sequence
  Wildtype SP_123
LYQDVNCTEVPVAIH

SEQ ID NO: 476 - protein, 15 aa, Artificial Sequence
  Wildtype SP_135
CASYQTQTNSPRRAR

SEQ ID NO: 477 - protein, 15 aa, Artificial Sequence
  Wildtype SP_136
TQTNSPRRARSVASQ

SEQ ID NO: 478 - protein, 15 aa, Artificial Sequence
  Wildtype SP_137
PRRARSVASQSIIAY

SEQ ID NO: 479 - protein, 15 aa, Artificial Sequence
  Wildtype SP_190
GKLQDVVNQNAQALN

SEQ ID NO: 480 - protein, 15 aa, Artificial Sequence
  HBV C_1
MQLFHLCLIISCLRP

SEQ ID NO: 481 - protein, 15 aa, Artificial Sequence
  HBV C_2
LCLIISCLRPTVQAS

SEQ ID NO: 482 - protein, 15 aa, Artificial Sequence
  HBV C_3
SCLRPTVQASKLCLG

SEQ ID NO: 483 - protein, 15 aa, Artificial Sequence
  HBV C_4
TVQASKLCLGWLWGM

SEQ ID NO: 484 - protein, 15 aa, Artificial Sequence
  HBV C_5
KLCLGWLWGMDIDPY

SEQ ID NO: 485 - protein, 15 aa, Artificial Sequence
  HBV C_6
WLWGMDIDPYKEFGA

SEQ ID NO: 486 - protein, 15 aa, Artificial Sequence
  HBV C_7
DIDPYKEFGASVELL

SEQ ID NO: 487 - protein, 15 aa, Artificial Sequence
  HBV C_8
KEFGASVELLSFLPS

SEQ ID NO: 488 - protein, 15 aa, Artificial Sequence
  HBV C_9
SVELLSFLPSDFFPN

SEQ ID NO: 489 - protein, 15 aa, Artificial Sequence
  HBV C_10
SFLPSDFFPNIRDLL

SEQ ID NO: 490 - protein, 15 aa, Artificial Sequence
  HBV C_11
DFFPNIRDLLDTASA

SEQ ID NO: 491 - protein, 15 aa, Artificial Sequence
  HBV C_12
IRDLLDTASALFREA

SEQ ID NO: 492 - protein, 15 aa, Artificial Sequence
  HBV C_13
DTASALFREALESPE

SEQ ID NO: 493 - protein, 15 aa, Artificial Sequence
  HBV C_14
LFREALESPEHCTPH

SEQ ID NO: 494 - protein, 15 aa, Artificial Sequence
  HBV C_15
LESPEHCTPHHTAIR

SEQ ID NO: 495 - protein, 15 aa, Artificial Sequence
  HBV C_16
HCTPHHTAIRQAILC

SEQ ID NO: 496 - protein, 15 aa, Artificial Sequence
  HBV C_17
HTAIRQAILCWGELM

SEQ ID NO: 497 - protein, 15 aa, Artificial Sequence
  HBV C_18
QAILCWGELMNLATW

SEQ ID NO: 498 - protein, 15 aa, Artificial Sequence
  HBV C_19
WGELMNLATWVGSNL

SEQ ID NO: 499 - protein, 15 aa, Artificial Sequence
  HBV C_20
NLATWVGSNLEDPAS

SEQ ID NO: 500 - protein, 15 aa, Artificial Sequence
  HBV C_21
VGSNLEDPASRELVV

SEQ ID NO: 501 - protein, 15 aa, Artificial Sequence
  HBV C_22
EDPASRELVVGYVNV

SEQ ID NO: 502 - protein, 15 aa, Artificial Sequence
  HBV C_23
RELVVGYVNVNMGLK

SEQ ID NO: 503 - protein, 15 aa, Artificial Sequence
  HBV C_24
GYVNVNMGLKIRQLL

SEQ ID NO: 504 - protein, 15 aa, Artificial Sequence
  HBV C_25
NMGLKIRQLLWFHIS

SEQ ID NO: 505 - protein, 15 aa, Artificial Sequence
  HBV C_26
IRQLLWFHISCLTFG

SEQ ID NO: 506 - protein, 15 aa, Artificial Sequence
  HBV C_27
WFHISCLTFGRETVL

SEQ ID NO: 507 - protein, 15 aa, Artificial Sequence
  HBV C_28
CLTFGRETVLEYLVS

SEQ ID NO: 508 - protein, 15 aa, Artificial Sequence
  HBV C_29
RETVLEYLVSFGVWI

SEQ ID NO: 509 - protein, 15 aa, Artificial Sequence
  HBV C_30
EYLVSFGVWIRTPPA

SEQ ID NO: 510 - protein, 15 aa, Artificial Sequence
  HBV C_31
FGVWIRTPPAYRPPN

SEQ ID NO: 511 - protein, 15 aa, Artificial Sequence
  HBV C_32
RTPPAYRPPNAPILS

SEQ ID NO: 512 - protein, 15 aa, Artificial Sequence
  HBV C_33
YRPPNAPILSTLPET

SEQ ID NO: 513 - protein, 15 aa, Artificial Sequence
  HBV C_34
APILSTLPETTVVRR

SEQ ID NO: 514 - protein, 15 aa, Artificial Sequence
  HBV C_35
TLPETTVVRRRGRSP

SEQ ID NO: 515 - protein, 15 aa, Artificial Sequence
  HBV C_36
TVVRRRGRSPRRRTP

SEQ ID NO: 516 - protein, 15 aa, Artificial Sequence
  HBV C_37
RGRSPRRRTPSPRRR

SEQ ID NO: 517 - protein, 15 aa, Artificial Sequence
  HBV C_38
RRRTPSPRRRRSQSP

SEQ ID NO: 518 - protein, 15 aa, Artificial Sequence
  HBV C_39
SPRRRRSQSPRRRRS

SEQ ID NO: 519 - protein, 15 aa, Artificial Sequence
  HBV C_40
RSQSPRRRRSQSRGS

SEQ ID NO: 520 - protein, 12 aa, Artificial Sequence
  HBV C_41
RRRRSQSRGSQC

SEQ ID NO: 521 - protein, 15 aa, Artificial Sequence
  HBV X_1
MAARLCCQLDPARDV

SEQ ID NO: 522 - protein, 15 aa, Artificial Sequence
  HBV X_2
CCQLDPARDVLCLRP

SEQ ID NO: 523 - protein, 15 aa, Artificial Sequence
  HBV X_3
PARDVLCLRPVGAES

SEQ ID NO: 524 - protein, 15 aa, Artificial Sequence
  HBV X_4
LCLRPVGAESRGRPV

SEQ ID NO: 525 - protein, 15 aa, Artificial Sequence
  HBV X_5
VGAESRGRPVSGAFG

SEQ ID NO: 526 - protein, 15 aa, Artificial Sequence
  HBV X_6
RGRPVSGAFGTLPSP

SEQ ID NO: 527 - protein, 15 aa, Artificial Sequence
  HBV X_7
SGAFGTLPSPSSSAV

SEQ ID NO: 528 - protein, 15 aa, Artificial Sequence
  HBV X_8
TLPSPSSSAVPTDHG

SEQ ID NO: 529 - protein, 15 aa, Artificial Sequence
  HBV X_9
SSSAVPTDHGAHLSL

SEQ ID NO: 530 - protein, 15 aa, Artificial Sequence
  HBV X_10
PTDHGAHLSLRGLPV

SEQ ID NO: 531 - protein, 15 aa, Artificial Sequence
  HBV X_11
AHLSLRGLPVCAFSS

SEQ ID NO: 532 - protein, 15 aa, Artificial Sequence
  HBV X_12
RGLPVCAFSSAGPCA

SEQ ID NO: 533 - protein, 15 aa, Artificial Sequence
  HBV X_13
CAFSSAGPCALRFTS

SEQ ID NO: 534 - protein, 15 aa, Artificial Sequence
  HBV X_14
AGPCALRFTSARRME

SEQ ID NO: 535 - protein, 15 aa, Artificial Sequence
  HBV X_15
LRFTSARRMETTVNA

SEQ ID NO: 536 - protein, 15 aa, Artificial Sequence
  HBV X_16
ARRMETTVNARQVLP

SEQ ID NO: 537 - protein, 15 aa, Artificial Sequence
  HBV X_17
TTVNARQVLPKVLHK

SEQ ID NO: 538 - protein, 15 aa, Artificial Sequence
  HBV X_18
RQVLPKVLHKRTLGL

SEQ ID NO: 539 - protein, 15 aa, Artificial Sequence
  HBV X_19
KVLHKRTLGLSAMST

SEQ ID NO: 540 - protein, 15 aa, Artificial Sequence
  HBV X_20
RTLGLSAMSTTDLEA

SEQ ID NO: 541 - protein, 15 aa, Artificial Sequence
  HBV X_21
SAMSTTDLEAYFKDR

SEQ ID NO: 542 - protein, 15 aa, Artificial Sequence
  HBV X_22
TDLEAYFKDRVFKDW

SEQ ID NO: 543 - protein, 15 aa, Artificial Sequence
  HBV X_23
YFKDRVFKDWEELGE

SEQ ID NO: 544 - protein, 15 aa, Artificial Sequence
  HBV X_24
VFKDWEELGEETRLM

SEQ ID NO: 545 - protein, 15 aa, Artificial Sequence
  HBV X_25
EELGEETRLMIFVLG

SEQ ID NO: 546 - protein, 15 aa, Artificial Sequence
  HBV X_26
ETRLMIFVLGGCRHK

SEQ ID NO: 547 - protein, 15 aa, Artificial Sequence
  HBV X_27
IFVLGGCRHKLVCSP

SEQ ID NO: 548 - protein, 15 aa, Artificial Sequence
  HBV X_28
GCRHKLVCSPAPCNF

SEQ ID NO: 549 - protein, 14 aa, Artificial Sequence
  HBV X_29
LVCSPAPCNFFTSA

SEQ ID NO: 550 - protein, 15 aa, Artificial Sequence
  HBV E_1
MGTNLSVPNPLGFFP

SEQ ID NO: 551 - protein, 15 aa, Artificial Sequence
  HBV E_2
SVPNPLGFFPDHQLD

SEQ ID NO: 552 - protein, 15 aa, Artificial Sequence
  HBV E_3
LGFFPDHQLDPAFGA

SEQ ID NO: 553 - protein, 15 aa, Artificial Sequence
  HBV E_4
DHQLDPAFGANSNNP

SEQ ID NO: 554 - protein, 15 aa, Artificial Sequence
  HBV E_5
PAFGANSNNPDWDFN

SEQ ID NO: 555 - protein, 15 aa, Artificial Sequence
  HBV E_6
NSNNPDWDFNPNKDQ

SEQ ID NO: 556 - protein, 15 aa, Artificial Sequence
  HBV E_7
DWDFNPNKDQWPAAN

SEQ ID NO: 557 - protein, 15 aa, Artificial Sequence
  HBV E_8
PNKDQWPAANQVGVG

SEQ ID NO: 558 - protein, 15 aa, Artificial Sequence
  HBV E_9
WPAANQVGVGSFGPG

SEQ ID NO: 559 - protein, 15 aa, Artificial Sequence
  HBV E_10
QVGVGSFGPGFTPPH

SEQ ID NO: 560 - protein, 15 aa, Artificial Sequence
  HBV E_11
SFGPGFTPPHGNLLG

SEQ ID NO: 561 - protein, 15 aa, Artificial Sequence
  HBV E_12
FTPPHGNLLGWSPQA

SEQ ID NO: 562 - protein, 15 aa, Artificial Sequence
  HBV E_13
GNLLGWSPQAQGILT

SEQ ID NO: 563 - protein, 15 aa, Artificial Sequence
  HBV E_14
WSPQAQGILTTVPAA

SEQ ID NO: 564 - protein, 15 aa, Artificial Sequence
  HBV E_15
QGILTTVPAAPPPAS

SEQ ID NO: 565 - protein, 15 aa, Artificial Sequence
  HBV E_16
TVPAAPPPASTNRQS

SEQ ID NO: 566 - protein, 15 aa, Artificial Sequence
  HBV E_17
PPPASTNRQSGRQPT

SEQ ID NO: 567 - protein, 15 aa, Artificial Sequence
  HBV E_18
TNRQSGRQPTPISLP

SEQ ID NO: 568 - protein, 15 aa, Artificial Sequence
  HBV E_19
GRQPTPISLPLRDSH

SEQ ID NO: 569 - protein, 15 aa, Artificial Sequence
  HBV E_20
PISLPLRDSHPQAMQ

SEQ ID NO: 570 - protein, 15 aa, Artificial Sequence
  HBV E_21
LRDSHPQAMQWNSST

SEQ ID NO: 571 - protein, 15 aa, Artificial Sequence
  HBV E_22
PQAMQWNSSTFHQAL

SEQ ID NO: 572 - protein, 15 aa, Artificial Sequence
  HBV E_23
WNSSTFHQALLDPKV

SEQ ID NO: 573 - protein, 15 aa, Artificial Sequence
  HBV E_24
FHQALLDPKVRGLYL

SEQ ID NO: 574 - protein, 15 aa, Artificial Sequence
  HBV E_25
LDPKVRGLYLPAGGS

SEQ ID NO: 575 - protein, 15 aa, Artificial Sequence
  HBV E_26
RGLYLPAGGSSSGTV

SEQ ID NO: 576 - protein, 15 aa, Artificial Sequence
  HBV E_27
PAGGSSSGTVNPVQT

SEQ ID NO: 577 - protein, 15 aa, Artificial Sequence
  HBV E_28
SSGTVNPVQTTASPI

SEQ ID NO: 578 - protein, 15 aa, Artificial Sequence
  HBV E_29
NPVQTTASPISSIFS

SEQ ID NO: 579 - protein, 15 aa, Artificial Sequence
  HBV E_30
TASPISSIFSRTGDP

SEQ ID NO: 580 - protein, 15 aa, Artificial Sequence
  HBV E_31
SSIFSRTGDPAPNME

SEQ ID NO: 581 - protein, 15 aa, Artificial Sequence
  HBV E_32
RTGDPAPNMESTTSG

SEQ ID NO: 582 - protein, 15 aa, Artificial Sequence
  HBV E_33
APNMESTTSGFLGPL

SEQ ID NO: 583 - protein, 15 aa, Artificial Sequence
  HBV E_34
STTSGFLGPLLVLQA

SEQ ID NO: 584 - protein, 15 aa, Artificial Sequence
  HBV E_35
FLGPLLVLQAGFFLL

SEQ ID NO: 585 - protein, 15 aa, Artificial Sequence
  HBV E_36
LVLQAGFFLLTRILT

SEQ ID NO: 586 - protein, 15 aa, Artificial Sequence
  HBV E_37
GFFLLTRILTIPQSL

SEQ ID NO: 587 - protein, 15 aa, Artificial Sequence
  HBV E_38
TRILTIPQSLDSWWT

SEQ ID NO: 588 - protein, 15 aa, Artificial Sequence
  HBV E_39
IPQSLDSWWTSLNFL

SEQ ID NO: 589 - protein, 15 aa, Artificial Sequence
  HBV E_40
DSWWTSLNFLGGAPT

SEQ ID NO: 590 - protein, 15 aa, Artificial Sequence
  HBV E_41
SLNFLGGAPTCSGQN

SEQ ID NO: 591 - protein, 15 aa, Artificial Sequence
  HBV E_42
GGAPTCSGQNLQSPT

SEQ ID NO: 592 - protein, 15 aa, Artificial Sequence
  HBV E_43
CSGQNLQSPTSNHSP

SEQ ID NO: 593 - protein, 15 aa, Artificial Sequence
  HBV E_44
LQSPTSNHSPTSCPP

SEQ ID NO: 594 - protein, 15 aa, Artificial Sequence
  HBV E_45
SNHSPTSCPPICPGY

SEQ ID NO: 595 - protein, 15 aa, Artificial Sequence
  HBV E_46
TSCPPICPGYRWMCL

SEQ ID NO: 596 - protein, 15 aa, Artificial Sequence
  HBV E_47
ICPGYRWMCLRRFII

SEQ ID NO: 597 - protein, 15 aa, Artificial Sequence
  HBV E_48
RWMCLRRFIIFLFIL

SEQ ID NO: 598 - protein, 15 aa, Artificial Sequence
  HBV E_49
RRFIIFLFILLLCLI

SEQ ID NO: 599 - protein, 15 aa, Artificial Sequence
  HBV E_50
FLFILLLCLIFLLVL

SEQ ID NO: 600 - protein, 15 aa, Artificial Sequence
  HBV E_51
LLCLIFLLVLLDYQG

SEQ ID NO: 601 - protein, 15 aa, Artificial Sequence
  HBV E_52
FLLVLLDYQGMLPVC

SEQ ID NO: 602 - protein, 15 aa, Artificial Sequence
  HBV E_53
LDYQGMLPVCPLLPG

SEQ ID NO: 603 - protein, 15 aa, Artificial Sequence
  HBV E_54
MLPVCPLLPGTSTTS

SEQ ID NO: 604 - protein, 15 aa, Artificial Sequence
  HBV E_55
PLLPGTSTTSMGPCK

SEQ ID NO: 605 - protein, 15 aa, Artificial Sequence
  HBV E_56
TSTTSMGPCKTCTTP

SEQ ID NO: 606 - protein, 15 aa, Artificial Sequence
  HBV E_57
MGPCKTCTTPAQGTS

SEQ ID NO: 607 - protein, 15 aa, Artificial Sequence
  HBV E_58
TCTTPAQGTSMFPSC

SEQ ID NO: 608 - protein, 15 aa, Artificial Sequence
  HBV E_59
AQGTSMFPSCCCTQP

SEQ ID NO: 609 - protein, 15 aa, Artificial Sequence
  HBV E_60
MFPSCCCTQPSDGNC

SEQ ID NO: 610 - protein, 15 aa, Artificial Sequence
  HBV E_61
CCTQPSDGNCTCIPI

SEQ ID NO: 611 - protein, 15 aa, Artificial Sequence
  HBV E_62
SDGNCTCIPIPSSWA

SEQ ID NO: 612 - protein, 15 aa, Artificial Sequence
  HBV E_63
TCIPIPSSWAFARFL

SEQ ID NO: 613 - protein, 15 aa, Artificial Sequence
  HBV E_64
PSSWAFARFLWEWAS

SEQ ID NO: 614 - protein, 15 aa, Artificial Sequence
  HBV E_65
FARFLWEWASVRFSW

SEQ ID NO: 615 - protein, 15 aa, Artificial Sequence
  HBV E_66
WEWASVRFSWLSLLV

SEQ ID NO: 616 - protein, 15 aa, Artificial Sequence
  HBV E_67
VRFSWLSLLVPFVQW

SEQ ID NO: 617 - protein, 15 aa, Artificial Sequence
  HBV E_68
LSLLVPFVQWFVGLS

SEQ ID NO: 618 - protein, 15 aa, Artificial Sequence
  HBV E_69
PFVQWFVGLSPTVWL

SEQ ID NO: 619 - protein, 15 aa, Artificial Sequence
  HBV E_70
FVGLSPTVWLSVIWM

SEQ ID NO: 620 - protein, 15 aa, Artificial Sequence
  HBV E_71
PTVWLSVIWMMWYWG

SEQ ID NO: 621 - protein, 15 aa, Artificial Sequence
  HBV E_72
SVIWMMWYWGRSLYN

SEQ ID NO: 622 - protein, 15 aa, Artificial Sequence
  HBV E_73
MWYWGRSLYNILNPF

SEQ ID NO: 623 - protein, 15 aa, Artificial Sequence
  HBV E_74
RSLYNILNPFLPLLP

SEQ ID NO: 624 - protein, 15 aa, Artificial Sequence
  HBV E_75
ILNPFLPLLPIFFCL

SEQ ID NO: 625 - protein, 14 aa, Artificial Sequence
  HBV E_76
LPLLPIFFCLWVYI

SEQ ID NO: 626 - protein, 15 aa, Artificial Sequence
  HBV Pol_1
MPLSYQHFRKLLLLD

SEQ ID NO: 627 - protein, 15 aa, Artificial Sequence
  HBV Pol_2
QHFRKLLLLDDEAGP

SEQ ID NO: 628 - protein, 15 aa, Artificial Sequence
  HBV Pol_4
LLLLDDEAGPLEEEL

SEQ ID NO: 629 - protein, 15 aa, Artificial Sequence
  HBV Pol_4
DEAGPLEEELPRLAD

SEQ ID NO: 630 - protein, 15 aa, Artificial Sequence
  HBV Pol_5
LEEELPRLADEGLNH

SEQ ID NO: 631 - protein, 15 aa, Artificial Sequence
  HBV Pol_6
PRLADEGLNHRVAED

SEQ ID NO: 632 - protein, 15 aa, Artificial Sequence
  HBV Pol_7
EGLNHRVAEDLNLGD

SEQ ID NO: 633 - protein, 15 aa, Artificial Sequence
  HBV Pol_8
RVAEDLNLGDLNVSI

SEQ ID NO: 634 - protein, 15 aa, Artificial Sequence
  HBV Pol_9
LNLGDLNVSIPWTHK

SEQ ID NO: 635 - protein, 15 aa, Artificial Sequence
  HBV Pol_10
LNVSIPWTHKVGNFT

SEQ ID NO: 636 - protein, 15 aa, Artificial Sequence
  HBV Pol_11
PWTHKVGNFTGLYSS

SEQ ID NO: 637 - protein, 15 aa, Artificial Sequence
  HBV Pol_12
VGNFTGLYSSTVPVF

SEQ ID NO: 638 - protein, 15 aa, Artificial Sequence
  HBV Pol_13
GLYSSTVPVFNPEWK

SEQ ID NO: 639 - protein, 15 aa, Artificial Sequence
  HBV Pol_14
TVPVFNPEWKTPSFP

SEQ ID NO: 640 - protein, 15 aa, Artificial Sequence
  HBV Pol_15
NPEWKTPSFPNIHLK

SEQ ID NO: 641 - protein, 15 aa, Artificial Sequence
  HBV Pol_16
TPSFPNIHLKEDIIN

SEQ ID NO: 642 - protein, 15 aa, Artificial Sequence
  HBV Pol_17
NIHLKEDIINRCQQY

SEQ ID NO: 643 - protein, 15 aa, Artificial Sequence
  HBV Pol_18
EDIINRCQQYVGPLT

SEQ ID NO: 644 - protein, 15 aa, Artificial Sequence
  HBV Pol_19
RCQQYVGPLTVNEKR

SEQ ID NO: 645 - protein, 15 aa, Artificial Sequence
  HBV Pol_20
VGPLTVNEKRRLKVT

SEQ ID NO: 646 - protein, 15 aa, Artificial Sequence
  HBV Pol_21
VNEKRRLKVTMPARF

SEQ ID NO: 647 - protein, 15 aa, Artificial Sequence
  HBV Pol_22
RLKVTMPARFYPNLT

SEQ ID NO: 648 - protein, 15 aa, Artificial Sequence
  HBV Pol_23
MPARFYPNLTKYLPL

SEQ ID NO: 649 - protein, 15 aa, Artificial Sequence
  HBV Pol_24
YPNLTKYLPLDKGIK

SEQ ID NO: 650 - protein, 15 aa, Artificial Sequence
  HBV Pol_25
KYLPLDKGIKPYYPE

SEQ ID NO: 651 - protein, 15 aa, Artificial Sequence
  HBV Pol_26
DKGIKPYYPEHIVNH

SEQ ID NO: 652 - protein, 15 aa, Artificial Sequence
  HBV Pol_27
PYYPEHIVNHYFQTR

SEQ ID NO: 653 - protein, 15 aa, Artificial Sequence
  HBV Pol_28
HIVNHYFQTRHYLHT

SEQ ID NO: 654 - protein, 15 aa, Artificial Sequence
  HBV Pol_29
YFQTRHYLHTLWKAG

SEQ ID NO: 655 - protein, 15 aa, Artificial Sequence
  HBV Pol_30
HYLHTLWKAGILYKR

SEQ ID NO: 656 - protein, 15 aa, Artificial Sequence
  HBV Pol_31
LWKAGILYKRETTRS

SEQ ID NO: 657 - protein, 15 aa, Artificial Sequence
  HBV Pol_32
ILYKRETTRSASFCG

SEQ ID NO: 658 - protein, 15 aa, Artificial Sequence
  HBV Pol_33
ETTRSASFCGSPYSW

SEQ ID NO: 659 - protein, 15 aa, Artificial Sequence
  HBV Pol_34
ASFCGSPYSWEQELQ

SEQ ID NO: 660 - protein, 15 aa, Artificial Sequence
  HBV Pol_35
SPYSWEQELQHGTLV

SEQ ID NO: 661 - protein, 15 aa, Artificial Sequence
  HBV Pol_36
EQELQHGTLVFQTST

SEQ ID NO: 662 - protein, 15 aa, Artificial Sequence
  HBV Pol_37
HGTLVFQTSTRHGDE

SEQ ID NO: 663 - protein, 15 aa, Artificial Sequence
  HBV Pol_38
FQTSTRHGDESFGSQ

SEQ ID NO: 664 - protein, 15 aa, Artificial Sequence
  HBV Pol_39
RHGDESFGSQSSGIL

SEQ ID NO: 665 - protein, 15 aa, Artificial Sequence
  HBV Pol_40
SFGSQSSGILSRSPV

SEQ ID NO: 666 - protein, 15 aa, Artificial Sequence
  HBV Pol_41
SSGILSRSPVGPGIR

SEQ ID NO: 667 - protein, 15 aa, Artificial Sequence
  HBV Pol_42
SRSPVGPGIRSQFKQ

SEQ ID NO: 668 - protein, 15 aa, Artificial Sequence
  HBV Pol_43
GPGIRSQFKQSRLGL

SEQ ID NO: 669 - protein, 15 aa, Artificial Sequence
  HBV Pol_44
SQFKQSRLGLQPQQG

SEQ ID NO: 670 - protein, 15 aa, Artificial Sequence
  HBV Pol_45
SRLGLQPQQGSMASG

SEQ ID NO: 671 - protein, 15 aa, Artificial Sequence
  HBV Pol_46
SRLGLQPQQGSMASG

SEQ ID NO: 672 - protein, 15 aa, Artificial Sequence
  HBV Pol_47
SMASGKPGRSGIIRA

SEQ ID NO: 673 - protein, 15 aa, Artificial Sequence
  HBV Pol_48
KPGRSGIIRARVHPT

SEQ ID NO: 674 - protein, 15 aa, Artificial Sequence
  HBV Pol_49
GIIRARVHPTTRQSF

SEQ ID NO: 675 - protein, 15 aa, Artificial Sequence
  HBV Pol_50
RVHPTTRQSFGVEPA

SEQ ID NO: 676 - protein, 15 aa, Artificial Sequence
  HBV Pol_51
TRQSFGVEPAGSGHI

SEQ ID NO: 677 - protein, 15 aa, Artificial Sequence
  HBV Pol_52
GVEPAGSGHIDNSTS

SEQ ID NO: 678 - protein, 15 aa, Artificial Sequence
  HBV Pol_53
GSGHIDNSTSSASSC

SEQ ID NO: 679 - protein, 15 aa, Artificial Sequence
  HBV Pol_54
DNSTSSASSCLHQSA

SEQ ID NO: 680 - protein, 15 aa, Artificial Sequence
  HBV Pol_55
SASSCLHQSAVRKTA

SEQ ID NO: 681 - protein, 15 aa, Artificial Sequence
  HBV Pol_56
LHQSAVRKTAYSHLS

SEQ ID NO: 682 - protein, 15 aa, Artificial Sequence
  HBV Pol_57
VRKTAYSHLSTSKRQ

SEQ ID NO: 683 - protein, 15 aa, Artificial Sequence
  HBV Pol_58
YSHLSTSKRQSSSGH

SEQ ID NO: 684 - protein, 15 aa, Artificial Sequence
  HBV Pol_59
TSKRQSSSGHAVELQ

SEQ ID NO: 685 - protein, 15 aa, Artificial Sequence
  HBV Pol_60
SSSGHAVELQHIPPS

SEQ ID NO: 686 - protein, 15 aa, Artificial Sequence
  HBV Pol_61
AVELQHIPPSSARSQ

SEQ ID NO: 687 - protein, 15 aa, Artificial Sequence
  HBV Pol_62
HIPPSSARSQSEGPI

SEQ ID NO: 688 - protein, 15 aa, Artificial Sequence
  HBV Pol_63
SARSQSEGPIPSCWW

SEQ ID NO: 689 - protein, 15 aa, Artificial Sequence
  HBV Pol_64
SEGPIPSCWWLQFRN

SEQ ID NO: 690 - protein, 15 aa, Artificial Sequence
  HBV Pol_65
PSCWWLQFRNSKPCS

SEQ ID NO: 691 - protein, 15 aa, Artificial Sequence
  HBV Pol_66
LQFRNSKPCSDYCLS

SEQ ID NO: 692 - protein, 15 aa, Artificial Sequence
  HBV Pol_67
SKPCSDYCLSHIVNL

SEQ ID NO: 693 - protein, 15 aa, Artificial Sequence
  HBV Pol_68
DYCLSHIVNLLEDWG

SEQ ID NO: 694 - protein, 15 aa, Artificial Sequence
  HBV Pol_69
HIVNLLEDWGPCTEY

SEQ ID NO: 695 - protein, 15 aa, Artificial Sequence
  HBV Pol_70
LEDWGPCTEYGEHHI

SEQ ID NO: 696 - protein, 15 aa, Artificial Sequence
  HBV Pol_71
PCTEYGEHHIRIPRT

SEQ ID NO: 697 - protein, 15 aa, Artificial Sequence
  HBV Pol_72
GEHHIRIPRTPARVT

SEQ ID NO: 698 - protein, 15 aa, Artificial Sequence
  HBV Pol_73
RIPRTPARVTGGVFL

SEQ ID NO: 699 - protein, 15 aa, Artificial Sequence
  HBV Pol_74
PARVTGGVFLVDKNP

SEQ ID NO: 700 - protein, 15 aa, Artificial Sequence
  HBV Pol_75
GGVFLVDKNPHNTTE

SEQ ID NO: 701 - protein, 15 aa, Artificial Sequence
  HBV Pol_76
VDKNPHNTTESRLVV

SEQ ID NO: 702 - protein, 15 aa, Artificial Sequence
  HBV Pol_77
HNTTESRLVVDFSQF

SEQ ID NO: 703 - protein, 15 aa, Artificial Sequence
  HBV Pol_78
SRLVVDFSQFSRGST

SEQ ID NO: 704 - protein, 15 aa, Artificial Sequence
  HBV Pol_79
DFSQFSRGSTHVFWP

SEQ ID NO: 705 - protein, 15 aa, Artificial Sequence
  HBV Pol_80
SRGSTHVFWPKFAVP

SEQ ID NO: 706 - protein, 15 aa, Artificial Sequence
  HBV Pol_81
HVFWPKFAVPNLQSL

SEQ ID NO: 707 - protein, 15 aa, Artificial Sequence
  HBV Pol_82
KFAVPNLQSLTNLLS

SEQ ID NO: 708 - protein, 15 aa, Artificial Sequence
  HBV Pol_83
NLQSLTNLLSSNLSW

SEQ ID NO: 709 - protein, 15 aa, Artificial Sequence
  HBV Pol_84
TNLLSSNLSWLSLDV

SEQ ID NO: 710 - protein, 15 aa, Artificial Sequence
  HBV Pol_85
SNLSWLSLDVSAAFY

SEQ ID NO: 711 - protein, 15 aa, Artificial Sequence
  HBV Pol_86
LSLDVSAAFYHIPLH

SEQ ID NO: 712 - protein, 15 aa, Artificial Sequence
  HBV Pol_87
SAAFYHIPLHPAAMP

SEQ ID NO: 713 - protein, 15 aa, Artificial Sequence
  HBV Pol_88
HIPLHPAAMPHLLVG

SEQ ID NO: 714 - protein, 15 aa, Artificial Sequence
  HBV Pol_89
PAAMPHLLVGSSGLP

SEQ ID NO: 715 - protein, 15 aa, Artificial Sequence
  HBV Pol_90
HLLVGSSGLPRYVAR

SEQ ID NO: 716 - protein, 15 aa, Artificial Sequence
  HBV Pol_91
SSGLPRYVARLSSTS

SEQ ID NO: 717 - protein, 15 aa, Artificial Sequence
  HBV Pol_92
RYVARLSSTSRNINY

SEQ ID NO: 718 - protein, 15 aa, Artificial Sequence
  HBV Pol_93
LSSTSRNINYQHGTM

SEQ ID NO: 719 - protein, 15 aa, Artificial Sequence
  HBV Pol_94
RNINYQHGTMQDLHD

SEQ ID NO: 720 - protein, 15 aa, Artificial Sequence
  HBV Pol_95
QHGTMQDLHDTCSRN

SEQ ID NO: 721 - protein, 15 aa, Artificial Sequence
  HBV Pol_96
QDLHDTCSRNLYVSL

SEQ ID NO: 722 - protein, 15 aa, Artificial Sequence
  HBV Pol_97
TCSRNLYVSLLLLYT

SEQ ID NO: 723 - protein, 15 aa, Artificial Sequence
  HBV Pol_98
LYVSLLLLYTTFGRK

SEQ ID NO: 724 - protein, 15 aa, Artificial Sequence
  HBV Pol_99
LLLYTTFGRKLHLYS

SEQ ID NO: 725 - protein, 15 aa, Artificial Sequence
  HBV Pol_100
TFGRKLHLYSHPIIL

SEQ ID NO: 726 - protein, 15 aa, Artificial Sequence
  HBV Pol_101
LHLYSHPIILGFRKI

SEQ ID NO: 727 - protein, 15 aa, Artificial Sequence
  HBV Pol_102
HPIILGFRKIPMGVG

SEQ ID NO: 728 - protein, 15 aa, Artificial Sequence
  HBV Pol_103
GFRKIPMGVGLSPFL

SEQ ID NO: 729 - protein, 15 aa, Artificial Sequence
  HBV Pol_104
PMGVGLSPFLLAQFT

SEQ ID NO: 730 - protein, 15 aa, Artificial Sequence
  HBV Pol_105
LSPFLLAQFTSAICS

SEQ ID NO: 731 - protein, 15 aa, Artificial Sequence
  HBV Pol_106
LAQFTSAICSVVRRA

SEQ ID NO: 732 - protein, 15 aa, Artificial Sequence
  HBV Pol_107
SAICSVVRRAFPHCL

SEQ ID NO: 733 - protein, 15 aa, Artificial Sequence
  HBV Pol_108
VVRRAFPHCLAFSYM

SEQ ID NO: 734 - protein, 15 aa, Artificial Sequence
  HBV Pol_109
FPHCLAFSYMDDVVL

SEQ ID NO: 735 - protein, 15 aa, Artificial Sequence
  HBV Pol_110
AFSYMDDVVLGAKSV

SEQ ID NO: 736 - protein, 15 aa, Artificial Sequence
  HBV Pol_111
DDVVLGAKSVQHLES

SEQ ID NO: 737 - protein, 15 aa, Artificial Sequence
  HBV Pol_112
GAKSVQHLESLFTAV

SEQ ID NO: 738 - protein, 15 aa, Artificial Sequence
  HBV Pol_113
QHLESLFTAVTNFLL

SEQ ID NO: 739 - protein, 15 aa, Artificial Sequence
  HBV Pol_114
LFTAVTNFLLSLGVH

SEQ ID NO: 740 - protein, 15 aa, Artificial Sequence
  HBV Pol_115
TNFLLSLGVHLNPTK

SEQ ID NO: 741 - protein, 15 aa, Artificial Sequence
  HBV Pol_116
SLGVHLNPTKTKRWG

SEQ ID NO: 742 - protein, 15 aa, Artificial Sequence
  HBV Pol_117
LNPTKTKRWGYSLNF

SEQ ID NO: 743 - protein, 15 aa, Artificial Sequence
  HBV Pol_118
TKRWGYSLNFMGYVI

SEQ ID NO: 744 - protein, 15 aa, Artificial Sequence
  HBV Pol_119
YSLNFMGYVIGSWGT

SEQ ID NO: 745 - protein, 15 aa, Artificial Sequence
  HBV Pol_120
MGYVIGSWGTLPQEH

SEQ ID NO: 746 - protein, 15 aa, Artificial Sequence
  HBV Pol_121
GSWGTLPQEHIVHKI

SEQ ID NO: 747 - protein, 15 aa, Artificial Sequence
  HBV Pol_122
LPQEHIVHKIKQCFR

SEQ ID NO: 748 - protein, 15 aa, Artificial Sequence
  HBV Pol_123
IVHKIKQCFRKLPLN

SEQ ID NO: 749 - protein, 15 aa, Artificial Sequence
  HBV Pol_124
KQCFRKLPLNRPIDW

SEQ ID NO: 750 - protein, 15 aa, Artificial Sequence
  HBV Pol_125
KLPLNRPIDWKVCQR

SEQ ID NO: 751 - protein, 15 aa, Artificial Sequence
  HBV Pol_126
RPIDWKVCQRIVGLL

SEQ ID NO: 752 - protein, 15 aa, Artificial Sequence
  HBV Pol_127
KVCQRIVGLLGFAAP

SEQ ID NO: 753 - protein, 15 aa, Artificial Sequence
  HBV Pol_128
IVGLLGFAAPFTQCG

SEQ ID NO: 754 - protein, 15 aa, Artificial Sequence
  HBV Pol_129
GFAAPFTQCGYPALM

SEQ ID NO: 755 - protein, 15 aa, Artificial Sequence
  HBV Pol_130
FTQCGYPALMPLSAC

SEQ ID NO: 756 - protein, 15 aa, Artificial Sequence
  HBV Pol_131
YPALMPLSACIQAKR

SEQ ID NO: 757 - protein, 15 aa, Artificial Sequence
  HBV Pol_132
PLSACIQAKRAFTFS

SEQ ID NO: 758 - protein, 15 aa, Artificial Sequence
  HBV Pol_133
IQAKRAFTFSPTYRA

SEQ ID NO: 759 - protein, 15 aa, Artificial Sequence
  HBV Pol_134
AFTFSPTYRAFLCKQ

SEQ ID NO: 760 - protein, 15 aa, Artificial Sequence
  HBV Pol_135
PTYRAFLCKQYMNLY

SEQ ID NO: 761 - protein, 15 aa, Artificial Sequence
  HBV Pol_136
FLCKQYMNLYPVARQ

SEQ ID NO: 762 - protein, 15 aa, Artificial Sequence
  HBV Pol_137
YMNLYPVARQRPGLC

SEQ ID NO: 763 - protein, 15 aa, Artificial Sequence
  HBV Pol_138
PVARQRPGLCQVFAD

SEQ ID NO: 764 - protein, 15 aa, Artificial Sequence
  HBV Pol_139
RPGLCQVFADATPTG

SEQ ID NO: 765 - protein, 15 aa, Artificial Sequence
  HBV Pol_140
QVFADATPTGWGLAI

SEQ ID NO: 766 - protein, 15 aa, Artificial Sequence
  HBV Pol_141
ATPTGWGLAIGHQRM

SEQ ID NO: 767 - protein, 15 aa, Artificial Sequence
  HBV Pol_142
WGLAIGHQRMRGTFV

SEQ ID NO: 768 - protein, 15 aa, Artificial Sequence
  HBV Pol_143
GHQRMRGTFVAPLPI

SEQ ID NO: 769 - protein, 15 aa, Artificial Sequence
  HBV Pol_144
RGTFVAPLPIHTAEL

SEQ ID NO: 770 - protein, 15 aa, Artificial Sequence
  HBV Pol_145
APLPIHTAELLAACF

SEQ ID NO: 771 - protein, 15 aa, Artificial Sequence
  HBV Pol_146
HTAELLAACFARSRS

SEQ ID NO: 772 - protein, 15 aa, Artificial Sequence
  HBV Pol_147
LAACFARSRSGAKLI

SEQ ID NO: 773 - protein, 15 aa, Artificial Sequence
  HBV Pol_148
ARSRSGAKLIGTDNS

SEQ ID NO: 774 - protein, 15 aa, Artificial Sequence
  HBV Pol_149
GAKLIGTDNSVVLSR

SEQ ID NO: 775 - protein, 15 aa, Artificial Sequence
  HBV Pol_150
GTDNSVVLSRKYTSF

SEQ ID NO: 776 - protein, 15 aa, Artificial Sequence
  HBV Pol_151
VVLSRKYTSFPWLLG

SEQ ID NO: 777 - protein, 15 aa, Artificial Sequence
  HBV Pol_152
KYTSFPWLLGCAANW

SEQ ID NO: 778 - protein, 15 aa, Artificial Sequence
  HBV Pol_153
PWLLGCAANWILRGT

SEQ ID NO: 779 - protein, 15 aa, Artificial Sequence
  HBV Pol_154
CAANWILRGTSFVYV

SEQ ID NO: 780 - protein, 15 aa, Artificial Sequence
  HBV Pol_155
ILRGTSFVYVPSALN

SEQ ID NO: 781 - protein, 15 aa, Artificial Sequence
  HBV Pol_156
SFVYVPSALNPADDP

SEQ ID NO: 782 - protein, 15 aa, Artificial Sequence
  HBV Pol_157
PSALNPADDPSRGRL

SEQ ID NO: 783 - protein, 15 aa, Artificial Sequence
  HBV Pol_158
PADDPSRGRLGLYRP

SEQ ID NO: 784 - protein, 15 aa, Artificial Sequence
  HBV Pol_159
SRGRLGLYRPLLRLP

SEQ ID NO: 785 - protein, 15 aa, Artificial Sequence
  HBV Pol_160
GLYRPLLRLPFRPTT

SEQ ID NO: 786 - protein, 15 aa, Artificial Sequence
  HBV Pol_161
LLRLPFRPTTGRTSL

SEQ ID NO: 787 - protein, 15 aa, Artificial Sequence
  HBV Pol_162
FRPTTGRTSLYAVSP

SEQ ID NO: 788 - protein, 15 aa, Artificial Sequence
  HBV Pol_163
GRTSLYAVSPSVPSH

SEQ ID NO: 789 - protein, 15 aa, Artificial Sequence
  HBV Pol_164
YAVSPSVPSHLPDRV

SEQ ID NO: 790 - protein, 15 aa, Artificial Sequence
  HBV Pol_165
SVPSHLPDRVHFASP

SEQ ID NO: 791 - protein, 15 aa, Artificial Sequence
  HBV Pol_166
LPDRVHFASPLHVAW

SEQ ID NO: 792 - protein, 13 aa, Artificial Sequence
  HBV Pol_167
HFASPLHVAWRPP

SEQ ID NO: 793 - protein, 222 aa, Artificial Sequence
  SARS-CoV-2 M protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 794 - protein, 419 aa, Artificial Sequence
  SARS-CoV-2 NP protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 795 - protein, 1273 aa, Artificial Sequence
  SARS-CoV-2 S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 796 - protein, 843 aa, Artificial Sequence
  HBV Pol protein
MPLSYQHFRKLLLLDDEAGPLEEELPRLADEGLNHRVAEDLNLGDLNVSIPWTHKVGNFTGLYSSTVPVFNPEWKTPSFPNIHLKEDIINRCQQYVGPLTVNEKRRLKVTMPARFYPNLTKYLPLDKGIKPYYPEHIVNHYFQTRHYLHTLWKAGILYKRETTRSASFCGSPYSWEQELQHGTLVFQTSTRHGDESFGSQSSGILSRSPVGPGIRSQFKQSRLGLQPQQGSMASGKPGRSGIIRARVHPTTRQSFGVEPAGSGHIDNSTSSASSCLHQSAVRKTAYSHLSTSKRQSSSGHAVELQHIPPSSARSQSEGPIPSCWWLQFRNSKPCSDYCLSHIVNLLEDWGPCTEYGEHHIRIPRTPARVTGGVFLVDKNPHNTTESRLVVDFSQFSRGSTHVFWPKFAVPNLQSLTNLLSSNLSWLSLDVSAAFYHIPLHPAAMPHLLVGSSGLPRYVARLSSTSRNINYQHGTMQDLHDTCSRNLYVSLLLLYTTFGRKLHLYSHPIILGFRKIPMGVGLSPFLLAQFTSAICSVVRRAFPHCLAFSYMDDVVLGAKSVQHLESLFTAVTNFLLSLGVHLNPTKTKRWGYSLNFMGYVIGSWGTLPQEHIVHKIKQCFRKLPLNRPIDWKVCQRIVGLLGFAAPFTQCGYPALMPLSACIQAKRAFTFSPTYRAFLCKQYMNLYPVARQRPGLCQVFADATPTGWGLAIGHQRMRGTFVAPLPIHTAELLAACFARSRSGAKLIGTDNSVVLSRKYTSFPWLLGCAANWILRGTSFVYVPSALNPADDPSRGRLGLYRPLLRLPFRPTTGRTSLYAVSPSVPSHLPDRVHFASPLHVAWRPP

SEQ ID NO: 797 - protein, 389 aa, Artificial Sequence
  HBV E protein
MGTNLSVPNPLGFFPDHQLDPAFGANSNNPDWDFNPNKDQWPAANQVGVGSFGPGFTPPHGNLLGWSPQAQGILTTVPAAPPPASTNRQSGRQPTPISLPLRDSHPQAMQWNSSTFHQALLDPKVRGLYLPAGGSSSGTVNPVQTTASPISSIFSRTGDPAPNMESTTSGFLGPLLVLQAGFFLLTRILTIPQSLDSWWTSLNFLGGAPTCSGQNLQSPTSNHSPTSCPPICPGYRWMCLRRFIIFLFILLLCLIFLLVLLDYQGMLPVCPLLPGTSTTSMGPCKTCTTPAQGTSMFPSCCCTQPSDGNCTCIPIPSSWAFARFLWEWASVRFSWLSLLVPFVQWFVGLSPTVWLSVIWMMWYWGRSLYNILNPFLPLLPIFFCLWVYI

SEQ ID NO: 798 - protein, 212 aa, Artificial Sequence
  HBV C protein
MQLFHLCLIISCLRPTVQASKLCLGWLWGMDIDPYKEFGASVELLSFLPSDFFPNIRDLLDTASALFREALESPEHCTPHHTAIRQAILCWGELMNLATWVGSNLEDPASRELVVGYVNVNMGLKIRQLLWFHISCLTFGRETVLEYLVSFGVWIRTPPAYRPPNAPILSTLPETTVVRRRGRSPRRRTPSPRRRRSQSPRRRRSQSRGSQC

SEQ ID NO: 799 - protein, 154 aa, Artificial Sequence
  HBV X protein
MAARLCCQLDPARDVLCLRPVGAESRGRPVSGAFGTLPSPSSSAVPTDHGAHLSLRGLPVCAFSSAGPCALRFTSARRMETTVNARQVLPKVLHKRTLGLSAMSTTDLEAYFKDRVFKDWEELGEETRLMIFVLGGCRHKLVCSPAPCNFFTSA

(19) United States
(12) Patent Application Publication
(10) Pub. No.: US 2023/0393121 A1
(43) Pub. Date: Dec. 7, 2023

(54) A METHOD TO MONITOR VIRUS-SPECIFIC T CELLS IN BIOLOGICAL SAMPLES

(71) Applicant: National University of Singapore, Singapore (SG)

(72) Inventors: Antonio Bertoletti, Singapore (SG); Anthony Tanoto Tan, Singapore (SG); Nina Le Bert, Singapore (SG)

(73) Assignee: National University of Singapore, Singapore (SG)

(21) Appl. No.: 18/033,032

(22) PCT Filed: Oct. 18, 2021
(86) PCT No.: PCT/SG2021/050627

§ 371 Date: Apr. 20, 2023

(30) Foreign Application Priority Data
Oct. 20, 2020 (SG) 10202010411P

(51) Int. Cl. G01N 33/50

(52) U.S. Cl. CPC G01N 33/5091; G01N 33/5023; G01N 2333/165; G01N 2333/02; G01N 33/505

(57) ABSTRACT

The present invention relates to a method of diagnosing and/or monitoring of virus infection and/or response to vaccination by generating a profile of a virus-specific T cell response that can (i) discriminate between virus infected and uninfected individuals, (ii) determine the effect of vaccination on T cell response, and (iii) determine the effect of viral variants on T cell response. More particularly, the described virus-specific T cell profiling is based on the detection of activated antigen-specific T lymphocytes responding to pools of selected short peptides from virus proteins. These peptide sequences have been selected for their immunogenicity. The profiling is typically performed using ELISPOT, but may also be performed using other techniques such as qPCR, more particularly direct qPCR. The present invention also includes kits for use in the methods of the invention.

### FIELD OF THE INVENTION

The present invention relates to a method of diagnosing and/or monitoring of virus infection and/or response to vaccination by generating a profile of a virus-specific T cell response that can (i) discriminate between virus infected and uninfected individuals, (ii) determine the effect of vaccination on T cell response, and (iii) determine the effect of viral variants on T cell response. More particularly, the described virus-specific T cell profiling is based on the detection of activated antigen-specific T lymphocytes responding to pools of selected short peptides from virus proteins. These peptide sequences have been selected for their immunogenicity. The profiling is typically performed using ELISPOT, but may also be performed using other techniques such as qPCR, more particularly direct qPCR. The present invention also includes kits for use in the methods of the invention.

### BACKGROUND OF THE INVENTION

The control and the long-term protection against viral infections require the coordinated activation of humoral (antibodies) and cellular (T cells) immunity. Viruses are intracellular pathogens, and CD8 T cells are necessary to recognize and lyse the infected cells. In addition, CD4 helper T cells are necessary to boost the maturation of antibody production.

Despite this important role in antiviral immunity, quantification of virus-specific T cells is technically complex in comparison to methods of antibody detection. As such virus-specific T cells are not routinely measured as a potential correlate of protection.

This is particularly problematic in the present COVID-19 pandemic, since measuring SARS-CoV-2-specific antibodies might not be sufficient to fully gauge the level of antiviral immunity induced by the infection.

The inventors and others have recently shown that SARS-CoV-2-specific T cells are present in 100% of COVID-19 convalescents (Grifoni A., et al., Cell Host Microbe 27(4): 671-680 (2020); Braun J., et al., 2020 medRxiv 1-12. doi:10.1101/2020.04.17.20061440; Dong T., et al., 2020 bioRxiv 1-36. doi:10.1101/2020.06.05.134551; Le Bert N., et al., Nature 2020; 584(7821): 457-62), while observations of SARS-CoV-2 infection with fading antibody titers have also been reported (Long Q.-X., et al., 2020 Nat Med 1-15. doi:10.1038/s41591-020-0897-1). A discrepancy between virus-specific T cell and antibody response was also reported in infections with the Middle East Respiratory Syndrome (MERS), where a quarter of MERS-infected patients showed only MERS-specific T cells (Zhao J., et al., 2017 Science Immunology 2, eaan5393. doi:10.1126/sciimmunol.aan5393).

However, quantification of SARS-CoV-2-specific T cells and, indeed, other virus-specific T cells in the general population is rarely performed since the methods of detection are complex and cumbersome and require very specific technical personnel and assays that require an initial step of separation of peripheral blood mononuclear cells (PBMC) from whole blood and experimental procedures (ELISPOT or Intracellular cytokine staining or qPCR) that necessitate specialized skills and equipment.

There is a need for a test that indicates a patient's level of virus-specific cellular immunity.

### SUMMARY OF THE INVENTION

The present invention provides methods to quantify virus-specific T cell activation, which have several applications.
1. A method of discriminating past or currently virus-infected subjects from virus un-infected subjects;
2. A method of testing T cell responses in vaccinated subjects; and
3. A method of testing whether T cells previously exposed to a virus or vaccine are activated by a virus variant, particularly a variant of concern (VOC).

SARS-CoV-2 and HBV are exemplified herein.

In a first aspect the invention provides an in vitro method of discriminating past or currently virus-infected subjects from virus un-infected subjects, comprising:
assaying a sample comprising or derived from blood, broncholavage (BAL fluid), nasal swabs, or nasopharyngeal aspirate from a subject to determine whether it comprises T cells reactive to one or more virus peptide pools, wherein said peptide pools are separately derived from virus antigenic structural and/or non-structural proteins, wherein;
(a) if the sample T cells are reactive to a majority of the peptide pools derived from the virus antigenic proteins, in comparison to unstimulated or DMSO treated cells, the subject is identified as past or currently infected by the virus, or
(b) if the sample T cells are reactive to 0, or a minority of the peptide pools derived from the virus antigenic proteins, in comparison to unstimulated or DMSO treated cells, the subject is identified as having been uninfected by the virus.

In some embodiments the virus is an enveloped virus. The antigenic structural and non-structural proteins of these viruses are well-known. An example is the membrane (M), nucleoprotein (NP) and/or Spike (S) proteins of an enveloped virus such as a coronavirus. Other enveloped viruses include Hepatitis B virus (HBV), wherein the antigenic proteins are Polymerase (Pol), Envelope (E), Core (C) and X.

In some embodiments of any aspect of the invention, the virus is a coronavirus, such as SARS, MERS or SARS-CoV-2.

In some embodiments, the virus is SARS-CoV-2 or HBV or variant thereof.

In a second aspect the invention provides an in vitro method of determining whether a vaccinee or previously virus-infected subject has T cells whose activation may be reduced by a virus variant, such as a variant of concern (VOC), comprising:
assaying a sample comprising or derived from blood, bronchoalveolar lavage (BAL fluid), nasal swabs, or nasopharyngeal aspirate from a subject to determine whether it comprises T cells reactive to one or more virus peptide pools, wherein said peptide pools are separately derived from (A) the whole virus antigenic protein present in the vaccine or corresponding to an antigenic protein from the virus that infected the subject, (B) non-conserved regions of said virus antigenic protein that are mutated in the virus variant, and (C) virus variant mutated non-conserved regions of the vaccine antigenic protein or corresponding to an antigenic protein from the virus that infected the subject, wherein;
the number or proportion of reactive T cells present in each pool is analyzed and utilized to derive in each single individual, the frequency of T cells directed towards the whole virus antigenic protein (PBMC stimulated with peptide pool A), the frequency of T cells directed toward non-conserved regions of said virus antigenic protein that are mutated in the virus variant (PBMC stimulated with Pool B) and the frequency of T cells inhibited by amino acid mutations present in virus variant mutated non-conserved regions (PBMC stimulated with pool C), wherein;
(a) if the sample T cells are reactive to peptide pool A, the subject has T cells responsive against the virus antigenic protein, and
(b) if the sample T cells are similarly reactive to pool B and pool C, the impact of the amino acid mutations in the variant are negligible on the total T cell response against the said virus antigenic protein;
(c) if the sample T cells react differently to pool B and pool C, the impact of the amino acid mutations in the variant on the total T cell response against the said virus antigenic protein can be estimated by the proportion of pool C against pool B response,
wherein the method provides an estimation of the ability of T cells of the subject to recognize the conserved and non-conserved region of different vaccine antigenic proteins or virus that infected the subject, and of the ability of mutations to reduce the T cell response towards variants.

In a third aspect the invention provides a method to quantify the presence of virus-specific T cells in a biological sample comprising or derived from blood, bronchoalveolar lavage (BAL fluid), nasal swabs, or nasopharyngeal aspirate from a subject, comprising;
a) Mixing the biological sample with one or more virus peptide pools, wherein said peptide pools are separately derived from virus antigenic structural and/or non-structural proteins;
b) Incubating the mixture formed for a period to allow T cell activation;
c) Rupture the cells from b);
d) Aliquot a sample from c) into PCR reagents, ACTIN (or other internal control) forward and reverse primers, ACTIN (or other internal control) probe, CXCL10 forward and reverse primers and CXCL10 probe for dqPCR; and/or
e) Extract RNA from a sample from c) and add a portion into PCR reagents, ACTIN (or other internal control) forward and reverse primers, ACTIN (or other internal control) probe, CXCL10 forward and reverse primers and CXCL10 probe for qPCR;
f) Perform cycles of dqPCR and/or qPCR for d) and e), respectively; and
g) Quantitate the expression of CXCL10 in the sample and compare with a control,
wherein an elevated CXCL10 level indicates the presence of virus-specific T cells in the subject sample.

It would be understood that whether a single peptide pool is sufficient for an assay method may depend on whether a subject has only been exposed to a part of the virus (e.g. spike protein vaccine) or a whole virus (virus-infected). Using a peptide pool specific against a single viral protein only informs the response against that particular protein. Therefore, to know if an individual was infected before using the PCR method, one would still need to assess the response against multiple proteins. This logic does not change with different readouts like PCR, ELISPOT or cytokine release assay.

In a fourth aspect the invention provides a method of treatment comprising administering, to a subject with T cells reactive to a majority of peptide pools derived from virus antigenic proteins, an effective amount of a virus inhibitor.

In some embodiments of the method of treatment, the peptide pools are selected from:
i) one or more M, NP and S pools for a coronavirus, or
ii) one or more C, Pol, X, and E pools for HBV.

In some embodiments of the method of treatment, the peptide pools are selected from:
i) one or more M, NP and S pools listed in Tables 1-4 for SARS-CoV-2, or ii) one or more C, Pol, X, and E pools listed in Tables 20-27 for HBV.

In a fifth aspect the invention provides a method of prophylaxis comprising administering, to a subject with T cells reactive to a minority of peptide pools derived from virus antigenic proteins, an effective amount of a virus vaccine.

In a sixth aspect the invention provides a method of monitoring the efficacy of a virus vaccine, comprising testing whether the recipient of said vaccine has T cells reactive to a minority or majority of peptide pools derived from said virus antigenic proteins.

In a seventh aspect the invention provides a kit to discriminate past or currently virus-infected subjects from virus un-infected subjects, the kit comprising a plurality of virus antigenic peptides that stimulate virus-exposed T cells, wherein the virus antigenic peptides are in peptide pools derived from:
I) M, NP and/or S proteins or
ii) C, Pol, X and/or E proteins.

In some embodiments,
i) the M protein comprises the amino acid sequence set forth in SEQ ID NO: 793;
ii) the NP protein comprises the amino acid sequence set forth in SEQ ID NO: 794;
iii) the S protein comprises the amino acid sequence set forth in SEQ ID NO: 795;
iv) the C protein comprises the amino acid sequence set forth in SEQ ID NO: 798;
v) the E protein comprises the amino acid sequence set forth in SEQ ID NO: 797;
vi) the X protein comprises the amino acid sequence set forth in SEQ ID NO: 799;
vii) the Pol protein comprises the amino acid sequence set forth in SEQ ID NO: 796.

In some embodiments the kit can quantify virus-specific T cell activation in an isolated patient sample, comprising one or more peptide pools, wherein said peptide pools are separately derived from virus antigenic proteins, such as membrane (M), nucleoprotein (NP) and/or Spike (S) proteins; or Core (C), Polymerase (Pol), X and/or Envelope (E) proteins.

In some embodiments, the kit further comprises:
i) PCR reagents; and/or
ii) primers and probes to detect CXCL10 and/or IFN-gamma expression by stimulated T cells.

In an eighth aspect the invention provides a set of 2 to 4 separate pools of peptides suitable to discriminate;
a) past or currently SARS-CoV-2-infected subjects from SARS-CoV-2 un-infected subjects;
b) past or currently HBV-infected subjects from HBV un-infected subjects,
   wherein the peptide pools are selected from those listed in Tables 1 to 4 and 8-19 for (a) and Tables 20-27 for (b).

In a ninth aspect the invention provides use of a kit of any one of aspects 7 to 9 in a method according to any one of aspects 1 to 6.

### BRIEF DESCRIPTION OF THE FIGURES

FIG. 1 shows cytokine secretion by T cells reactive to different pools of Spike peptides in COVID-19 convalescents. Spike is a long protein with 1276 amino acids, thus it requires 253 15-mer peptides overlapping by 10 amino acids to cover the whole protein, thus 7 pools of about 40 peptides. To reduce the number of peptides pools to test, we selected a single “Spike pool” comprised of 55 peptides. The 55 peptides cover 40.5% of the Spike protein. The frequency of reactive cells to the selected Spike pool (right) was compared to the 7 pools of 15-mers overlapping by 10 amino acids covering together the entire Spike protein (S1-S7) in 15 COVID-19 convalescents.

FIG. 2 shows a schematic representation of both approaches to profile SARS-CoV-2 specific T cells.

FIG. 3 shows (A) ELISPOT assay of PBMCs with SARS-CoV-2 M, NP1, NP2 and spike peptide pools can discriminate between infected and unexposed individuals; (B) Infected individuals are almost always positive for 3 or more peptide pools, while unexposed individuals occasionally have responses to 1-2 peptide pools. Grey shaded areas denote the threshold of positivity.

FIG. 4 shows (A) The concentration of IFN-γ in the plasma collected from whole blood of uninfected (n=9) and infected (n=6) individuals stimulated with the respective peptide pools was quantified. SARS-CoV-2 specific T cell response profile evaluated using this method can also discriminate between infected and uninfected individuals; (B) Infected individuals are positive for all peptide pools, while unexposed individuals occasionally have responses to 1-2 peptide pools. Grey shaded areas denote the threshold of positivity.

FIG. 5 shows (A) a schematic of how T cell responses against variants of concern (VOC) can be evaluated using the SARS-CoV-2 delta variant as an example. Vertical bar regions refer to amino acid mutations present in the delta variant compared to the wild-type SARS-CoV-2. Pool A contains peptides covering the whole Spike-Wuhan protein. Pool B contains peptides covering the non-conserved Spike-Wuhan regions affected by mutations present in the SARS-CoV-2 delta variant (B.1.617.2). Pool C contains peptides with the delta variant (B.1.617.2) amino acid mutations present in the non-conserved Spike-Wuhan regions. (B) Wells show the ELISPOT results from a vaccinee tested using the peptide pools described in (A). This vaccinee has a strong T cell response against the Spike protein as expected (Pool A, 133 spots/400,000 PBMC) and negligible T cell responses directed towards regions mutated in the delta variant (Pool B, 6 spots/400,000 PBMC). Since responses against these regions are very low, the impact of the amino acid mutations in the delta variant are negligible (Pool C VS Pool B). 2×Negative control wells are shown on the left.

FIG. 6 shows (A). Schematic of workflow for the three T cells activation (TACT) assays described. All assays begin with whole blood collection followed by overnight stimulation with nucleocapsid (NP) or spike (S) peptide pools. For TACTseq (FIG. 6), RNA was extracted and used for NGS using the Illumina system. For qTACT (FIG. 2), RNA was extracted and probe-based qPCR was performed using the BioRad CFX96/384 or Hyris bCUBE 2.0. For dqTACT (FIG. 3), blood was diluted and used directly for qPCR using the Hyris bCUBE 2.0. (B). TACTseq assay. Number of differentially expressed genes stimulated in whole blood by each peptide pool versus DMSO, grouped by subject COVID status. Significantly differentially expressed genes were defined as having p-value<0.05 and log 2FC>1. P-values were calculated using DESeq2 and adjusted using the Benjamini-Hochberg method. (C). Candidate genes selected for downstream validation based on differential expression versus DMSO, grouped by subject COVID status. Comparisons show significance calculated using DESeq2 and corrected using the Benjamini-Hochberg method.

FIG. 7 shows candidate genes selected for downstream validation based on differential expression versus DMSO. Comparisons show significance calculated using DESeq2 and corrected using the Benjamini-Hochberg method.

FIG. 8 shows (A). qPCR validation of 3 target genes (CXCL10, IFNG, IL2) on 11 naïve and 8 COVID-19 convalescent individuals. Results are plotted as gene expression relative to ACTIN minus DMSO control. Samples were run on the Hyris bCUBE 2.0. Comparisons show significance for the Wilcoxon Rank Sum two-sided test, corrected using the Benjamini-Hochberg method (*p<=0.05, **p<=0.01, ***p<=0.001, ****p<=0.0001). (B). Correlation between independent CXCL10, IFNG and ANKRD22 qPCR runs using samples from naïve and COVID-19 convalescent individuals treated with the spike protein. Values are calculated by comparing the final result (gene expression relative to ACTIN minus DMSO control) between all combinations of runs. Samples were run on the BioRad CFX96 or CFX384.

FIG. 9 shows qTACT assay. (A). Normalized CXCL10 mRNA expression (relative to ACTIN minus DMSO) before, 10 days, and 20 days after the first and second vaccine doses in SARS-CoV-2 naïve (black) and previously infected (grey) individuals. The qTACT assay was completed as shown in FIG. 6A (middle). The samples were run on a BioRad CFX384. Comparisons show significance for the Wilcoxon Rank Sum two-sided test, corrected using the Benjamini-Hochberg method (*p<=0.05, **p<=0.01, ***p<=0.001, ****p<=0.0001). (B). Normalized IFNG mRNA expression (relative to ACTIN minus DMSO) before, 10 days, and 20 days after the first and second vaccine doses in SARS-CoV-2 naïve (black) and previously infected (grey) individuals. The qTACT assay was completed as shown in FIG. 6A (middle). The samples were run on the Hyris bCUBE 2.0. Comparisons show significance for the Wilcoxon Rank Sum two-sided test, corrected using the Benjamini-Hochberg method (*p<=0.05, **p<=0.01, ***p<=0.001, ****p<=0.0001).

FIG. 10 shows (A). Correlation between CXCL10 mRNA expression (determined by the qTACT assay) and IFN-γ protein secretion (determined by ELLA) for the cohort described in FIG. 9 (B). Correlation between IFNG mRNA expression (determined by the qTACT assay) and IFN-γ protein secretion (determined by ELLA) for the cohort described in FIG. 9.

FIG. 11 shows a dqTACT assay. (A). Image showing all reagents and equipment needed to perform the dqTACT assay. With appropriate biosafety level 2 requirements and a cell culture incubator, this is the minimum reagents and equipment required to run the dqTACT assay which include in clockwise order: the Hyris bCube 2.0 qPCR machine, pipettes and tips, a heparin coated blood collection tube, Hyris 16/32 well cartridges, nuclease-free water, Jena Bioscience SCRIPT direct RT-qPCR ProbesMaster mix, PCR primer/probes, and RPMI medium. (B). Normalized CXCL10 expression (peptides stimulated relative to ACTIN minus DMSO control) of naïve and COVID-19 vaccinated individuals (FIG. 9). The dqTACT assay was completed as shown in FIG. 6A (bottom). Comparisons show significance for the Wilcoxon Rank Sum two-sided test (*p<=0.05, **p<=0.01, ***p<=0.001, ****p<=0.0001). (C). Normalized CXCL10 secretion as quantified by ELLA (peptides stimulated minus DMSO control) of naïve and COVID-19 vaccinated individuals. Comparisons show significance for the Wilcoxon Rank Sum two-sided test (*p<=0.05, **p<=0.01, ***p<=0.001, ****p<=0.0001). (D). Normalized CXCL10 expression (peptides stimulated relative to ACTIN minus DMSO control) of subjects enrolled in the CombiVacS trial. All subjects received a first dose of ChAdOx1s from AstraZeneca (Dose 1 (AZ)). The patients were then divided into two groups and received either a second placebo dose (Dose 1 (AZ)+Dose 2 (placebo)) or a second dose of BNT162b2 from Pfizer (Dose 1 (AZ)+Dose 2 (Pfizer)). The dqTACT assay was completed as shown in FIG. 6A (bottom). Comparisons show significance for the Wilcoxon Rank Sum two-sided test (*p<=0.05, **p<=0.01, ***p<=0.001, ****p<=0.0001).

FIG. 12 (A-C) show results from IFN-γ, IL2, and TNFα ELLA showing normalized protein secretion (minus DMSO control) of naïve and COVID-19 vaccinated individuals. Comparisons show significance for the Wilcoxon Rank Sum two-sided test, corrected using the Benjamini-Hochberg method (*p<=0.05, **p<=0.01, ***p<=0.001, ****p<=0.0001). (D-G) show correlation between data shown in A-C and FIG. 11C (CXCL10 mRNA quantified by dqTACT vs. CXCL10, IFN-γ, IL2, and TNFα protein quantified by ELLA). Correlation coefficients and p-values were calculated using the Spearman method.

FIG. 13 shows the application of a whole blood cytokine release assay for the detection of HBV-specific T cells. A) Schematic showing the 4 proteins of Hepatitis B virus and the corresponding peptide pools. B) Whole blood cytokine release assay performed by stimulation of whole blood from a chronically infected HBV patient and a vaccinated individual with the corresponding peptide pool. IFN-γ and IL-2 secretion was measure by ELLA. The level of cytokines present in the plasma of DMSO controls was subtracted from the corresponding peptide pool stimulated samples.

### DEFINITIONS

Certain terms employed in the specification, examples and appended claims are collected here for convenience.

As used in the specification and the appended claims, the singular forms “a,” “an” and “the” include plural referents unless the context clearly dictates otherwise.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term ‘majority’ refers to a value over 50%. Conversely, the term ‘minority’ refers to a value under 50%. For example, if 3 or 4 of a total of 4 peptide pools positively activate T cells in a sample, compared to a control, a majority of pools are positive and the sample is indicative of the subject having been exposed to SARS-CoV-2 infection. When 4 pools M, NP1, NP2 and S were used, 50% or more (2, 3, or 4 pools out of 4) positive pools was considered to indicate the subject had been infected by SARS-CoV-2.

As used herein, the term ‘a subject uninfected by SARS-CoV-2’ refers to a subject who is considered to not have been exposed to and infected by SARS-CoV-2, for the purpose of the invention.

Any discussion of documents, acts, materials, devices, articles or the like which has been included in the present specification is not to be taken as an admission that any or all of these matters form part of the prior art base, or were common general knowledge in the field relevant to the present disclosure as it existed before the priority date of each of the appended claims.

Bibliographic references mentioned in the present specification are for convenience listed at the end of the examples. The whole content of such bibliographic references are herein incorporated by reference.

### DETAILED DESCRIPTION OF THE INVENTION

The present invention relates to a method of discriminating past or currently virus-infected subjects from virus un-infected subjects, based on the detection of activated antigen-specific T lymphocytes responding to selected peptide sequences from the virus in an isolated sample from said individual. These peptide sequences are selected for their immunogenicity and are represented in peptide pools separately derived from virus antigenic proteins, such as membrane (M), nucleoprotein (NP) and/or Spike (S) proteins; or Polymerase (Pol), Envelope (E), Core (C) and X proteins.

In addition, the present invention provides a method of testing T cell responses in vaccinated subjects. In this way, the efficacy of a vaccine to stimulate a T cell response can be determined.

Further, the invention provides a method of testing the impact of amino acid mutations in a virus strain on T cells that have previously been exposed to a parent or comparator strain. For example, different variants of concern (VOC) of SARS-CoV-2 have replaced world-wide the original SARS-CoV-2 Wuhan isolate. These VOCs are characterized by amino acid substitutions that provide biological advantages such as increased infectivity or escape humoral (Antibodies) but also cellular (T cells) immunity. We have designed a method based on specific combination of peptide pools covering both different SARS-CoV-2 proteins (i.e., Spike) and the corresponding regions affected by the amino acid mutations that are used to stimulate T cells, The results of this multiple stimulation strategy define with accuracy the impact of these amino acid mutations on the SARS-CoV-2-specific T cells induced by infection with Wuhan strain or by vaccination with the present vaccines based on the Wuhan strain. Presented herein as an example is a method to analyze the impact of amino acid mutations present in Spike in different VOCs (using Delta variant as an example) on the SPIKE specific T cells induced by vaccination or previous infection.

Discrimination can be achieved based on the number or proportion of peptide pools that stimulate the isolated T cells above a control value.

Each of the M, NP and S, or Pol, C, E and X, proteins may contribute at least one pool of immunogenic peptides. For example, the NP protein consists of 419 amino acids. It is possible that the NP protein could be divided into more than one pool comprising 15-mer peptides, such as 2 pools where 1 pool comprises 15-mer peptides which overlap adjacent peptides by 10 amino acids covering amino acids 1-215; and a second pool comprising 15-mers which overlap adjacent peptides by 10 amino acids covering amino acids 216-419. The peptide overlap can be seen, for example, in the NP peptides listed sequentially in Table 2. It would be understood that the degree of overlap between 15-mer peptides could be varied from 10 without substantially affecting the ability to activate T cells and obtain a valid result. Likewise, the S protein, which is 1273 amino acids in length, could be divided up into 1, 2, 3 or more pools of 15-mer peptides. The number and size of the pools needs to be balanced with practical considerations, such as the amount of blood sample available, the cost of generating peptide pools, and whether the number of pools improves discrimination.

In a first aspect the invention provides an in vitro method of discriminating past or currently virus-infected subjects from virus un-infected subjects, comprising:
assaying a sample comprising or derived from blood, broncholavage (BAL fluid), nasal swabs, or nasopharyngeal aspirate from a subject to determine whether it comprises T cells reactive to one or more virus peptide pools, wherein said peptide pools are separately derived from virus antigenic structural and non-structural proteins, wherein;
(a) if the sample T cells are reactive to a majority of the peptide pools derived from the virus antigenic proteins, in comparison to unstimulated or DMSO treated cells, the subject is identified as past or currently infected by the virus, or
(b) if the sample T cells are reactive to 0, or a minority of the peptide pools derived from the virus antigenic proteins, in comparison to unstimulated or DMSO treated cells, the subject is identified as having been uninfected by the virus.

In some embodiments the virus is an enveloped virus or a non-enveloped virus. The antigenic structural and non-structural proteins of these viruses are well-known. An example is the membrane (M), nucleoprotein (NP) and/or Spike (S) proteins of an enveloped viruses such as coronaviruses, whereas and Hepatitis B viruses (HBV) comprise polymerase (Pol), envelope (E), core (C) and X antigenic structural and non-structural proteins.

In some embodiments of any aspect of the invention, the virus is a coronavirus.

In some embodiments, the virus is a coronavirus, selected from the group comprising MERS-CoV, SARS-CoV, SARS-CoV-2, HKU1, OC43, NL63 and 229E or variant thereof.

In some embodiments, the virus is SARS-CoV-2 or variant thereof.

In some embodiments, the virus is HBV or variant thereof.

In some embodiments,
ai) an M peptide pool comprises or consists of at least one peptide derived from an M protein comprising the amino acid sequence set forth in SEQ ID NO: 793;
ii) an NP peptide pool comprises or consists of at least one peptide derived from an NP protein comprising the amino acid sequence set forth in SEQ ID NO: 794; and
iii) an S peptide pool comprises or consists of at least one peptide derived from an S protein comprising the amino acid sequence set forth in SEQ ID NO: 795; or
bi) a Pol peptide pool comprises or consists of at least one peptide derived from a Pol protein comprising the amino acid sequence set forth in SEQ ID NO: 796;
ii) an E peptide pool comprises or consists of at least one peptide derived from an E protein comprising the amino acid sequence set forth in SEQ ID NO: 797;
iii) a C peptide pool comprises or consists of at least one peptide derived from a C protein comprising the amino acid sequence set forth in SEQ ID NO: 798; and
iv) an X peptide pool comprises or consists of at least one peptide derived from an X protein comprising the amino acid sequence set forth in SEQ ID NO: 799.

In some embodiments;
ai) an M peptide pool comprises or consists of at least one peptide selected from peptides having the amino acid sequences set forth in SEQ ID Nos: 1-43,
ii) an NP peptide pool comprises or consists of at least one peptide selected from peptides having the amino acid sequences set forth in SEQ ID Nos: 44-125, and
iii) an S peptide pool comprises or consists of at least one peptide selected from peptides having the amino acid sequences set forth in SEQ ID Nos: 126-454.

In some embodiments, the NP peptide pool is divided into 2 pools, NP1 and NP2.

In some embodiments, the S peptide pool comprises or consists of at least one peptide selected from peptides having the amino acid sequences set forth in SEQ ID Nos: 126-180 (Table 4).

In some embodiments NP1 comprises or consists of at least one peptide selected from peptides having the amino acid sequences set forth in SEQ ID Nos: 44-84, and the NP2 peptide pool comprises or consists of at least one peptide selected from peptides having the amino acid sequences set forth in SEQ ID Nos: 85-125.

In some embodiments;
ai) the M peptide pool consists of peptides having the amino acid sequences set forth in SEQ ID Nos: 1-43,
ii) the NP1 peptide pool consists of peptides having the amino acid sequences set forth in SEQ ID Nos: 44-84,
iii) the NP2 peptide pool consists of peptides having the amino acid sequences set forth in SEQ ID Nos: 85-125, and
iv) the S peptide pool consists of peptides having the amino acid sequences set forth in SEQ ID Nos: 126-180.

In some embodiments;
bi) a Pol peptide pool comprises or consists of at least one peptide selected from peptides having the amino acid sequences set forth in SEQ ID Nos: 626-792 (Tables 24-27),
ii) an E peptide pool comprises or consists of at least one peptide selected from peptides having the amino acid sequences set forth in SEQ ID Nos: 550-625 (Tables 22-23);
iii) a C peptide pool comprises or consists of at least one peptide selected from peptides having the amino acid sequences set forth in SEQ ID Nos: 480-520 (Table 20); and
iv) an X peptide pool comprises or consists of at least one peptide selected from peptides having the amino acid sequences set forth in SEQ ID Nos: 521-549 (Table 21).

In some embodiments, the Pol peptide pool is divided into a plurality of pools, such as 2, 3 or 4 pools of peptides. Preferably, each of the plurality of Pol pools has approximately equal numbers of peptides. In some embodiments, the Pol peptide pool is divided into 4 pools, Pol-1, Pol-2, Pol-3 and Pol-4. An example is shown in Tables 24-27.

In some embodiments, the E peptide pool is divided into 2 pools, E-1 and E-2. An example is shown in Tables 22-23.

In some embodiments,
bi) the Pol peptide pool consists of peptides having the amino acid sequences set forth in SEQ ID Nos: 626-792;
ii) the E peptide pool consists of peptides having the amino acid sequences set forth in SEQ ID Nos: 550-625;
iii) the C peptide pool consists of peptides having the amino acid sequences set forth in SEQ ID Nos: 480-520; and
iv) the X peptide pool consists of peptides having the amino acid sequences set forth in SEQ ID Nos: 521-549.

In some embodiments;
(a) if the sample T cells are reactive to 3 or 4 of the peptide pools derived from M, NP1, NP2 and S, in comparison to unstimulated or DMSO treated cells, the subject is identified as past or currently infected by SARS-CoV-2, or
(b) if the sample T cells are reactive to 0, 1 or 2 of the peptide pools derived from M, NP1, NP2 and S, in comparison to unstimulated or DMSO treated cells, the subject is identified as having been uninfected by SARS-CoV-2.

In some embodiments, the method comprises the steps of:
a) mixing the sample with each of said peptide pools to produce:
i) assay samples corresponding to M, NP and S; or
ii) assay samples corresponding to E, Pol, C and X;
b) incubating each mixture for a period to allow for T cell activation;
c) measuring the level of at least one secreted cytokine in each said mixture and determining whether the level of at least one secreted cytokine is above a threshold control value to indicate a positive T-cell reaction; and
d) counting the number of peptide pools that are positive.

In some embodiments, the method comprises the steps of:
a) mixing the sample with each of said peptide pools to produce:
i) 4 assay samples corresponding to M, NP1, NP2 and S; or
ii) 8 assay samples corresponding to C, Pol1, Po12, Po13, Po14, E1, E2 and X;
b) incubating each mixture for a period to allow for T cell activation;
c) measuring the level of at least one secreted cytokine in each said mixture and determining whether the level of at least one secreted cytokine is above a threshold control value to indicate a positive T-cell reaction; and
d) counting the number of peptide pools that are positive.

In some embodiments, if the sample T cells are reactive to 50% or more of the peptide pools derived from SARS-CoV-2 M, NP and S, in comparison to unstimulated or DMSO-treated cells, the subject is identified as past or currently infected by virus.

In a second aspect the invention provides an in vitro method of determining whether a vaccinee or previously virus-infected subject has T cells whose activation may be reduced by a virus variant, such as a variant of concern (VOC), comprising:
assaying a sample comprising or derived from blood, bronchoalveolar lavage (BAL fluid), nasal swabs, or nasopharyngeal aspirate from a subject to determine whether it comprises T cells reactive to one or more virus peptide pools, wherein said peptide pools are separately derived from (A) the whole virus antigenic protein present in the vaccine or corresponding to an antigenic protein from the virus that infected the subject, (B) non-conserved regions of said virus antigenic protein that are mutated in the virus variant, and (C) virus variant mutated non-conserved regions of the vaccine antigenic protein or corresponding to an antigenic protein from the virus that infected the subject, wherein;
the number or proportion of reactive T cells present in each pool is analyzed and utilized to derive in each single individual, the frequency of T cells directed towards the whole virus antigenic protein (PBMC stimulated with peptide pool A), the frequency of T cells directed toward non-conserved regions of said virus antigenic protein that are mutated in the virus variant (PBMC stimulated with Pool B) and the frequency of T cells inhibited by amino acid mutations present in virus variant mutated non-conserved regions (PBMC stimulated with pool C), wherein;
(a) if the sample T cells are reactive to peptide pool A, the subject has T cells responsive against the virus antigenic protein, and
(b) if the sample T cells are similarly reactive to pool B and pool C, the impact of the amino acid mutations in the variant are negligible on the total T cell response against the said virus antigenic protein;
(c) if the sample T cells reacts differently to pool B and pool C, the impact of the amino acid mutations in the variant on the total T cell response against the said virus antigenic protein can be estimated by the proportion of pool C against pool B response,
   wherein the method provides an estimation of the ability of T cells of the subject to recognize the conserved and non-conserved region of different vaccine antigenic proteins or virus that infected the subject, and of the ability of mutations to reduce the T cell response towards variants.

In some embodiments, the virus is a coronavirus, selected from the group comprising MERS-CoV, SARS-CoV, SARS-CoV-2, KHU1, OC43, NL63 and 229E or variants thereof.

In some embodiments, the virus antigenic protein is an M, NP, or S protein.

In some embodiments, the virus is HBV and the virus antigenic protein is an E, Pol, C or X protein.

In some embodiments, peptide pool A and pool B are derived from a wild-type virus.

It would be understood that peptide pool A and pool B could be derived from a variant if it became a reference point due to becoming endemic or if future vaccines employ the variant sequence instead of the original wildtype virus.

In some embodiments of the method;
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C protein comprises the amino acid sequence set forth in SEQ ID NO: 798; and
iv) the X protein comprises the amino acid sequence set forth in SEQ ID NO: 799.

In some embodiments, the wildtype virus is SARS-CoV-2 wildtype and the variant is selected from the group comprising B.1.617.2 (Delta), B.1.1.7 (Alpha V1), B.1.351 (Beta V2), P.1 (Gamma, V3), B.1.617.1 (Kappa), P.2, B.1.427/9 (Epsilon), B.1.525 (Eta), B.1.526 (Iota), C.37 (Lambda), B.1.621 and B.1.620; or the virus is HBV C.

In some embodiments, the method comprises the steps of:
a) mixing the sample with each of said peptide pools A, B, and C to produce assay samples;
b) incubating each mixture formed for a period to allow T cell activation;
c) measuring the level of at least one secreted cytokine in each said mixture and determining whether the level of at least one secreted cytokine is above a threshold control value to indicate a positive T-cell reaction; and
d) determining the number or proportion of reactive T cells present in each pool.

In some embodiments, the secreted cytokine is selected from the group comprising IFN-gamma (IFN-γ), IL-2, CXCL9, CXCL10, TNF-alpha, IL-6, IL-10 and IL-1. Preferably IFN-gamma, IL-2, or CXCL10 levels are measured. Detection of the cytokine CXCL10 is preferred if qPCR is used to quantify T cell activation.

In some embodiments, the said cytokine level is determined by immunoassay, such as ELISA or ELISPOT, or by qPCR or direct qPCR.

According to the method, a sample is tested by separately mixing aliquots from the sample with a peptide pool representing at least a portion of M, NP or S protein, or with a peptide pool representing at least a portion of E, Pol, C or X protein to determine whether the sample comprises T cells reactive to M, NP and/or S peptides; or E, Pol, C and/or X peptides, respectively.

In some embodiments, the sample comprises whole blood, broncholavage (BAL fluid), nasal swabs, nasopharyngeal aspirate, or isolated peripheral blood mononuclear cells (PBMCs).

In some embodiments, when whole blood, broncholavage (BAL fluid), nasal swabs, or nasopharyngeal aspirate is used, the incubation period in step b) may be for between about 6 to 24 h.

In some embodiments of the method:
a) whole blood is mixed with each of said peptide pools;
b)i) each mixture is incubated for at least 6 h;
b)ii) a plasma fraction of the mixture is isolated;
c) the level of at least one secreted cytokine in each said plasma fraction is measured and compared to a threshold control value to indicate a positive or negative T cell reaction.

In some embodiments, the sample also comprises a concentration of DMSO and/or heparin. Heparin may be required particularly if whole blood is to be assayed, to inhibit coagulation.

In some embodiments, the control sample or threshold control value may be derived from an assay sample comprising a subject sample that is unstimulated or DMSO-treated.

In some embodiments of the method:
a) whole blood is mixed with heparin, DMSO and each of said peptide pools to produce:
i) 4 assay samples corresponding to M, NP1, NP2 and S; or
ii) 8 assay samples corresponding to E1, E2, Pol1, Po12, Po13, Po14, C and X;
b)i) each mixture is incubated overnight;
b)ii) a plasma fraction of the mixture is isolated;
c) the level of at least one secreted cytokine, selected from the group comprising IFN-gamma, IL-2, CXCL9, CXCL10, TNF-alpha, IL-6, IL-10 and IL-1, in each said plasma fraction is measured and compared to a threshold control value, derived from an assay sample comprising a subject sample that is unstimulated or DMSO-treated, to indicate a positive or negative T cell reaction.

In a third aspect the invention provides a method to quantify the presence of virus-specific T cells in a biological sample comprising or derived from blood, broncholavage (BAL fluid), nasal swabs, or nasopharyngeal aspirate from a subject, comprising;
a) Mixing the biological sample with one or more virus peptide pools, wherein said peptide pools are separately derived from virus antigenic structural or non-structural proteins;
b) incubating the mixture formed for a period to allow T cell activation;
c) Rupture the cells from b);
d) Aliquot a sample from c) into PCR reagents, ACTIN (or other internal control) forward and reverse primers, ACTIN (or other internal control) probe, CXCL10 forward and reverse primers and CXCL10 probe for dqPCR; and/or
e) extract RNA from a sample from c) and add a portion into PCR reagents, ACTIN (or other internal control) forward and reverse primers, ACTIN (or other internal control) probe, CXCL10 forward and reverse primers and CXCL10 probe for qPCR;
f) perform cycles of dqPCR and/or qPCR for d) and e), respectively; and
g) quantitate the expression of CXCL10 in the stimulated T cells and compare with a control,
wherein an elevated CXCL10 level indicates the presence of virus-specific T cells in the subject sample.

In some embodiments the virus is an enveloped virus. The antigenic structural and non-structural proteins of these viruses are well-known. An example is the membrane (M), nucleoprotein (NP) and/or Spike (S) proteins of a coronavirus. The antigenic proteins of Hepatitis B virus (HBV) are Pol, E, C and X proteins.

In some embodiments, the virus is a coronavirus, selected from the group comprising MERS-CoV, SARS-CoV, SARS-CoV-2, KHU1, OC43, NL63 and 229E or variant thereof; or a non-enveloped virus, such as HBV.

In some embodiments, the virus is SARS-CoV-2 or HBV.

In some embodiments:
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C protein comprises the amino acid sequence set forth in SEQ ID NO: 798; and
iv) the X protein comprises the amino acid sequence set forth in SEQ ID NO: 799.

In some embodiments, the peptide pools comprise one or more M, NP and S peptides listed in Tables 1-4 and 7-19; or one or more Pol, E, C and X peptides listed in Tables 20-27.

In a fourth aspect, the invention provides a method of treatment comprising administering, to a subject with T cells reactive to:
i) a majority of peptide pools derived from virus antigenic structural or non-structural proteins, an effective amount of a virus inhibitor; or
ii) 0, or a minority of the peptide pools M, NP and S; or E, Pol, C and X, an effective amount of a coronavirus or HBV vaccine, respectively.

In some embodiments, the virus is a coronavirus such as a coronavirus selected from the group comprising MERS-CoV, SARS-CoV, SARS-CoV-2, HKU1, OC43, NL63 and 229E or variants thereof.

In some embodiments, the virus is SARS-CoV-2.

In some embodiments the virus is HBV.

In some embodiments, the invention provides a method of treatment comprising administering, to a subject with T cells reactive to 3 or 4 of the peptide pools M, NP1, NP2 and S listed in Tables 1-4, an effective amount of a SARS-CoV-2 inhibitor.

In some embodiments, the invention provides a method of treatment comprising administering, to a subject with T cells reactive to 3 or 4 of the peptide pools M, NP1, NP2 and S listed in Tables 1-4, an effective amount of a SARS-CoV-2 inhibitor.

In some embodiments, the invention provides a method of treatment comprising administering, to a subject with T cells reactive to 5 to 8 of the pools E1, E2, Pol1, Pol2, Pol3, Pol4, C and X listed in Tables 20-27, an effective amount of a HBV inhibitor.

In a fifth aspect, the invention provides a method of prophylaxis comprising administering, to a subject with T cells reactive to 0, or a minority of peptide pools derived from virus antigenic structural and non-structural proteins, an effective amount of a virus vaccine.

In some embodiments the virus is an enveloped virus.

In some embodiments, the virus is a coronavirus such as a coronavirus selected from the group comprising MERS-CoV, SARS-CoV, SARS-CoV-2, HKU1, OC43, NL63 and 229E or variants thereof.

In some embodiments, the virus is SARS-CoV-2. In some embodiments the virus is HBV.

In some embodiments, the invention provides a method of prophylaxis comprising administering, to a subject with T cells reactive to 0, 1 or 2 of the peptide pools M, NP1, NP2 and S listed in Tables 1-4, an effective amount of a SARS-CoV-2 vaccine.

In some embodiments, the invention provides a method of prophylaxis comprising administering, to a subject with T cells reactive to 0, 1, 2, 3 or 4 of the peptide pools E1, E2, Pol1, Po12, Po13, Po14, C and X listed in Tables 20-27, an effective amount of a HBV vaccine.

In a sixth aspect the invention provides a method of monitoring the efficacy of a virus vaccine, comprising testing whether the recipient of said vaccine has T cells reactive to a minority, 50%, or majority of peptide pools derived from virus antigenic structural and non-structural proteins, such as virus M, NP and S proteins; or virus E, Pol, C and X proteins.

In some embodiments, the virus is a coronavirus such as a coronavirus selected from the group comprising MERS-CoV, SARS-CoV, SARS-CoV-2, HKU1, OC43, NL63 and 229E or variants thereof. In some embodiments, the virus is SARS-CoV-2.

In some embodiments the virus is HBV.

In some embodiments, the invention provides a method of monitoring the efficacy of a SARS-CoV-2 vaccine, comprising testing whether the recipient of said vaccine has T cells reactive to 0, 1, 2, 3 or 4 of the peptide pools M, NP1, NP2 and S listed in Tables 1-4 and 7-19.

In some embodiments, the invention provides a method of monitoring the efficacy of a HBV vaccine, comprising testing whether the recipient of said vaccine has T cells reactive to 0, 1, 2, 3, 4, 5, 6, 7 or 8 of the peptide pools E1, E2, Pol1, Pol2, Pol3, Pol4, C and X listed in Tables 20-27.

It would be understood that the pools used may depend on which of the virus antigenic proteins is/are used in the vaccine. The pool may be for a particular protein from a wildtype, or variant virus. Table 4 contains selected peptides of the spike protein that were tested and demonstrated to be good enough to estimate the total spike T cell response. This table of peptides do not cover the entire spike protein. This is used in conjunction with peptides from Tables 1-3 to detect if the subject is infected or not infected. Table 7 is the reference peptides that may be used to assess the T cell response against the delta VOC with the wildtype Wuhan as a reference. Tables 8-19 contain peptides derived from the VOC that are different from the Wuhan wildtype SARS-CoV-2 virus.

In a seventh aspect the invention provides a kit to discriminate past or currently virus-infected subjects from virus un-infected subjects, the kit comprising a plurality of virus structural or non-structural peptides that stimulate virus-exposed T cells, wherein the virus peptides are in peptide pools derived from virus antigenic structural or non-structural proteins.

In some embodiments the virus is an enveloped virus or a non-enveloped virus.

An example of the antigenic proteins is the membrane (M), nucleoprotein (NP) and/or Spike (S) proteins of an enveloped virus.

In some embodiments, the virus is a coronavirus such as a coronavirus selected from the group comprising MERS-CoV, SARS-CoV, SARS-CoV-2, HKU1, OC43, NL63 and 229E or variants thereof.

In some embodiments, the virus is SARS-CoV-2. In some embodiments the virus is HBV.

In some embodiments of the kit:
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C protein comprises the amino acid sequence set forth in SEQ ID NO: 798; and
iv) the X protein comprises the amino acid sequence set forth in SEQ ID NO: 799.

In some embodiments:
the M peptide pool comprises peptides having amino acid sequences set forth in SEQ ID Nos: 1-43;
the NP peptide pool comprises peptides having amino acid sequences set forth in SEQ ID Nos: 44-125;
the S peptide pool comprising peptides selected from peptides having amino acid sequences set forth in SEQ ID Nos: 126-454.

In some embodiments the kit further comprises one or more reagents to detect cytokines and/or chemokines secreted from activated T cells.

In some embodiments the kit further comprises:
i) PCR reagents and/or primers and probes to detect CXCL10 and/or IFN-gamma expression; and/or
ii) ELISPOT reagents.

In some embodiments, suitable primers and probes are as shown in Table 5.

In some embodiments, the kit comprises one or more peptide pools selected from the pools in Tables 7-19 rather than the pools in Tables 1-4. Such pools could be used to analyse the effect of virus variants, including variants of concern (VOC), on T cell activation in vaccinated or previously infected subjects.

In some embodiments the VOC are selected from the group comprising B.1.617.2 (Delta), B.1.1.7 (Alpha V1), B.1.351 (Beta V2), P.1 (Gamma, V3), B.1.617.1 (Kappa), P.2, B.1.427/9 (Epsilon), B.1.525 (Eta), B.1.526 (Iota), C.37 (Lambda), B.1.621 and B.1.620.

Herein is presented a method based on specific combination of peptide pools covering both different SARS-CoV-2 proteins (i.e., Spike) and the corresponding regions affected by the amino acid mutations that are used to stimulate T cells, The results of this multiple stimulation strategy define with accuracy the impact of these amino acid mutations on the SARS-CoV-2-specific T cells induced by infection with Wuhan strain or by vaccination with the present vaccines based on the Wuhan strain. Peptide pools directed to non-conserved regions of the Wuhan strain variants are shown in Tables 8 to 19, while the sequences of the regions of the Wuhan strain that correspond to the regions of the Delta variant (B.1.617.2) are shown in Table 7.

Presented herein is an example of a method to analyze the impact of AA mutations present in the Spike protein in different VOCs (using Delta variant as an example) on the SPIKE specific T cells induced by vaccination. It would be understood that pools derived from non-conserved regions of M or NP may be used to analyse the effect of VOCs, depending on the antigens the subject's T cells have been exposed to.

In an eighth aspect the invention provides a set of at least 2, at least 3, or at least 4 separate pools of peptides suitable to discriminate:
i) past or currently SARS-CoV-2-infected subjects from SARS-CoV-2 un-infected subjects, wherein the peptide pools are selected from those listed in Tables 1 to 4 and 7-19; or
ii) past or currently HBV-infected subjects from HBV un-infected subjects, wherein the peptide pools are selected from those listed in Tables 20-27.

In a ninth aspect the invention provides a use of a kit of aspect 7 in a method according to any one of aspects 1 to 6.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1

### SARS-CoV-2 Peptide Pools

An integral part of our invention is the selection of peptide pools necessary to define a profile of T cell responses that can differentiate individuals that have been primed by SARS-CoV-2 infection or individuals that were infected by other common cold coronaviruses. The SARS-CoV-2 specific T cell responses were profiled in individuals who were in contact with SARS-CoV-2 and tested antibody positive (Abbot test/anti-NP antibody) or were positive in a surrogate virus neutralization assay (sVNT) at the time of the T cell test (symptomatic n=35; asymptomatic n=73), and compared the profile to that obtained from healthy donors (n=51) without any history of SARS-CoV-2 contact and negative for anti-NP SARS-CoV-2 antibodies.

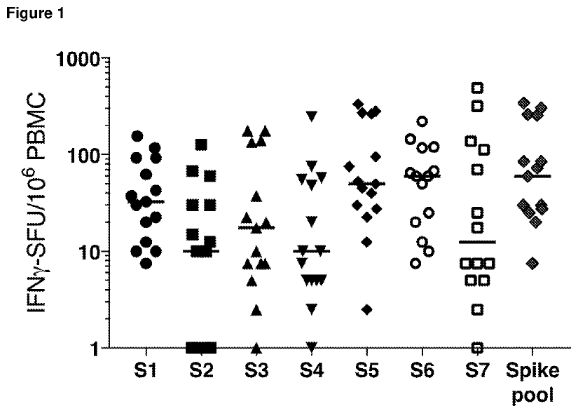

Four SARS-CoV-2 peptide pools of 15-mers (Tables 1-4) covering NP (NP-1, NP-2), membrane (M), and one pool of 55 peptides covering the most immunogenic regions of Spike (S)(FIG. 1) were generated. Spike is a long protein with 1276 amino acids, so it requires 253 15-mer peptides overlapping by 10 amino acids to cover the whole protein, thus 7 pools of about 40 peptides. To reduce the number of peptides pools to test, we selected a single “Spike pool” comprised of 55 peptides. For the selection, all sequences of published SARS-CoV-1 epitopes (wwwdotiedbdotorg; positive assays only, T cells assays, host: human) were aligned with the library of Spike-SARS-CoV-2 15-mers. The 15-mer peptides cover the homologue sequence of the described SARS-CoV-1 epitope sequences. In addition, we added the 15-mer peptides that cover the predicted SARS-CoV-2 Spike epitopes published by Grifoni et al., [Cell Host Microbe. 27(4): 671-680 (2020)]. The 55 peptides cover 40.5% of the Spike protein. The frequency of reactive cells to the selected Spike pool (red) was compared to the 7 pools of 15-mers overlapping by 10 amino acids covering together the entire Spike protein (S1-S7) in 15 COVID-19 convalescents. It would be understood that the invention is not limited to use of pools having specific peptide sequences disclosed herein, and that pools comprising peptides corresponding to a shift of one or only a few amino acids along the virus protein sequence may generate useful diagnostic data, given there are overlaps in the peptides.

TABLE 1
Summary of Peptide Pool M.

| POOL M | | | |
|---|---|---|---|
| Peptide | | | SEQ ID |
| Number | A.A Sequence | A.A Position | Number |
| M_1 | MADSNGTITVEELKK | 1-15 | 1 |
| M_2 | GTITVEELKKLLEQW | 6-20 | 2 |
| M_3 | EELKKLLEQWNLVIG | 11-25 | 3 |
| M_4 | LLEQWNLVIGFLFLT | 16-30 | 4 |
| M_5 | NLVIGFLFLTWICLL | 21-35 | 5 |
| M_6 | FLFLTWICLLQFAYA | 26-40 | 6 |
| M_7 | WICLLQFAYANRNRF | 31-45 | 7 |
| M_8 | QFAYANRNRFLYIIK | 36-50 | 8 |
| M_9 | NRNRFLYIIKLIFLW | 41-55 | 9 |
| M_10 | LYIIKLIFLWLLWPV | 46-60 | 10 |
| M_11 | LIFLWLLWPVTLACF | 51-65 | 11 |
| M_12 | LLWPVTLACFVLAAV | 56-70 | 12 |
| M_13 | TLACFVLAAVYRINW | 61-75 | 13 |
| M_14 | VLAAVYRINWITGGI | 66-80 | 14 |
| M_15 | YRINWITGGIAIAMA | 71-85 | 15 |
| M_16 | ITGGIAIAMACLVGL | 76-90 | 16 |
| M_17 | AIAMACLVGLMWLSY | 81-95 | 17 |
| M_18 | CLVGLMWLSYFIASF | 86-100 | 18 |
| M_19 | MWLSYFIASFRLFAR | 91-105 | 19 |
| M_20 | FIASFRLFARTRSMW | 96-110 | 20 |
| M_21 | RLFARTRSMWSFNPE | 101-115 | 21 |
| M_22 | TRSMWSFNPETNILL | 106-120 | 22 |
| M_23 | SFNPETNILLNVPLH | 111-125 | 23 |
| M_24 | TNILLNVPLHGTILT | 116-130 | 24 |
| M_25 | NVPLHGTILTRPLLE | 121-135 | 25 |
| M_26 | GTILTRPLLESELVI | 126-140 | 26 |
| M_27 | RPLLESELVIGAVIL | 131-145 | 27 |
| M_28 | SELVIGAVILRGHLR | 136-150 | 28 |
| M_29 | GAVILRGHLRIAGHH | 141-155 | 29 |
| M_30 | RGHLRIAGHHLGRCD | 146-160 | 30 |
| M_31 | IAGHHLGRCDIKDLP | 151-165 | 31 |
| M_32 | LGRCDIKDLPKEITV | 156-170 | 32 |
| M_33 | IKDLPKEITVATSRT | 161-175 | 33 |
| M_34 | KEITVATSRTLSYYK | 166-180 | 34 |
| M_35 | ATSRTLSYYKLGASQ | 171-185 | 35 |
| M_36 | LSYYKLGASQRVAGD | 176-190 | 36 |
| M_37 | LGASQRVAGDSGFAA | 181-195 | 37 |
| M_38 | RVAGDSGFAAYSRYR | 186-200 | 38 |
| M_39 | SGFAAYSRYRIGNYK | 191-205 | 39 |
| M_40 | YSRYRIGNYKLNTDH | 196-210 | 40 |
| M_41 | IGNYKLNTDHSSSSD | 201-215 | 41 |
| M_42 | LNTDHSSSSDNIALL | 206-220 | 42 |
| M_43 | SSSSDNIALLVQ | 211-222 | 43 |

TABLE 2
Summary of Peptide Pool NP-1.

| POOL | | | |
|---|---|---|---|
| NP-1 | | | |
| Peptide | | A.A | SEQ ID |
| Number | A.A Sequence | Position | Number |
| NP_1 | MSDNGPQNQRNAPRI | 1-15 | 44 |
| NP_2 | PQNQRNAPRITFGGP | 6-20 | 45 |
| NP_3 | NAPRITFGGPSDSTG | 11-25 | 46 |
| NP_4 | TFGGPSDSTGSNQNG | 16-30 | 47 |
| NP_5 | SDSTGSNQNGERSGA | 21-35 | 48 |
| NP_6 | SNQNGERSGARSKQR | 26-40 | 49 |
| NP_7 | ERSGARSKQRRPQGL | 31-45 | 50 |
| NP_8 | RSKQRRPQGLPNNTA | 36-50 | 51 |
| NP_9 | RPQGLPNNTASWFTA | 41-55 | 52 |
| NP_10 | PNNTASWFTALTQHG | 46-60 | 53 |
| NP_11 | SWFTALTQHGKEDLK | 51-65 | 54 |
| NP_12 | LTQHGKEDLKFPRGQ | 56-70 | 55 |
| NP_13 | KEDLKFPRGQGVPIN | 61-75 | 56 |
| NP_14 | FPRGQGVPINTNSSP | 66-80 | 57 |
| NP_15 | GVPINTNSSPDDQIG | 71-85 | 58 |
| NP_16 | TNSSPDDQIGYYRRA | 76-90 | 59 |
| NP_17 | DDQIGYYRRATRRIR | 81-95 | 60 |
| NP_18 | YYRRATRRIRGGDGK | 86-100 | 61 |
| NP_19 | TRRIRGGDGKMKDLS | 91-105 | 62 |
| NP_20 | GGDGKMKDLSPRWYF | 96-110 | 63 |
| NP_21 | MKDLSPRWYFYYLGT | 101-115 | 64 |
| NP_22 | PRWYFYYLGTGPEAG | 106-120 | 65 |
| NP_23 | YYLGTGPEAGLPYGA | 111-125 | 66 |
| NP_24 | GPEAGLPYGANKDGI | 116-130 | 67 |
| NP_25 | LPYGANKDGIIWVAT | 121-135 | 68 |
| NP_26 | NKDGIIWVATEGALN | 126-140 | 69 |
| NP_27 | IWVATEGALNTPKDH | 131-145 | 70 |
| NP_28 | EGALNTPKDHIGTRN | 136-150 | 71 |
| NP_29 | TPKDHIGTRNPANNA | 141-155 | 72 |
| NP_30 | IGTRNPANNAAIVLQ | 146-160 | 73 |
| NP_31 | PANNAAIVLQLPQGT | 151-165 | 74 |
| NP_32 | AIVLQLPQGTTLPKG | 156-170 | 75 |
| NP_33 | LPQGTTLPKGFYAEG | 161-175 | 76 |
| NP_34 | TLPKGFYAEGSRGGS | 166-180 | 77 |
| NP_35 | FYAEGSRGGSQASSR | 171-185 | 78 |
| NP_36 | SRGGSQASSRSSSRS | 176-190 | 79 |
| NP_37 | QASSRSSSRSRNSSR | 181-195 | 80 |
| NP_38 | SSSRSRNSSRNSTPG | 186-200 | 81 |
| NP_39 | RNSSRNSTPGSSRGT | 191-205 | 82 |
| NP_40 | NSTPGSSRGTSPARM | 196-210 | 83 |
| NP_41 | SSRGTSPARMAGNGG | 201-215 | 84 |

TABLE 3
Summary of Peptide Pool NP-2.

| POOL | | | |
|---|---|---|---|
| NP-2 | | | |
| Peptide | | A.A | SEQ ID |
| Number | A.A Sequence | Position | Number |
| NP_42 | SPARMAGNGGDAALA | 206-220 | 85 |
| NP_43 | AGNGGDAALALLLLD | 211-225 | 86 |
| NP_44 | DAALALLLLDRLNQL | 216-230 | 87 |
| NP_45 | LLLLDRLNQLESKMS | 221-235 | 88 |
| NP_46 | RLNQLESKMSGKGQQ | 226-240 | 89 |
| NP_47 | ESKMSGKGQQQQGQT | 231-245 | 90 |
| NP_48 | GKGQQQQGQTVTKKS | 236-250 | 91 |
| NP_49 | QQGQTVTKKSAAEAS | 241-255 | 92 |
| NP_50 | VTKKSAAEASKKPRQ | 246-260 | 93 |
| NP_51 | AAEASKKPRQKRTAT | 251-265 | 94 |
| NP_52 | KKPRQKRTATKAYNV | 256-270 | 95 |
| NP_53 | KRTATKAYNVTQAFG | 261-275 | 96 |
| NP_54 | KAYNVTQAFGRRGPE | 266-280 | 97 |
| NP_55 | TQAFGRRGPEQTQGN | 271-285 | 98 |
| NP_56 | RRGPEQTQGNFGDQE | 276-290 | 99 |
| NP_57 | QTQGNFGDQELIRQG | 281-295 | 100 |
| NP_58 | FGDQELIRQGTDYKH | 286-300 | 101 |
| NP_59 | LIRQGTDYKHWPQIA | 291-305 | 102 |
| NP_60 | TDYKHWPQIAQFAPS | 296-310 | 103 |
| NP_61 | WPQIAQFAPSASAFF | 301-315 | 104 |
| NP_62 | QFAPSASAFFGMSRI | 306-320 | 105 |
| NP_63 | ASAFFGMSRIGMEVT | 311-325 | 106 |
| NP_64 | GMSRIGMEVTPSGTW | 316-330 | 107 |
| NP_65 | GMEVTPSGTWLTYTG | 321-335 | 108 |
| NP_66 | PSGTWLTYTGAIKLD | 326-340 | 109 |
| NP_67 | LTYTGAIKLDDKDPN | 331-345 | 110 |
| NP_68 | AIKLDDKDPNFKDQV | 336-350 | 111 |
| NP_69 | DKDPNFKDQVILLNK | 341-355 | 112 |
| NP_70 | FKDQVILLNKHIDAY | 346-360 | 113 |
| NP_71 | ILLNKHIDAYKTFPP | 351-365 | 114 |
| NP_72 | HIDAYKTFPPTEPKK | 356-370 | 115 |
| NP_73 | KTFPPTEPKKDKKKK | 361-375 | 116 |
| NP_74 | TEPKKDKKKKADETQ | 366-380 | 117 |
| NP_75 | DKKKKADETQALPQR | 371-385 | 118 |
| NP_76 | ADETQALPQRQKKQQ | 376-390 | 119 |
| NP_77 | ALPQRQKKQQTVTLL | 381-395 | 120 |
| NP_78 | QKKQQTVTLLPAADL | 386-400 | 121 |
| NP_79 | TVTLLPAADLDDFSK | 391-405 | 122 |
| NP_80 | PAADLDDFSKQLQQS | 396-410 | 123 |
| NP_81 | DDFSKQLQQSMSSAD | 401-415 | 124 |
| NP_82 | QLQQSMSSADSTQA | 406-419 | 125 |

TABLE 4
Summary of Peptide Pool SP.

| POOL | | | |
|---|---|---|---|
| SP | | | |
| Peptide | | A.A | SEQ ID |
| Number | A.A Sequence | Position | Number |
| SP_21 | IRGWIFGTTLDSKTQ | 101-115 | 126 |
| SP_22 | FGTTLDSKTQSLLIV | 106-120 | 127 |
| SP_34 | CTFEYVSQPFLMDLE | 166-180 | 128 |
| SP_35 | VSQPFLMDLEGKQGN | 171-185 | 129 |
| SP_48 | TRFQTLLALHRSYLT | 236-250 | 130 |
| SP_49 | LLALHRSYLTPGDSS | 241-255 | 131 |
| SP_50 | RSYLTPGDSSSGWTA | 246-260 | 132 |
| SP_59 | CALDPLSETKCTLKS | 291-305 | 133 |
| SP_60 | LSETKCTLKSFTVEK | 296-310 | 134 |
| SP_61 | CTLKSFTVEKGIYQT | 301-315 | 135 |
| SP_62 | FTVEKGIYQTSNFRV | 306-320 | 136 |
| SP_63 | GIYQTSNFRVQPTES | 311-325 | 137 |
| SP_70 | RFASVYAWNRKRISN | 346-360 | 181 |
| SP_71 | YAWNRKRISNCVADY | 351-365 | 182 |
| SP_75 | SASFSTFKCYGVSPT | 371-385 | 138 |
| SP_76 | TFKCYGVSPTKLNDL | 376-390 | 139 |
| SP_85 | YNYKLPDDFTGCVIA | 421-435 | 140 |
| SP_88 | WNSNNLDSKVGGNYN | 436-450 | 141 |
| SP_89 | LDSKVGGNYNYLYRL | 441-455 | 142 |
| SP_90 | GGNYNYLYRLFRKSN | 446-460 | 143 |
| SP_91 | YLYRLFRKSNLKPFE | 451-465 | 144 |
| SP_92 | FRKSNLKPFERDIST | 456-470 | 145 |
| SP_93 | LKPFERDISTEIYQA | 461-475 | 146 |
| SP_106 | GPKKSTNLVKNKCVN | 526-540 | 147 |
| SP_107 | TNLVKNKCVNFNFNG | 531-545 | 148 |
| SP_109 | FNFNGLTGTGVLTES | 541-555 | 149 |
| SP_110 | LTGTGVLTESNKKFL | 546-560 | 150 |
| SP_130 | RAGCLIGAEHVNNSY | 646-660 | 151 |
| SP_131 | IGAEHVNNSYECDIP | 651-665 | 152 |
| SP_138 | SVASQSIIAYTMSLG | 686-700 | 153 |
| SP_139 | SIIAYTMSLGAENSV | 691-705 | 154 |
| SP_140 | TMSLGAENSVAYSNN | 696-710 | 155 |
| SP_150 | STECSNLLLQYGSFC | 746-760 | 156 |
| SP_151 | NLLLQYGSFCTQLNR | 751-765 | 157 |
| SP_156 | KNTQEVFAQVKQIYK | 776-790 | 158 |
| SP_157 | VFAQVKQIYKTPPIK | 781-795 | 159 |
| SP_158 | KQIYKTPPIKDFGGF | 786-800 | 160 |
| SP_159 | TPPIKDFGGFNFSQI | 791-805 | 161 |
| SP_161 | NFSQILPDPSKPSKR | 801-815 | 162 |
| SP_167 | AGFIKQYGDCLGDIA | 831-845 | 163 |
| SP_168 | QYGDCLGDIAARDLI | 836-850 | 164 |
| SP_179 | GAALQIPFAMQMAYR | 891-905 | 165 |
| SP_181 | QMAYRFNGIGVTQNV | 901-915 | 166 |
| SP_182 | FNGIGVTQNVLYENQ | 906-920 | 167 |
| SP_188 | DSLSSTASALGKLQD | 936-950 | 168 |
| SP_189 | TASALGKLQDVVNQN | 941-955 | 169 |
| SP_192 | AQALNTLVKQLSSNF | 956-970 | 170 |
| SP_196 | VLNDILSRLDKVEAE | 976-990 | 171 |
| SP_200 | LITGRLQSLQTYVTQ | 996-1010 | 172 |
| SP_203 | QLIRAAEIRASANLA | 1011-1025 | 173 |
| SP_204 | AEIRASANLAATKMS | 1016-1030 | 174 |
| SP_212 | APHGVVFLHVTYVPA | 1056-1070 | 175 |
| SP_221 | HWFVTQRNFYEPQII | 1101-1115 | 176 |
| SP_237 | KEIDRLNEVAKNLNE | 1181-1195 | 177 |
| SP_238 | LNEVAKNLNESLIDL | 1186-1200 | 178 |
| SP_239 | KNLNESLIDLQELGK | 1191-1205 | 179 |
| SP_244 | IWLGFIAGLIAIVMV | 1216-1230 | 180 |

### Example 2

### Sample Processing and Testing

### Human Samples

Whole blood samples (8 ml) were collected from individuals who were in contact with SARS-CoV-2 and tested antibody positive (Abbot test/anti-NP antibody), or were positive in a surrogate virus neutralization assay (sVNT) at the time of the T cell test (symptomatic n=35; asymptomatic n=73), or were healthy donors (n=51) without any history of SARS-CoV-2 contact and negative for anti-NP SARS-CoV-2 antibodies.

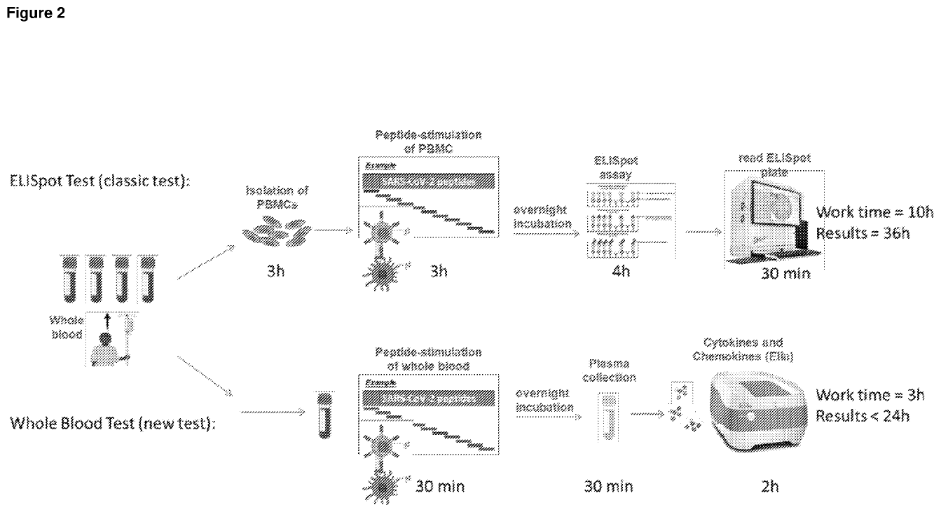

Schematic representations of suitable assays of the invention are presented in FIG. 2.

### ELISpot Testing of PBMCs

SARS-CoV-2-specific T cells were tested as described previously [Le Bert N, et al., Nature 584(7821): 457-62 (2020)]. Briefly, peripheral blood mononuclear cells (PBMCs) were isolated from 8 ml whole blood by density-gradient centrifugation using Ficoll-Paque™ (Sigma-Aldrich). Isolated PBMC were either studied directly or cryopreserved and stored in liquid nitrogen until used in the assays.

ELISpot plates (Millipore) were coated with human IFNγ antibody (1-D1K, Mabtech; 5 μg/ml) overnight at 4° C. Then, 4×10⁵ PBMCs were seeded per well and stimulated for 18 h with the different pools of SARS-CoV-2 peptides (2 μg/ml final concentration per peptide) described in Tables 1-4. For stimulation with peptide matrix pools or single peptides, a concentration of 5 μg/ml was used. Subsequently, the plates were developed with human biotinylated IFNγ detection antibody (7-B6-1, Mabtech; 1:2,000), followed by incubation with streptavidin-AP (Mabtech) and KPL BCIP/NBT Phosphatase Substrate (SeraCare). Spot forming units (SFU) were quantified with ImmunoSpot. To quantify positive peptide-specific responses, the highest number of spots of the unstimulated (or DMSO stimulated) wells was subtracted from the peptide-stimulated wells, and the results expressed as SFU/10⁶ PBMCs. We excluded the results if negative control wells had >30 SFU/10⁶ PBMCs or positive control wells (phorbol 12-myristate 13-acetate/ionomycin) were negative.

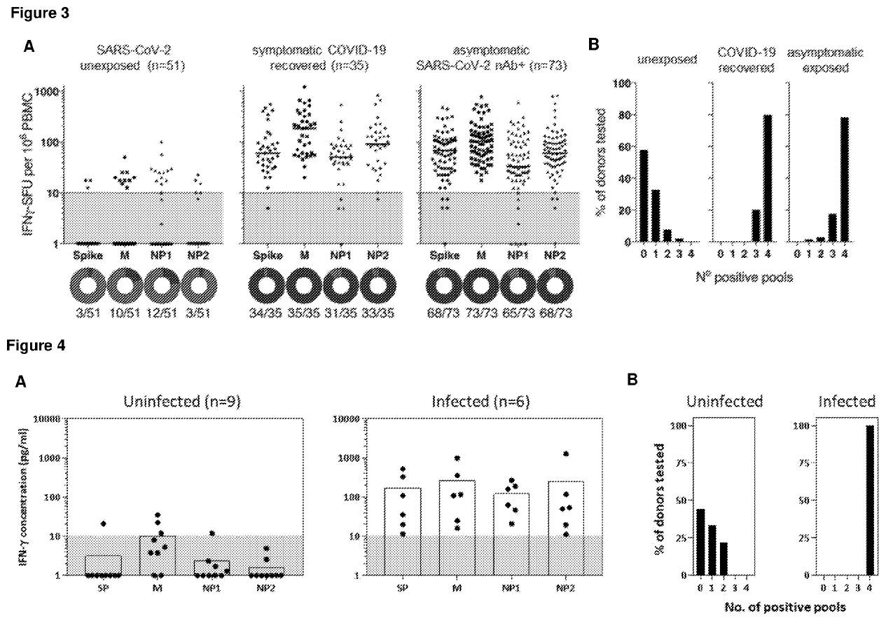

The ELISPOT assays showed that patients who have been infected by SARS-CoV-2 and cleared the virus up to 3 months ago have T cells that are reactive to peptide pools covering Membrane, Nucleoprotein and Spike (FIG. 3A). In contrast, individuals who are antibody anti-NP negative and without a history of SARS-CoV-2 infection (healthy donors) present only occasional responses to 1-2 peptide pools (FIG. 3B).

### Whole Blood Testing

Whole blood was isolated from each subject and tested within 24 hours after blood draw. 400 μl aliquots were separately mixed with 100 μl RPMI containing each of the SARS-CoV-2 peptide pools (2 μg/ml final concentration per peptide) or a DMSO control and incubated for a period extending overnight, to allow activation of responsive T cells. A plasma fraction was isolated from each of the incubated samples (about 30 minutes) and the level of cytokines in the sample measured using an EIIa™ multi-analyte ELISA machine (ProteinSimple, CA, USA).

It would be understood that the parameters described in the example may be changed and still achieve a valid assay. For example, the ratio of blood to RMPI media can range from 100% blood to 50% blood; 100 μl aliquots or more than 400 μl might be used, but a larger blood sample would be required to test multiple peptide pools. RPMI may be exchanged with other cell culture media. The final concentration of peptides in an assay mixture may be from 1 to 5 μg/ml. The control sample may contain DMSO or may be an unstimulated blood sample. The incubation period may range between about 6-24 h.

Having showed that the distinct peptide pools are able to define individuals that were recently in contact with SARS-CoV-2 (FIG. 3), we then demonstrated that the SARS-CoV-2 T cell response profile can also be delineated through the direct activation of antigen-specific T cells in whole blood using the same peptide pools and measuring the secreted cytokines in the plasma. We activated whole blood obtained from uninfected individuals (n=9) and individuals who have been infected with SARS-CoV-2 (n=6), as confirmed by the detection of anti-NP antibodies or by SARS-CoV-2 sVNT assays, using the respective peptide pools and measured the concentration of IFN-γ in the plasma collected after 24 hours.

SARS-CoV-2 reactive T cells in infected individuals can be detected by quantifying the amount of secreted IFN-γ after the direct addition of the peptide pools into whole blood (FIG. 4A). Similar to the results obtained with the ELISPOT assay, uninfected individuals occasionally have 1-2 responding peptide pools while infected individuals are simultaneously reactive to all peptide pools tested (FIG. 4B).

qPCR and dqPCR Testing of PBMCs

The complexity of quantifying the presence of virus-specific T cell has so far prevented large scale studies of the cellular immune response to viral infection or, more recently, the vaccine. To address this problem, we have implemented a qPCR-based rapid T cell Activation (qTACT) assay, based on in vitro stimulation of whole blood samples with a pool of viral peptides covering the spike or other SARS-CoV-2 viral proteins (i.e., nucleoprotein [N]) [Le Bert, N., et al. Nature 584: 457-462 (2020); Kalimuddin S, et al., Med (N Y). 2021 Jun. 11; 2(6):682-688.e4. doi: 10.1016/j.medj.2021.04.003; Le Bert, N. et al., J Exp Med 218(5):e20202617 (2021)], followed by direct amplification of IFN-γ or IL-2 (directly produced by SARS-CoV-2 antigen-specific T cells) or CXCL10, a molecule expressed by monocytes in response to T cell activation. A further technical implementation of the assay allows quantification of T cell immunity directly from blood, bypassing the need for red blood cell (RBC) lysis or RNA purification. We call this latter assay direct qPCR-based rapid T cell Activation (dqTACT) assay

### Summary of Methods

1. Collect blood (sodium heparin collection tube) & stimulate (320 μl blood+80 μl RPMI/peptide) overnight.
2. The next morning, collect 25 μl of serum in Eppendorf tubes for ELLA/ELISA (freeze −80° C.).
3. Replace serum with 25 μl of RPMI.
4. Vortex or pipette up and down aggressively.
5. Split remaining sample into two more tubes:
a. Tube A (whole blood—dqTACT): 180 μl blood+540 μl buffer A (process immediately or freeze −80° C.).
b. Tube B (RNA—qTACT): 180 μl blood+180 μl RNA/DNA shield+3.6 μl blood proteinase K (incubate at room temperature for 30 minutes then process immediately or freeze −80° C.).
i. To extract, add Trizol reagent (1:1) then proceed with Direct-zol 96 extraction kit (Zymo).

Primers and probes should be resuspended at 100 μM and stored at −20° C. Keep probes away from light when working with them.

TABLE 5
Primer & probe sequences
(for qTACT or dqTACT)

| | | SEQ |
|---|---|---|
| Primer/ | | ID |
| probe | Primer/probe sequence | No |
| ACTIN | /5HEX/TCATCCATG/ZEN/ | 455 |
| probe | GTGAGCTGGCGG/3IABkFQ/ | |
| HEX | | |
| CXCL10 | /56-FAM/AGTGGCATT/ZEN/ | 456 |
| probe | CAAGGAGTACCTCTCTCT/ | |
| FAM | 3IABkFQ/ | |
| ACTIN | CCTTGCACATGCCGGAG | 457 |
| primer 1 | | |
| ACTIN | ACAGAGCCTCGCCTTTG | 458 |
| primer 2 | | |
| CXCL10 | CCATTCTGATTTGCTGCCTTATC | 459 |
| primer 1 | | |
| CXCL10 | TACTAATGCTGATGCAGGTACAG | 460 |
| primer 2 | | |

dqTACT PCR 1× Mix:

10 μl SCRIPT Direct RT-qPCR ProbesMaster mix; 0.1 μl ACTIN primer 1; 0.1 μl ACTIN primer 2; 0.1 μl CXCL10 primer 1; 0.1 μl CXCL10 primer 2; 0.05 μl ACTIN probe (HEX); 0.05 μl CXCL10 probe (FAM); 2 μl blood/buffer A mixture; 7.5 μl PEC-1

qTACT PCR 1× Mix:

5 μl TaqPath 1 Step Multiplex Master Mix (no ROX); 0.1 μl ACTIN primer 1; 0.1 μl ACTIN primer 2; 0.1 μl CXCL10 primer 1; 0.1 μl CXCL10 primer 2; 0.05 μl ACTIN probe (HEX); 0.05 μl CXCL10 probe (FAM); 5 μl RNA; 9.5 μl PEC-1

dqTACT Run Conditions (Hyris bCUBE ONLY):
Reverse transcription: 53° C. for 15 minutes
Initial denaturation: 95° C. for 5 minutes
PCR (X45 cycles):
95° C. 15 seconds
60° C. 30 seconds
   qTACT Run Conditions (Hyris bCUBE or CFX96/384)
UNG incubation: 25° C. for 2 minutes
Reverse transcription: 53° C. for 10 minutes
Initial denaturation: 95° C. for 2 minutes
PCR (X45 cycles):
95° C. for 15 seconds
60° C. for 30 seconds

### Protocol Descriptions

Whole Blood Culture with SARS-CoV-2 Peptide Pools

320 μl of whole blood drawn on the same day into sodium heparin tubes (BD) were mixed with 80 μl RPMI and stimulated with pools of SARS-CoV-2 peptides (S or NP; 2 μg/ml) or DMSO control at 37° C. After 15-17 hours of stimulation, the supernatant (plasma) was collected and stored at −80° C. until quantification of cytokines.

qTACT Assay

Samples used for RNA extraction were diluted 1:1 with RNA/DNA shield (Zymo) and incubated at room temperature with proteinase K at a 1:100 dilution (20 mg/ml stock). Samples were then frozen at −80° C. until RNA extraction could be performed. Samples stored in RNA/DNA shield were thawed at room temperature prior to RNA extraction. Samples were vortexed and mixed with Trizol reagent (Life Technologies) at a 1:1 dilution. After vortexing, samples were processed using the Direct-zol 96 well extraction kit (Zymo) as per the manufacturer's instructions. Eluted RNA was diluted in TE buffer, aliquoted, and stored at −80° C. or used immediately for qPCR analysis. Real-time quantification was performed on a BioRad CFX96/CFX384 or Hyris bCUBE 2.0. 5 μl of diluted RNA was used with the TaqPath 1-Step Multiplex MasterMix (Applied Biosystems) and primers/probes targeting ACTIN (internal control) and other target genes, as described.

dqTACT Assay

Samples used for direct amplification from whole blood were diluted 1:3 with Buffer A and stored at −80° C. or used immediately for qPCR analysis. 2 μl of diluted whole blood was mixed with SCRIPT Direct RT-qPCR ProbesMaster (Jena Bioscience) and primers/probes targeting ACTIN (internal control) and other target genes, as described. Quantification was performed using the Hyris bCUBE 2.0.

Reagents:
Buffer A: 2% Tween-20 in RNAse free water
SCRIPT Direct RT-qPCR ProbesMaster mix: worldwidewebdotjenabiosciencedotcom/molecular-biology/reversetranscription-rt-pcr/direct-rt-qpcr-robust-amplification/pcr-528-script-direct-rt-qpcr-probesmaster
PEC-1: worldwidewebdotklentaqdotcom/products/pcr-enhancer-cocktail-1
TaqPath 1 Step Multiplex Master Mix: worldwidewebdotthermofisherdotcom/order/catalog/product/A28526#/A28526

### Algorithm Representing T Cell Response Profile

The present study demonstrates that distinct SARS-CoV-2 peptide pools are able to define the individuals that were recently infected with SARS-CoV-2. This T cell response profile can be evaluated using other laboratory techniques capable of detecting T cell activation after peptide stimulation, including the direct activation of antigen-specific T cells in whole blood. A proposed algorithm to interpret the SARS-CoV-2 T cell response profile is summarized in Table 6.

Table 6 summarizes the interpretation of the SARS-CoV-2 T cell response profile in Example 1. When 50% or more of the pools (thus 2, 3 or 4 out of 4) are positive, the subject is categorized as having SARS-COV2 specific T cells induced by SARS-COV-2 infection (thus previously or currently SARS-COV-2 infected).

TABLE 6
Proposed algorithm to interpret SARS-
CoV-2 T cell response profile

| No. of positive pools | Spike | M | NP1 | NP2 | Interpretation |
|---|---|---|---|---|---|
| 4 | + | + | + | + | Infected |
| 3 | − | + | + | + | Infected |
| | + | − | + | + | |
| | + | + | − | + | |
| | + | + | + | − | |
| 2 | + | + | − | − | Infected |
| | + | − | + | − | |
| | + | − | − | + | |
| | − | + | + | − | |
| | − | + | − | + | |
| | − | − | + | + | |
| 1 | + | − | − | − | Uninfected |
| | − | + | − | − | |
| | − | − | + | − | |
| | − | − | − | + | |
| 0 | − | − | − | − | Uninfected |

### Example 3

### Impact of Variants of Concern on T Cells Induced by SARS-CoV-2 Wuhan Infection or SARS-CoV-2 Vaccines

Different variants of concern (VOC) of SARS-CoV-2 have replaced world-wide the original SARS-CoV-2 Wuhan isolate. These VOCs are characterized by amino acid substitutions that provide biological advantages like increased infectivity or escape humoral (antibodies) but also cellular (T cells) immunity. We have designed a method based on specific combination of peptide pools covering both different SARS-CoV-2 proteins (i.e., Spike) and the corresponding regions affected by the amino acid mutations that are used to stimulate T cells, The results of this multiple stimulation strategy define with accuracy the impact of these amino acid mutations on the SARS-CoV-2-specific T cells induced by infection with Wuhan strain or by vaccination with the present vaccines based on the Wuhan strain. Peptide pools directed to non-conserved regions of the Wuhan strain variants are shown in Tables 8 to 19, while the sequences of the regions of the Wuhan strain that correspond to the regions of the Delta variant (B.1.617.2) are shown in Table 7.

The inventors present here as an example the method to analyze the impact of AA mutations present in Spike in different VOCs (using Delta variant as an example) on the SPIKE specific T cells induced by vaccination.

TABLE 7
Peptide sequences of non-conserved regions
in Wuhan strain corresponding to Delta
mutation peptides shown in Table 8.

| POOL | | | |
|---|---|---|---|
| SP | | | |
| Peptide | | A.A | SEQ ID |
| Number | A.A Sequence | Position | Number |
| SP_002 | VLLPLVSSQCVNLTT | 6-20 | 461 |
| SP_003 | VSSQCVNLTTRTQLP | 11-25 | 462 |
| SP_004 | VNLTTRTQLPPAYTN | 16-30 | 463 |
| SP_027 | CEFQFCNDPFLGVYY | 131-145 | 464 |
| SP_028 | CNDPFLGVYYHKNNK | 136-150 | 465 |
| SP_029 | LGVYYHKNNKSWMES | 141-155 | 466 |
| SP_030 | HKNNKSWMESEFRVY | 146-160 | 467 |
| SP_031 | SWMESEFRVYSSANN | 151-165 | 468 |
| SP_032 | EFRVYSSANNCTFEY | 156-170 | 469 |
| SP_089 | LDSKVGGNYNYLYRL | 441-455 | 142 |
| SP_090 | GGNYNYLYRLFRKSN | 446-460 | 143 |
| SP_091 | YLYRLFRKSNLKPFE | 451-465 | 144 |
| SP_094 | RDISTEIYQAGSTPC | 466-480 | 470 |
| SP_095 | EIYQAGSTPCNGVEG | 471-485 | 471 |
| SP_096 | GSTPCNGVEGFNCYF | 476-490 | 472 |
| SP_121 | GTNTSNQVAVLYQDV | 601-615 | 473 |
| SP_122 | NQVAVLYQDVNCTEV | 606-620 | 474 |
| SP_123 | LYQDVNCTEVPVAIH | 611-625 | 475 |
| SP_135 | CASYQTQTNSPRRAR | 671-685 | 476 |
| SP_136 | TQTNSPRRARSVASQ | 676-690 | 477 |
| SP_137 | PRRARSVASQSIIAY | 681-695 | 478 |
| SP_188 | DSLSSTASALGKLQD | 936-950 | 168 |
| SP_189 | TASALGKLQDVVNQN | 941-955 | 169 |
| SP_190 | GKLQDVVNQNAQALN | 946-960 | 479 |

TABLE 8
Peptide sequences of non-conserved regions
in SEQUENCE_21A (Delta) (B.1.617.2)

| POOL | | | |
|---|---|---|---|
| SP | | | |
| Peptide | | A.A | SEQ ID |
| Number | A.A Sequence | Position | Number |
| SP_002 | VLLPLVSSQCVNLRT | 6-20 | 183 |
| SP_003 | VSSQCVNLRTRTQLP | 11-25 | 184 |
| SP_004 | VNLRTRTQLPPAYTN | 16-30 | 185 |
| SP_027 | CEFQFCNDPFLDVYY | 131-145 | 186 |
| SP_028 | CNDPFLDVYYHKNNK | 136-150 | 187 |
| SP_029 | LDVYYHKNNKSWMES | 141-155 | 188 |
| SP_030 | HKNNKSWMESGVYSS | 146-160 | 189 |
| SP_031 | SWMESGVYSSANNCT | 151-165 | 190 |
| SP_032 | GVYSSANNCTFEYVS | 156-170 | 191 |
| SP_089 | LDSKVGGNYNYRYRL | 441-455 | 192 |
| SP_090 | GGNYNYRYRLFRKSN | 446-460 | 193 |
| SP_091 | YRYRLFRKSNLKPFE | 451-465 | 194 |
| SP_094 | RDISTEIYQAGSKPC | 466-480 | 195 |
| SP_095 | EIYQAGSKPCNGVEG | 471-485 | 196 |
| SP_096 | GSKPCNGVEGFNCYF | 476-490 | 197 |
| SP_121 | GTNTSNQVAVLYQGV | 601-615 | 198 |
| SP_122 | NQVAVLYQGVNCTEV | 606-620 | 199 |
| SP_123 | LYQGVNCTEVPVAIH | 611-625 | 200 |
| SP_135 | CASYQTQTNSRRRAR | 671-685 | 201 |
| SP_136 | TQTNSRRRARSVASQ | 676-690 | 202 |
| SP_137 | RRRARSVASQSIIAY | 681-695 | 203 |
| SP_188 | DSLSSTASALGKLQN | 936-950 | 204 |
| SP_189 | TASALGKLQNVVNQN | 941-955 | 205 |
| SP_190 | GKLQNVVNQNAQALN | 946-960 | 206 |

TABLE 9
Peptide sequences of non-conserved regions
in SEQUENCE_20I (Alpha, V1) (B.1.1.7)

| POOL | | | |
|---|---|---|---|
| SP | | | |
| Peptide | | A.A | SEQ ID |
| Number | A.A Sequence | Position | Number |
| SP_012 | LPFFSNVTWFHAISG | 56-70 | 207 |
| SP_013 | NVTWFHAISGTNGTK | 61-75 | 208 |
| SP_014 | HAISGTNGTKRFDNP | 66-80 | 209 |
| SP_027 | CEFQFCNDPFLGVYH | 131-145 | 210 |
| SP_028 | CNDPFLGVYHKNNKS | 136-150 | 211 |
| SP_029 | LGVYHKNNKSWMESE | 141-155 | 212 |
| SP_095 | EIYQAGSTPCNGVKG | 471-485 | 213 |
| SP_096 | GSTPCNGVKGFNCYF | 476-490 | 214 |
| SP_097 | NGVKGFNCYFPLQPY | 481-495 | 215 |
| SP_098 | FNCYFPLQPYGFQPT | 486-500 | 216 |
| SP_099 | PLQPYGFQPTYGVGY | 491-505 | 217 |
| SP_100 | GFQPTYGVGYQPYRV | 496-510 | 218 |
| SP_101 | YGVGYQPYRVVVLSF | 501-515 | 219 |
| SP_112 | NKKFLPFQQFGRDID | 556-570 | 220 |
| SP_113 | PFQQFGRDIDDTTDA | 561-575 | 221 |
| SP_114 | GRDIDDTTDAVRDPQ | 566-580 | 222 |
| SP_121 | GTNTSNQVAVLYQGV | 601-615 | 223 |
| SP_122 | NQVAVLYQGVNCTEV | 606-620 | 224 |
| SP_123 | LYQGVNCTEVPVAIH | 611-625 | 225 |
| SP_135 | CASYQTQTNSHRRAR | 671-685 | 226 |
| SP_136 | TQTNSHRRARSVASQ | 676-690 | 227 |
| SP_137 | HRRARSVASQSIIAY | 681-695 | 228 |
| SP_142 | AYSNNSIAIPINFTI | 706-720 | 229 |
| SP_143 | SIAIPINFTISVTTE | 711-725 | 230 |
| SP_144 | INFTISVTTEILPVS | 716-730 | 231 |
| SP_195 | GAISSVLNDILARLD | 971-985 | 232 |
| SP_196 | VLNDILARLDKVEAE | 976-990 | 233 |
| SP_197 | LARLDKVEAEVQIDR | 981-995 | 234 |
| SP_222 | QRNFYEPQIITTHNT | 1106-1120 | 235 |
| SP_223 | EPQIITTHNTFVSGN | 1111-1125 | 236 |
| SP_224 | TTHNTFVSGNCDVVI | 1116-1130 | 237 |
| SP-237 | KEIDRLNEVANNLNE | 1181-1195 | 238 |
| SP_238 | LNEVANNLNESLIDL | 1186-1200 | 239 |
| SP_239 | NNLNESLIDLQELGK | 1191-1205 | 240 |

TABLE 10
Peptide sequences of non-conserved regions
in SEQUENCE_20H (Beta, V2) (B.1.351)

| POOL | | | |
|---|---|---|---|
| SP | | | |
| Peptide | | A.A | SEQ ID |
| Number | A.A Sequence | Position | Number |
| SP_002 | VLLPLVSSQCVNFTT | 6-20 | 241 |
| SP_003 | VSSQCVNFTTRTQLP | 11-25 | 242 |
| SP_004 | VNFTTRTQLPPAYTN | 16-30 | 243 |
| SP_014 | HAIHVSGTNGTKRFA | 66-80 | 244 |
| SP_015 | SGTNGTKRFANPVLP | 71-85 | 245 |
| SP_016 | TKRFANPVLPFNDGV | 76-90 | 246 |
| SP_041 | FKIYSKHTPINLVRG | 201-215 | 247 |
| SP_042 | KHTPINLVRGLPQGF | 206-220 | 248 |
| SP_043 | NLVRGLPQGFSALEP | 211-225 | 249 |
| SP_047 | IGINITRFQTLHRSY | 231-245 | 250 |
| SP_048 | TRFQTLHRSYLTPGD | 236-250 | 251 |
| SP_049 | LHRSYLTPGDSSSGW | 241-255 | 252 |
| SP_082 | EVRQIAPGQTGNIAD | 406-420 | 253 |
| SP_083 | APGQTGNIADYNYKL | 411-425 | 254 |
| SP_084 | GNIADYNYKLPDDFT | 416-430 | 255 |
| SP_095 | EIYQAGSTPCNGVKG | 471-485 | 256 |
| SP_096 | GSTPCNGVKGFNCYF | 476-490 | 257 |
| SP_097 | NGVKGFNCYFPLQSY | 481-495 | 258 |
| SP_099 | PLQSYGFQPTYGVGY | 491-505 | 259 |
| SP_100 | GFQPTYGVGYQPYRV | 496-510 | 260 |
| SP_101 | YGVGYQPYRVVVLSF | 501-515 | 261 |
| SP_121 | GTNTSNQVAVLYQGV | 601-615 | 262 |
| SP_122 | NQVAVLYQGVNCTEV | 606-620 | 263 |
| SP_123 | LYQGVNCTEVPVAIH | 611-625 | 264 |
| SP_139 | SIIAYTMSLGVENSV | 691-705 | 265 |
| SP_140 | TMSLGVENSVAYSNN | 696-710 | 266 |
| SP_141 | VENSVAYSNNSIAIP | 701-715 | 267 |

TABLE 11
Peptide sequences of non-conserved regions
in SEQUENCE_20J (Gamma, V3) (P.1)

| POOL | | | |
|---|---|---|---|
| SP | | | |
| Peptide | | A.A | SEQ ID |
| Number | A.A Sequence | Position | Number |
| SP_002 | VLLPLVSSQCVNFTN | 6-20 | 268 |
| SP_003 | VSSQCVNFTNRTQLP | 11-25 | 269 |
| SP_004 | VNFTNRTQLPSAYTN | 16-30 | 270 |
| SP_005 | RTQLPSAYTNSFTRG | 21-35 | 271 |
| SP_006 | SAYTNSFTRGVYYPD | 26-40 | 272 |
| SP_026 | VVIKVCEFQFCNYPF | 126-140 | 273 |
| SP_027 | CEFQFCNYPFLGVYY | 131-145 | 274 |
| SP_028 | CNYPFLGVYYHKNNK | 136-150 | 275 |
| SP_036 | LMDLEGKQGNFKNLS | 176-190 | 276 |
| SP_037 | GKQGNFKNLSEFVFK | 181-195 | 277 |
| SP_038 | FKNLSEFVFKNIDGY | 186-200 | 278 |
| SP_082 | EVRQIAPGQTGTIAD | 406-420 | 279 |
| SP_083 | APGQTGTIADYNYKL | 411-425 | 280 |
| SP_084 | GTIADYNYKLPDDFT | 416-430 | 281 |
| SP_095 | EIYQAGSTPCNGVKG | 471-485 | 282 |
| SP_096 | GSTPCNGVKGFNCYF | 476-490 | 283 |
| SP_097 | NGVKGFNCYFPLQSY | 481-495 | 284 |
| SP_099 | PLQSYGFQPTYGVGY | 491-505 | 285 |
| SP_100 | GFQPTYGVGYQPYRV | 496-510 | 286 |
| SP_101 | YGVGYQPYRVVVLSF | 501-515 | 287 |
| SP_121 | GTNTSNQVAVLYQGV | 601-615 | 288 |
| SP_122 | NQVAVLYQGVNCTEV | 606-620 | 289 |
| SP_123 | LYQGVNCTEVPVAIH | 611-625 | 290 |
| SP_129 | NVFQTRAGCLIGAEY | 641-655 | 291 |
| SP_130 | RAGCLIGAEYVNNSY | 646-660 | 292 |
| SP_131 | IGAEYVNNSYECDIP | 651-665 | 293 |
| SP_204 | AEIRASANLAAIKMS | 1016-1030 | 294 |
| SP_205 | SANLAAIKMSECVLG | 1021-1035 | 295 |
| SP_206 | AIKMSECVLGQSKRV | 1026-1040 | 296 |
| SP_234 | LGDISGINASFVNIQ | 1166-1180 | 297 |
| SP_235 | GINASFVNIQKEIDR | 1171-1185 | 298 |
| SP_236 | FVNIQKEIDRLNEVA | 1176-1190 | 299 |

TABLE 12
Peptide sequences of non-conserved regions
in SEQUENCE_21B (Kappa) (B.1.617.1)

| POOL | | | |
|---|---|---|---|
| SP | | | |
| Peptide | | A.A | SEQ ID |
| Number | A.A Sequence | Position | Number |
| SP_017 | NPVLPFNDGVYFASI | 81-95 | 300 |
| SP_018 | FNDGVYFASIEKSNI | 86-100 | 301 |
| SP_019 | YFASIEKSNIIRGWI | 91-105 | 302 |
| SP_027 | CEFQFCNDPFLDVYY | 131-145 | 303 |
| SP_028 | CNDPFLDVYYHKNNK | 136-150 | 304 |
| SP_029 | LDVYYHKNNKSWMKS | 141-155 | 305 |
| SP_030 | HKNNKSWMKSEFRVY | 146-160 | 306 |
| SP_031 | SWMKSEFRVYSSANN | 151-165 | 307 |
| SP_089 | LDSKVGGNYNYRYRL | 441-455 | 308 |
| SP_090 | GGNYNYRYRLFRKSN | 446-460 | 309 |
| SP_091 | YRYRLFRKSNLKPFE | 451-465 | 310 |
| SP_095 | EIYQAGSTPCNGVQG | 471-485 | 311 |
| SP_096 | GSTPCNGVQGFNCYF | 476-490 | 312 |
| SP_097 | NGVQGFNCYFPLQSY | 481-495 | 313 |
| SP_121 | GTNTSNQVAVLYQGV | 601-615 | 314 |
| SP_122 | NQVAVLYQGVNCTEV | 606-620 | 315 |
| SP_123 | LYQGVNCTEVPVAIH | 611-625 | 316 |
| SP_135 | CASYQTQTNSRRRAR | 671-685 | 317 |
| SP_136 | TQTNSRRRARSVASQ | 676-690 | 318 |
| SP_137 | RRRARSVASQSIIAY | 681-695 | 319 |
| SP_213 | VFLHVTYVPAHEKNF | 1061-1075 | 320 |
| SP_214 | TYVPAHEKNFTTAPA | 1066-1080 | 321 |
| SP_215 | HEKNFTTAPAICHDG | 1071-1085 | 322 |

TABLE 13
Peptide sequences of non-conserved regions
in SEQUENCE_20B/S.484K (P.2)

| POOL | | | |
|---|---|---|---|
| SP | | | |
| Peptide | | A.A | SEQ ID |
| Number | A.A Sequence | Position | Number |
| SP_095 | EIYQAGSTPCNGVKG | 471-485 | 323 |
| SP_096 | GSTPCNGVKGFNCYF | 476-490 | 324 |
| SP_097 | NGVKGFNCYFPLQSY | 481-495 | 325 |
| SP_121 | GTNTSNQVAVLYQGV | 601-615 | 326 |
| SP_122 | NQVAVLYQGVNCTEV | 606-620 | 327 |
| SP_123 | LYQGVNCTEVPVAIH | 611-625 | 328 |
| SP_234 | LGDISGINASFVNIQ | 1166-1180 | 329 |
| SP_235 | GINASFVNIQKEIDR | 1171-1185 | 330 |
| SP_236 | FVNIQKEIDRLNEVA | 1176-1190 | 331 |

TABLE 14
Peptide sequences of non-conserved regions
in SEQUENCE_21C (Epsilon) (B.1.427/9)

| POOL | | | |
|---|---|---|---|
| SP | | | |
| Peptide | | A.A | SEQ ID |
| Number | A.A Sequence | Position | Number |
| SP_001 | MFVFLVLLPLVSIQC | 1-15 | 332 |
| SP_002 | VLLPLVSIQCVNLTT | 6-20 | 333 |
| SP_003 | VSIQCVNLTTRTQLP | 11-25 | 334 |
| SP_029 | LGVYYHKNNKSCMES | 141-155 | 335 |
| SP_030 | HKNNKSCMESEFRVY | 146-160 | 336 |
| SP_031 | SCMESEFRVYSSANN | 151-165 | 337 |
| SP_089 | LDSKVGGNYNYRYRL | 441-455 | 338 |
| SP_090 | GGNYNYRYRLFRKSN | 446-460 | 339 |
| SP_091 | YRYRLFRKSNLKPFE | 451-465 | 340 |
| SP_121 | GTNTSNQVAVLYQGV | 601-615 | 341 |
| SP_122 | NQVAVLYQGVNCTEV | 606-620 | 342 |
| SP_123 | LYQGVNCTEVPVAIH | 611-625 | 343 |

TABLE 15
Peptide sequences of non-conserved regions
in SEQUENCE_21D (Eta) (B.1.525)

| POOL | | | |
|---|---|---|---|
| SP | | | |
| Peptide | | A.A | SEQ ID |
| Number | A.A Sequence | Position | Number |
| SP_009 | KVFRSSVLHSTRDLF | 41-55 | 344 |
| SP_010 | SVLHSTRDLFLPFFS | 46-60 | 345 |
| SP_011 | TRDLFLPFFSNVTWF | 51-65 | 346 |
| SP_012 | LPFFSNVTWFHVISG | 56-70 | 347 |
| SP 013 | NVTWFHVISGTNGTK | 61-75 | 348 |
| SP_014 | HVISGTNGTKRFDNP | 66-80 | 349 |
| SP_027 | CEFQFCNDPFLGVYH | 131-145 | 350 |
| SP_028 | CNDPFLGVYHKNNKS | 136-150 | 351 |
| SP-029 | LGVYHKNNKSWMESE | 141-155 | 352 |
| SP_095 | EIYQAGSTPCNGVKG | 471-485 | 353 |
| SP_096 | GSTPCNGVKGFNCYF | 476-490 | 354 |
| SP_097 | NGVKGFNCYFPLQSY | 481-495 | 355 |
| SP_121 | GTNTSNQVAVLYQGV | 601-615 | 356 |
| SP_122 | NQVAVLYQGVNCTEV | 606-620 | 357 |
| SP-123 | LYQGVNCTEVPVAIH | 611-625 | 358 |
| SP_134 | IGAGICASYQTHTNS | 666-680 | 359 |
| SP_135 | CASYQTHTNSPRRAR | 671-685 | 360 |
| SP_136 | THTNSPRRARSVASQ | 676-690 | 361 |
| SP_176 | ALLAGTITSGWTLGA | 876-890 | 362 |
| SP_177 | TITSGWTLGAGAALQ | 881-895 | 363 |
| SP_178 | WTLGAGAALQIPFAM | 886-900 | 364 |

TABLE 16
Peptide sequences of non-conserved regions
in SEQUENCE_21F (Iota) (B.1.526)

| POOL | | | |
|---|---|---|---|
| SP | | | |
| Peptide | | A.A | SEQ ID |
| Number | A.A Sequence | Position | Number |
| SP_001 | MFVFFVLLPLVSSQC | 1-15 | 365 |
| SP_017 | NPVLPFNDGVYFASI | 81-95 | 366 |
| SP_018 | FNDGVYFASIEKSNI | 86-100 | 367 |
| SP_019 | YFASIEKSNIIRGWI | 91-105 | 368 |
| SP_049 | LLALHRSYLTPGGSS | 241-255 | 369 |
| SP_050 | RSYLTPGGSSSGWTA | 246-260 | 370 |
| SP_051 | PGGSSSGWTAGAAAY | 251-265 | 371 |
| SP_095 | EIYQAGSTPCNGVKG | 471-485 | 372 |
| SP_096 | GSTPCNGVKGFNCYF | 476-490 | 373 |
| SP_097 | NGVKGFNCYFPLQSY | 481-495 | 374 |
| SP_121 | GTNTSNQVAVLYQGV | 601-615 | 375 |
| SP_122 | NQVAVLYQGVNCTEV | 606-620 | 376 |
| SP_123 | LYQGVNCTEVPVAIH | 611-625 | 377 |
| SP_139 | SIIAYTMSLGVENSV | 691-705 | 378 |
| SP_140 | TMSLGVENSVAYSNN | 696-710 | 379 |
| SP_141 | VENSVAYSNNSIAIP | 701-715 | 380 |

TABLE 17
Peptide sequences of non-conserved regions
in SEQUENCE_21G (Lambda) (C.37)

| POOL | | | |
|---|---|---|---|
| SP | | | |
| Peptide | | A.A | SEQ ID |
| Number | A.A Sequence | Position | Number |
| SP_013 | NVTWFHAIHVSGTNV | 61-75 | 381 |
| SP_014 | HAIHVSGTNVIKRFD | 66-80 | 382 |
| SP_015 | SGTNVIKRFDNPVLP | 71-85 | 383 |
| SP_016 | IKRFDNPVLPFNDGV | 76-90 | 384 |
| SP_048 | TRFQTLLALHNSSSG | 236-250 | 385 |
| SP_049 | LLALHNSSSGWTAGA | 241-255 | 386 |
| SP_050 | NSSSGWTAGAAAYYV | 246-260 | 387 |
| SP_089 | LDSKVGGNYNYQYRL | 441-455 | 388 |
| SP_090 | GGNYNYQYRLFRKSN | 446-460 | 389 |
| SP_091 | YQYRLFRKSNLKPFE | 451-465 | 390 |
| SP_096 | GSTPCNGVEGFNCYS | 476-490 | 391 |
| SP_097 | NGVEGFNCYSPLQSY | 481-495 | 392 |
| SP_098 | FNCYSPLQSYGFQPT | 486-500 | 393 |
| SP_121 | GTNTSNQVAVLYQGV | 601-615 | 394 |
| SP_122 | NQVAVLYQGVNCTEV | 606-620 | 395 |
| SP_123 | LYQGVNCTEVPVAIH | 611-625 | 396 |
| SP_170 | ARDLICAQKFNGLNV | 846-860 | 397 |
| SP_171 | CAQKFNGLNVLPPLL | 851-865 | 398 |
| SP_172 | NGLNVLPPLLTDEMI | 856-870 | 399 |

TABLE 18
Peptide sequences of non-conserved regions
in SEQUENCE_21H (B.1.621)

| POOL SP | | | |
|---|---|---|---|
| Peptide | | A.A | SEQ ID |
| Number | A.A Sequence | Position | Number |
| SP_017 | NPVLPFNDGVYFASI | 81-95 | 400 |
| SP_018 | FNDGVYFASIEKSNI | 86-100 | 401 |
| SP_019 | YFASIEKSNIIRGWI | 91-105 | 402 |
| SP_027 | CEFQFCNDPFLGVSN | 131-145 | 403 |
| SP_028 | CNDPFLGVSNHKNNK | 136-150 | 404 |
| SP_029 | LGVSNHKNNKSWMES | 141-155 | 405 |
| SP_068 | CPFGEVFNATKFASV | 336-350 | 406 |
| SP_069 | VFNATKFASVYAWNR | 341-355 | 407 |
| SP_070 | KFASVYAWNRKRISN | 346-360 | 408 |
| SP_095 | EIYQAGSTPCNGVKG | 471-485 | 409 |
| SP_096 | GSTPCNGVKGFNCYF | 476-490 | 410 |
| SP_097 | NGVKGFNCYFPLQSY | 481-495 | 411 |
| SP_099 | PLQSYGFQPTYGVGY | 491-505 | 412 |
| SP_100 | GFQPTYGVGYQPYRV | 496-510 | 413 |
| SP_101 | YGVGYQPYRVVVLSF | 501-515 | 414 |
| SP_121 | GTNTSNQVAVLYQGV | 601-615 | 415 |
| SP_122 | NQVAVLYQGVNCTEV | 606-620 | 416 |
| SP_123 | LYQGVNCTEVPVAIH | 611-625 | 417 |
| SP_135 | CASYQTQTNSHRRAR | 671-685 | 418 |
| SP_136 | TQTNSHRRARSVASQ | 676-690 | 419 |
| SP_137 | HRRARSVASQSIIAY | 681-695 | 420 |
| SP_188 | DSLSSTASALGKLQN | 936-950 | 421 |
| SP_189 | TASALGKLQNVVNQN | 941-955 | 422 |
| SP_190 | GKLQNVVNQNAQALN | 946-960 | 423 |

TABLE 19
Peptide sequences of non-conserved regions
in SEQUENCE_20A/S:126A (B.1.620)

| POOL SP | | | |
|---|---|---|---|
| Peptide | | A.A | SEQ ID |
| Number | A.A Sequence | Position | Number |
| SP_004 | VNLTTRTQLPSAYTN | 16-30 | 424 |
| SP_005 | RTQLPSAYTNSFTRG | 21-35 | 425 |
| SP_006 | SAYTNSFTRGVYYPD | 26-40 | 426 |
| SP_012 | LPFFSNVTWFHAISG | 56-70 | 427 |
| SP_013 | NVTWFHAISGTNGTK | 61-75 | 428 |
| SP_014 | HAISGTNGTKRFDNP | 66-80 | 429 |
| SP_024 | SLLIVNNATNAVIKV | 116-130 | 430 |
| SP_025 | NNATNAVIKVCEFQF | 121-135 | 431 |
| SP_026 | AVIKVCEFQFCNDPF | 126-140 | 432 |
| SP_027 | CEFQFCNDPFLGVYH | 131-145 | 433 |
| SP_028 | CNDPFLGVYHKNNKS | 136-150 | 434 |
| SP_029 | LGVYHKNNKSWMESE | 141-155 | 435 |
| SP_047 | IGINITRFQTLYRSY | 231-245 | 436 |
| SP_048 | TRFQTLYRSYLTPGD | 236-250 | 437 |
| SP_049 | LYRSYLTPGDSSSGW | 241-255 | 438 |
| SP_094 | RDISTEIYQAGNTPC | 466-480 | 439 |
| SP_095 | EIYQAGNTPCNGVKG | 471-485 | 440 |
| SP_096 | GNTPCNGVKGFNCYF | 476-490 | 441 |
| SP_097 | NGVKGFNCYFPLQSY | 481-495 | 442 |
| SP_121 | GTNTSNQVAVLYQGV | 601-615 | 443 |
| SP_122 | NQVAVLYQGVNCTEV | 606-620 | 444 |
| SP_123 | LYQGVNCTEVPVAIH | 611-625 | 445 |
| SP_135 | CASYQTQTNSHRRAR | 671-685 | 446 |
| SP_136 | TQTNSHRRARSVASQ | 676-690 | 447 |
| SP_137 | HRRARSVASQSIIAY | 681-695 | 448 |
| SP_204 | AEIRASANLAAIKMS | 1016-1030 | 449 |
| SP_205 | SANLAAIKMSECVLG | 1021-1035 | 450 |
| SP_206 | AIKMSECVLGQSKRV | 1026-1040 | 451 |
| SP_222 | QRNFYEPQIITTHNT | 1106-1120 | 452 |
| SP_223 | EPQIITTHNTFVSGN | 1111-1125 | 453 |
| SP_224 | TTHNTFVSGNCDVVI | 1116-1130 | 454 |

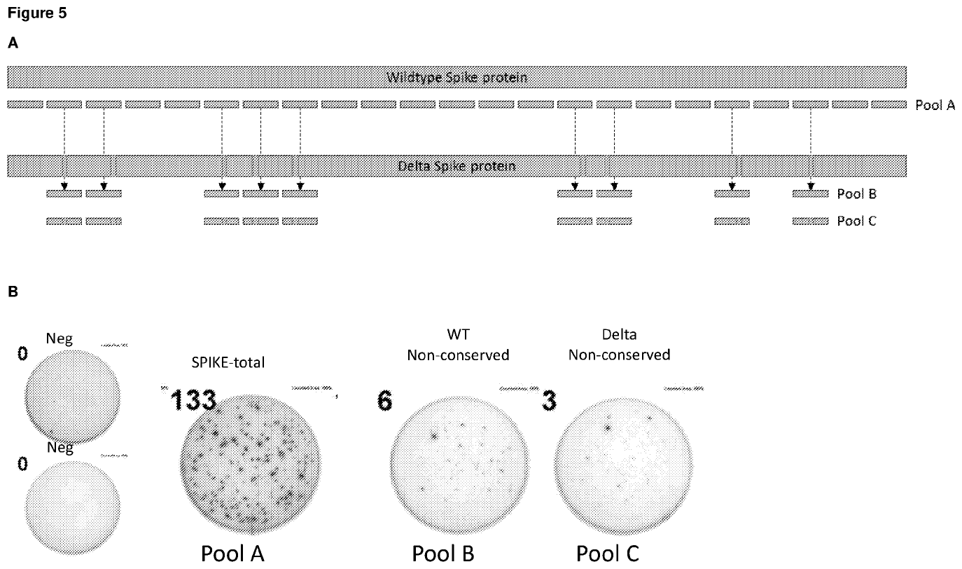

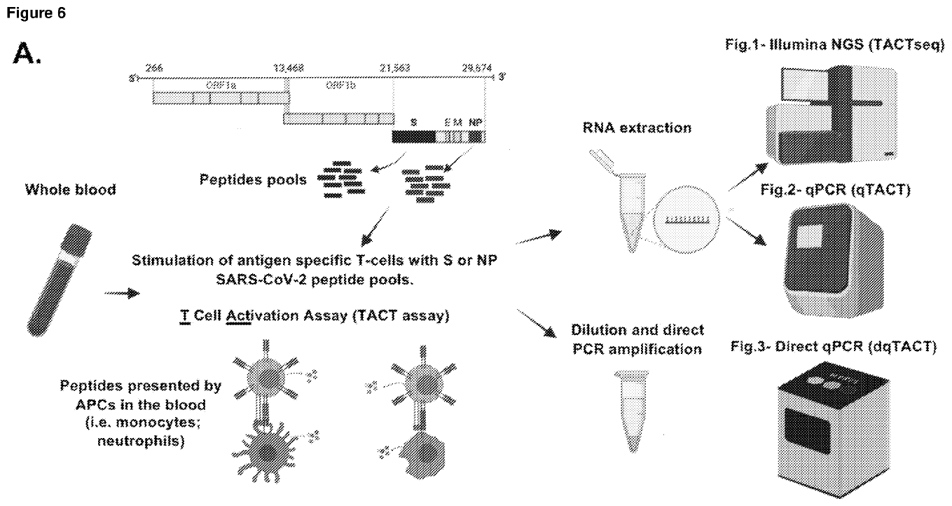

An embodiment is shown in schematic diagram FIG. 5A. The inventors designed peptide pools containing peptides that cover the whole Spike-Wuhan protein (Pool A; 253 peptides of 15 amino acids in length, overlapping adjacent peptides by 10 amino acids, derived from SEQ ID NO: 795) and the non-conserved Spike-Wuhan regions affected by mutations present in the delta variant (Pool B; Table 7). The third peptide pool (Pool C; Table 8) contains peptides from Pool B with the amino acid mutations present in the Spike-Delta.

These peptide pools can be, for example, used in a classical ELISPOT assay and thus used to stimulate PBMC of different vaccinated individuals (FIG. 5B). The number of spots obtained in each experiment is analyzed and utilized to derive in each single individual, the frequency of T cells directed towards the whole Spike (PBMC stimulated with peptide pool A), the frequency of T cells directed toward the non-conserved Spike-Wuhan region (PBMC stimulated with Pool B) and the frequency of T cells inhibited by AA mutations present in these mutated Spike-Delta region (PBMC stimulated with pool C). Overall, the test provides the estimation of the ability of T cells of a given individual to recognize the conserved and non-conserved region of different Spike proteins and the ability of mutations to inhibit the T cell response towards Spike. This experimental system can be done by utilizing different peptide pools covering other mutated SARS-CoV-2 proteins (i.e., NP, M).

Thus, the inventors can obtain a measurement of the alteration that the mutations present in VOCs can exert on total SARS-CoV-2 T cell response.

### Example 4

### PCR-Based Test to Detect Virus Cellular Immunity

The inventors show in this Example that qPCR can be used to quantify the presence of virus-specific T cells, based on ex vivo stimulation of whole blood samples with a pool of viral peptides covering the spike or other SARS-CoV-2 viral proteins (i.e. nucleoprotein [NP]), followed by direct amplification of IFN-γ or IL-2 (directly produced by SARS-CoV-2 antigen-specific T cells) or CXCL10, a molecule expressed by monocytes in response to T cell activation.

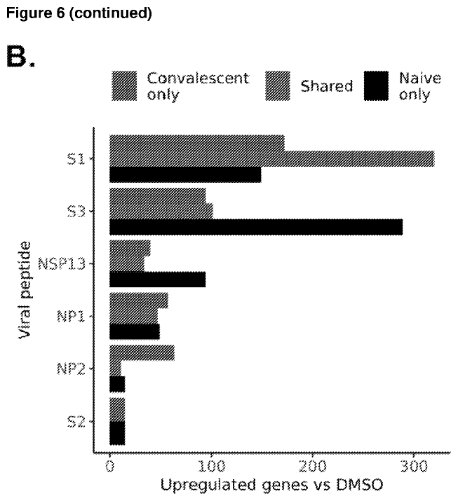

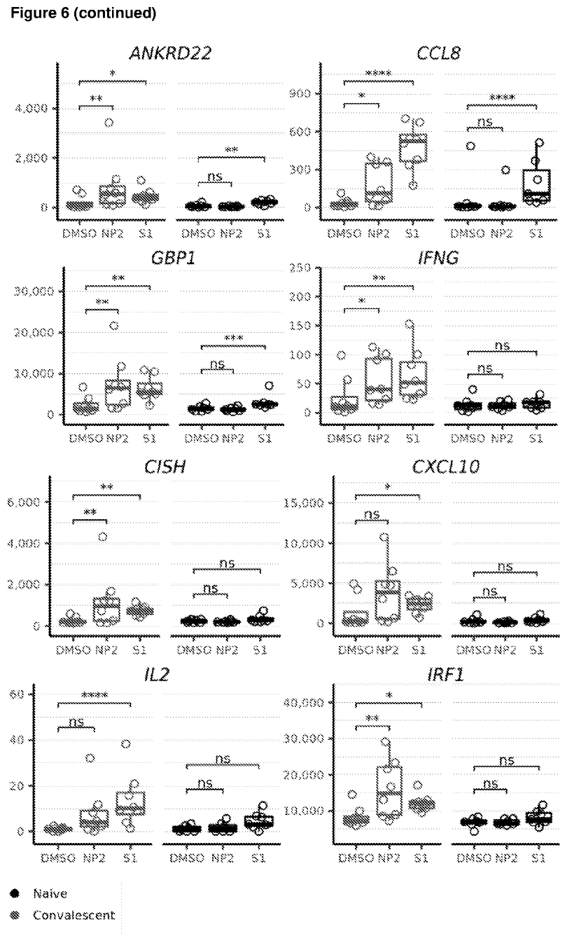

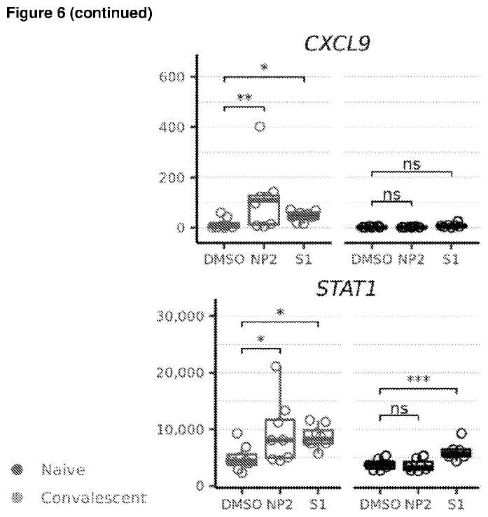

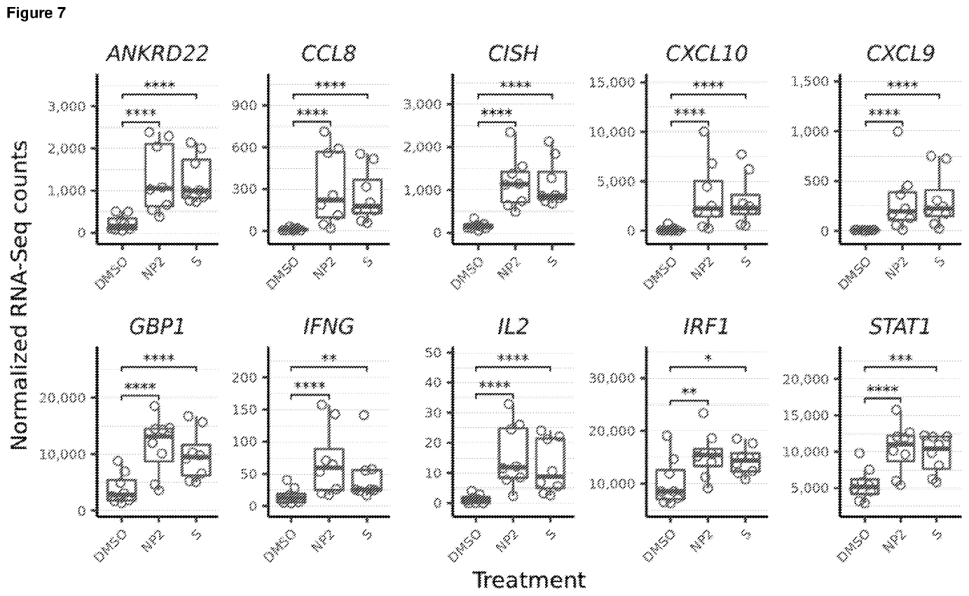

In order to select genes whose induction would correlate with the presence and activation of antigen specific T cells; we first evaluated the transcriptional profile of whole blood after overnight stimulation with SARS-CoV-2 peptide pools by RNA sequencing (FIG. 6A). This initial cohort consisted of 7 naïve and 11 COVID-19 convalescent subjects. Briefly, whole blood was incubated overnight with DMSO or multiple pools of SARS-CoV-2 peptides, including three distinct pools of the spike (S) protein, corresponding to the first 100 peptides covering the first 510 amino acids, and two distinct pools of the structural nucleocapsid protein (NP-1 and NP-2, Tables 2 and 3). The full 253 spike peptides were divided into 7 peptide pools of around 35˜ peptides each. The first 3 pools, comprising the first 100 peptides, cover the S1 chain of the spike protein. RNA was extracted from the cell pellet as described in Example 2, and subjected to Illumina single end sequencing (Koh, C. M. et al., Nature 523: 96-100 (2015))20. We then identified genes activated by viral peptides by performing a differential expression analysis between peptide-stimulated samples and untreated controls, across all subjects (FIG. 6B). Treatment with the S1 pool induced the largest changes in gene expression, with over 600 genes significantly upregulated (FDR<0.05, log 2FoldChange>1) across naïve and SARS-CoV-2 convalescent subjects. However, about half of these genes were shared across the two groups, suggesting that the transcriptional effects induced by this pool of peptides are not highly specific to previous exposure to SARS-CoV-2. NP2 treatment induced the most specific response, with 63 genes uniquely upregulated in convalescent individuals, and only 15 in naïve individuals and 11 shared between groups. Not surprisingly, these upregulated genes belonged to “cellular response to interferon gamma signaling”, “response to cytokine” and “Jak/Stat signaling” pathways. To narrow down a shortlist of candidates for further investigation by qPCR, we selected a panel of 10 genes that were significantly upregulated following either S1 or NP2 stimulation in convalescent subjects, but not as significantly in naïve subjects (FIG. 6C). Upregulation of these genes was further confirmed by RNA-seq in a validation cohort of eight COVID-19 convalescent subjects (FIG. 7).

qPCR

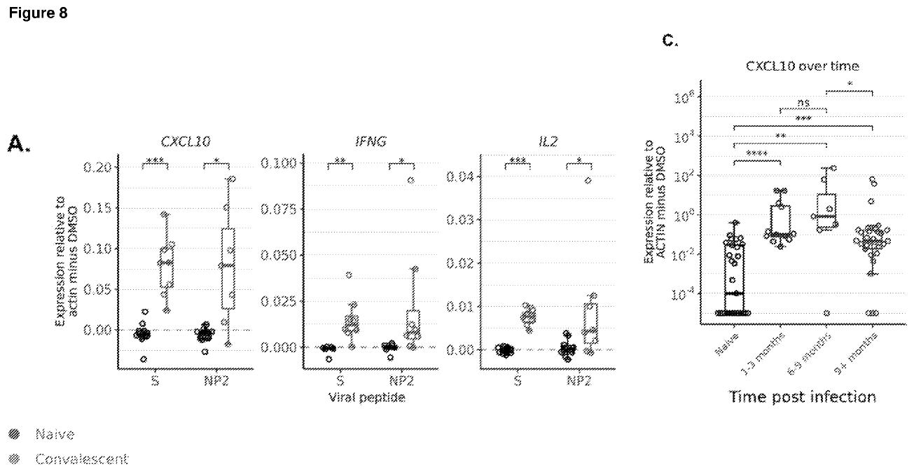

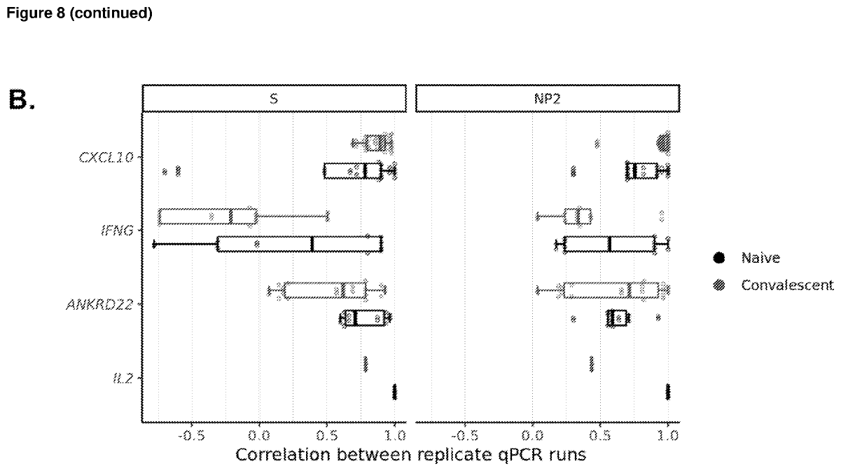

Next, we validated transcriptional induction of shortlisted genes by qPCR (as described in Example 2) in 11 naïve and 8 COVID-19 convalescent subjects. Out of all genes tested (FIG. 8A), we selected CXCL10, IFN-γ and IL2 for further studies. Between the three candidate genes, CXCL10 was not only the most reproducible, but it was the only gene that could accurately distinguish naïve and COVID-19 recovered individuals using multiple qPCR machines, including the QuantStudio 5 (Applied Biosystems), CFX96 (BioRad), CFX384 (BioRad), and bCUBE 2.0 (Hyris) (FIG. 8A-B). We observed that for IFN-γ and IL2, we were able to categorize patients if the assay was performed using the Hyris bCUBE. This instrument is a portable, 2-channel machine that is able to quantify up to 36 wells at a time.

To assess the reliability of the qTACT test, we compared CXCL10 and IFN-γ expression levels to IFN-γ cytokine secretion as quantified by ELLA in a larger cohort comprised of 89 subjects (43 naïve and 46 COVID-19 convalescent. For this cohort, only samples stimulated with DMSO (control) and the spike peptide pool (Table 4) were considered for downstream analysis. CXCL10 was selected as preferred because our previous data had confirmed its reliability and reproducibility when distinguishing between naïve and COVID-19 convalescent subjects. For this larger cohort, we chose to keep IFN-γ, despite its inferiority relative to CXCL10, to include a gene that is expressed by antigen-specific T cells and to directly correlate mRNA expression (qTACT) with IFN-γ protein secretion (ELLA). The cohort was recruited prior to vaccination and followed at day 10 and 20 after the first and second dose of the BNT162b2 vaccine and the data on IFN-γ an IL-2 cytokine secretion have been described elsewhere (Camara C., et al., bioRxiv 2021.03.22.436441; doi:/10.1101/2021.03.22.436441).

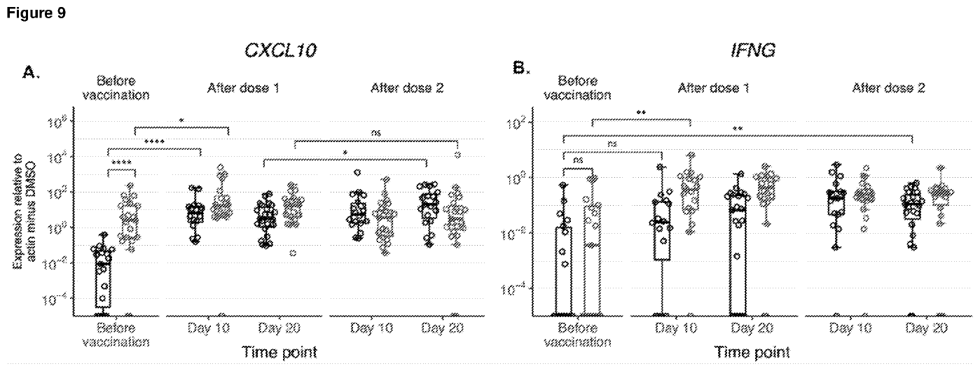

Compared to naïve subjects, COVID-19 recovered individuals had a higher median expression of CXCL10 prior to vaccination (2.53 [N=21] vs 0.0087 [N=19] in naïve subjects) (FIG. 9A). A similar trend was observed for IFN-γ levels (median pre-vaccination −0.00185 [N=18] in naïve and 0.0036 [N=19] in COVID-19 recovered subjects) but the difference was not statistically significant (FIG. 9B. Quantification of the spike-specific T cell response by both CXCL10 and IFN-γ qPCR 10 days after the first dose indicates that naïve subjects mount a weaker response compared to COVID-19 recovered individuals (FIG. 9A-B. These results are consistent with data obtained by direct IFN-γ and IL-2 cytokine quantification (Camara C., et al., bioRxiv 2021.03.22.436441; doi: 10.1101/2021.03.22.436441). The technical advantage over the use of ELISA or ELISPOT, is the ease of use of qPCR and, importantly, the internal normalization standard (i.e., ACTIN levels), which is absent in other more laborious methods of quantifying cellular immunity.

### A Second Vaccine Dose Increases CXCL10 and IFN-v Expression Levels in Naiive Subjects but not in COVID-19 Recovered Individuals

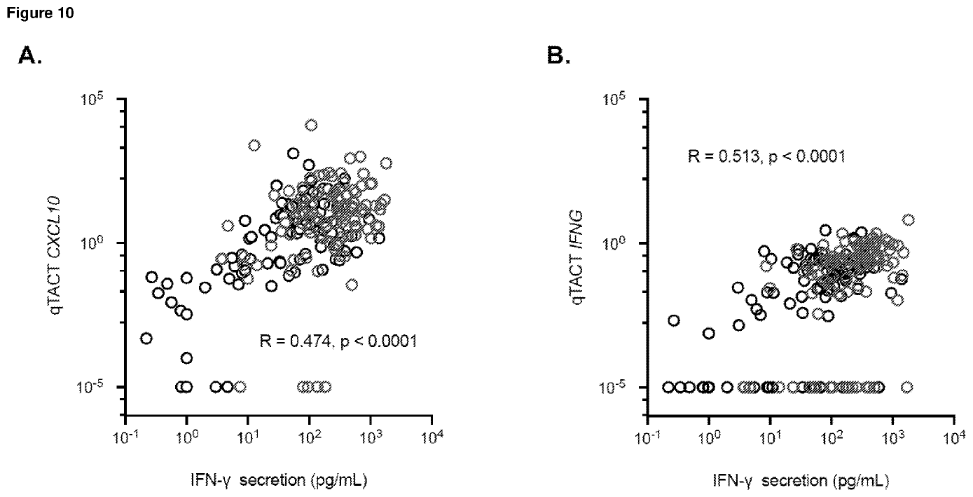

The effect of a second dose of the vaccine was next studied. Sampling on day 10 and 20 after a second dose confirmed the beneficial effects of the recall vaccine in naïve individuals who increase their CXCL10 and IFN-γ expression to significant levels. On the contrary, the second dose in COVID-19 recovered individuals did not have a boost effect (no significant increase in CXCL10 and IFN-γ levels) (FIG. 9A-B). These findings indicate that while naïve subjects significantly increase their cytokine production induced by SARS-CoV-2 spike protein after the second dose of the vaccine, COVID-19 recovered individuals do not further increase IFN-γ and CXCL10 levels following the standard regimen for COVID-19 vaccination, consistent with previously published work quantifying cytokine production (Camara C., et al., bioRxiv 2021.03.22.436441; doi: 10.1101/2021.03.22.436441; Tauzin, A. et al., bioRxiv. 2021 Mar. 18; 2021.03.18.435972. doi: 10.1101/2021.03.18.435972). Consistent with what we previously reported (Camara C., et al., bioRxiv 2021.03.22.436 441; doi: 10.1101/2021.03.22.43644116), both CXCL10 and IFN-γ mRNA levels induced by the spike peptide pool strongly correlate with IFN-γ cytokine quantification in the same cohort (Spearman r=0.474, and r=0.513, respectively; p<0.0001)(FIG. 10).

qPCR Quantification of CXCL10 as a Proxy for Cellular Immunity

We used the qPCR assay to quantify the levels of CXCL10, as a proxy for cellular immunity, over time. We assessed the level of T cell activation in a small cohort of COVID-19 convalescent subjects at different time points post infection. We observed that, while for some patients CXCL10 levels decreased to background levels after 9 months from SARS-CoV-2 infection, we were able to detect the activation of antigen specific T cells in the majority of subjects even 9-12 months post infection (FIG. 8C).

dqPCR

Blood samples were prepared as described in Example 2. To this end, following overnight incubation with DMSO control, or SARS-CoV-2 nucleoprotein or spike peptide pools (Table 4), we took 50 μl of blood, diluted it (1:3) to avoid PCR inhibition by anticoagulants (i.e., heparin), and loaded 2 μl directly onto a qPCR instrument (dqTACT)(FIG. 6A). For this cohort, we chose to once again include the nucleoprotein (NP-2, Table 3), this time to act as an additional negative control for vaccinated subjects. All of the vaccinated subjects that were selected had never been exposed to the virus, thus, we expected that they would not mount a cellular immune response to anything but the spike protein, upon which the mRNA vaccinations are based.

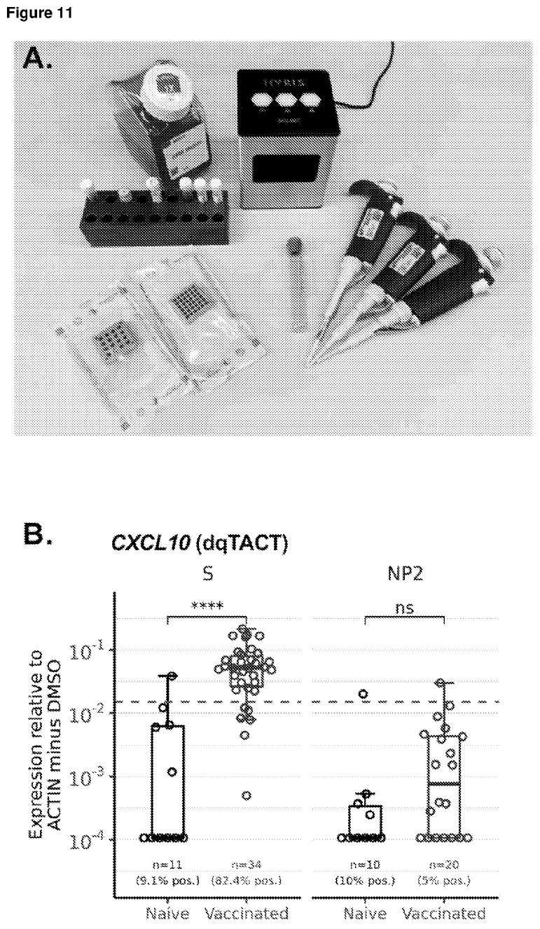

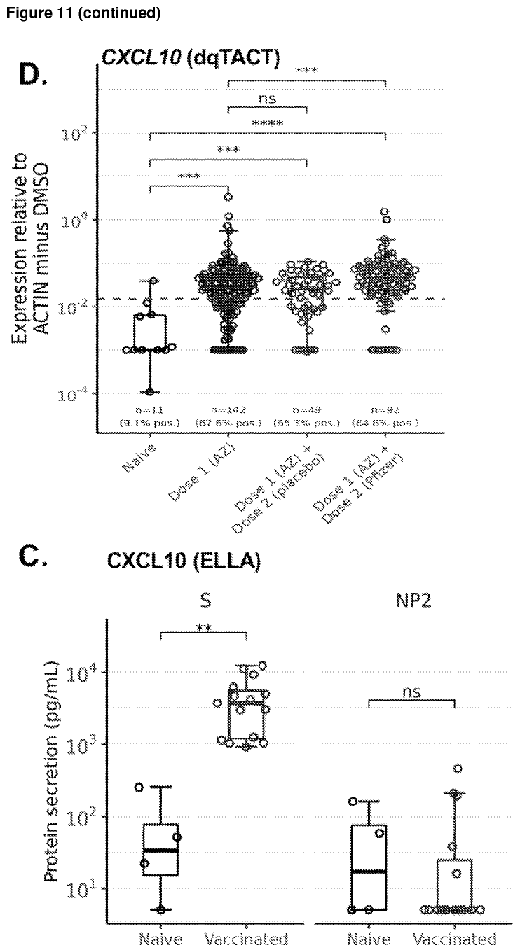

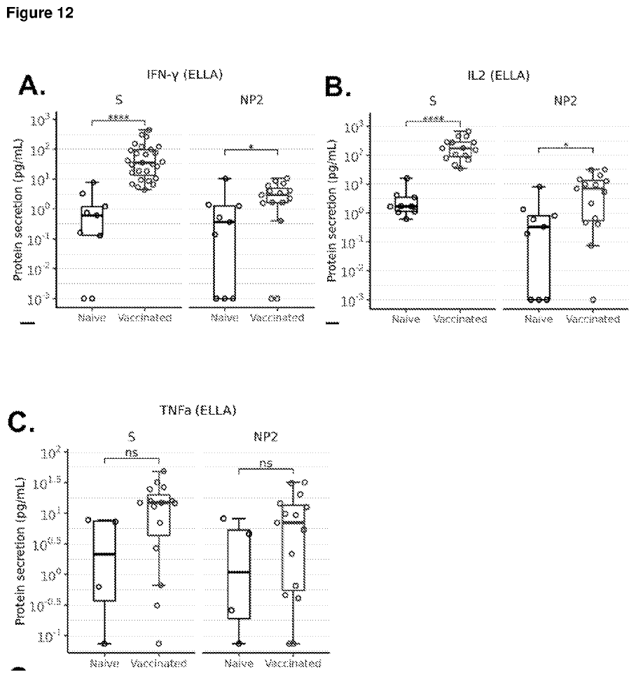

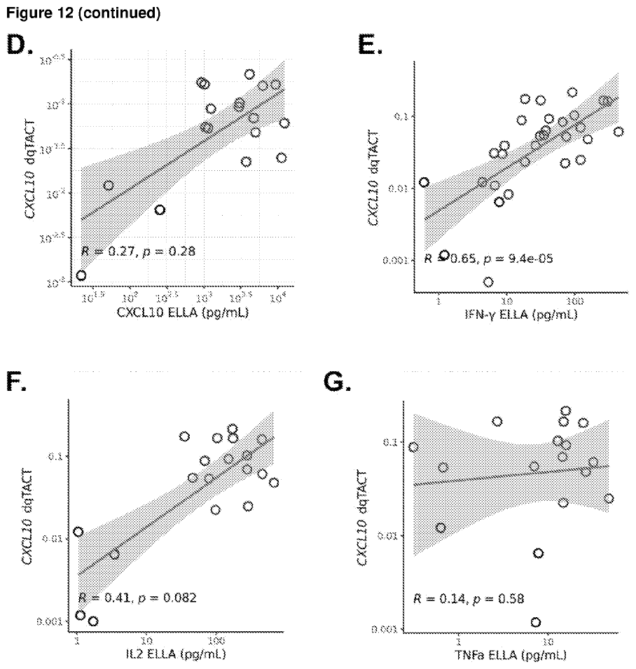

IFN-γ and CXCL10 were tested as potential readouts for the dqTACT assay (described in Example 2), being optimized for use on the Hyris bCUBE given its high range of detection compared to other tested instruments, the reduced cost, and the ease of assay set up (FIG. 11A). Our results showed that IFN-γ could not reliably stratify naïve and vaccinated individuals (data not shown), while CXCL10 did so robustly (FIG. 11B). Without being bound by theory, the low abundance of IFN-γ is likely due to the small number of antigen-specific T cells in whole blood, which are the direct source of IFN-gamma. In contrast, CXCL10, being an IFN-gamma-stimulated chemokine, is upregulated and expressed by a much higher percentage of cells (i.e. monocytes and neutrophils, which are roughly 5% and 60%, respectively, of all white cells in whole blood) (Ichikawa, A. et al., Am J Respir Crit Care Med 187: 65-77 (2013); Luster, A. D., Nature 315: 672-676 (1985)). Therefore, when taking only a small volume of blood, CXCL10 is not subject to sampling bias as is IFN-γ or other antigen-specific T cell transcripts (0.1% of all white cells in whole blood). In addition, we decided to compare the level of cytokine production in the same cohort. First, we quantified TNFa, CXCL10 (IP-10), IFN-γ and IL-2 by ELLA. All cytokines, except TNFa, successfully stratified naïve from vaccinated subjects (FIG. 11C and FIG. 12A-C), and correlate well with CXCL10 mRNA quantification obtained by the dqTACT assay (FIG. 12D-G).

A lack of rapid, accessible, and accurate diagnostic methods to quantify cellular immunity hinders long-term vaccination strategies and public health responses to global pandemics, such as the one being caused by SARS-CoV-2. Considering that diagnostic centers around the world have ramped up the setup of RT-qPCR based facilities, we developed a qPCR-based dqTACT assay, which is amenable to periodic and repeated testing of patient samples, as it requires only 1 ml of blood and a 24-hour turnaround time. Clinical validation of this assay in response to recent draft guidance from the United States Food and Drug Administration (FDA) and European Medicines Agency (EMA) is ongoing in a Clinical Laboratory Improvement Amendments (CLIA)-certified microbiology laboratory. First, we implemented a Next Generation Sequencing (NGS) approach. The pros of this approach, which could be further implemented by a targeted amplification panel of 15-20 genes, is the possibility of capturing the variability of the response and measure cytokines produced by both T cells and other myeloid cells in the blood. The cons are a longer turnaround time, a higher cost, and the need for skilled technical personnel.

Second, we developed a qPCR-based method (qTACT assay) on a 96 or 384 well platform (BioRad CFX). The advantages of this approach are the accuracy and sensitivity of qPCR probes, the opportunity to combine more than 2 fluorophores to measure the expression of 2-4 genes, and the scalability and potential automation of the process. The cons include a relatively longer processing time (48 hours per 200 samples), the need to purify RNA (by standard RNA-purification kits/columns), higher associated costs, and a certain level of technical skill (although less than that required for NGS).

Third, we optimized a direct qPCR-based method (dqTACT assay) on the HYRIS bCUBE platform. The advantages of this approach are the accuracy of qPCR probes, the increased accuracy of the bCUBE platform over other tested qPCR machines, and the reduced processing time/cost/skill required. Overall, this is an easy to implement protocol that requires minimal training of the operator, thus reducing technical errors. The cons include a relatively lower scalability (18 samples on the bCUBE as opposed to 48/192 samples on the CFX 96/384) and the limitation to a 2 fluorophore/2 genes detection system.

The derived profile of SARS-CoV-2-specific T cell activation by qTACT/dqTACT assays in different cohorts of naïve, infected or vaccinated individuals, will provide information about their level of SARS-CoV-2-specific cellular immunity.

### Example 5

### HBV Peptide Pools

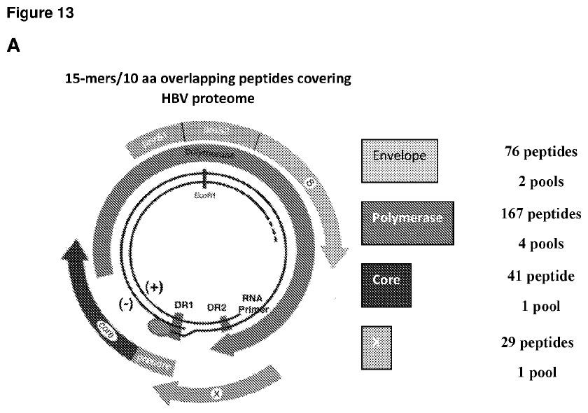

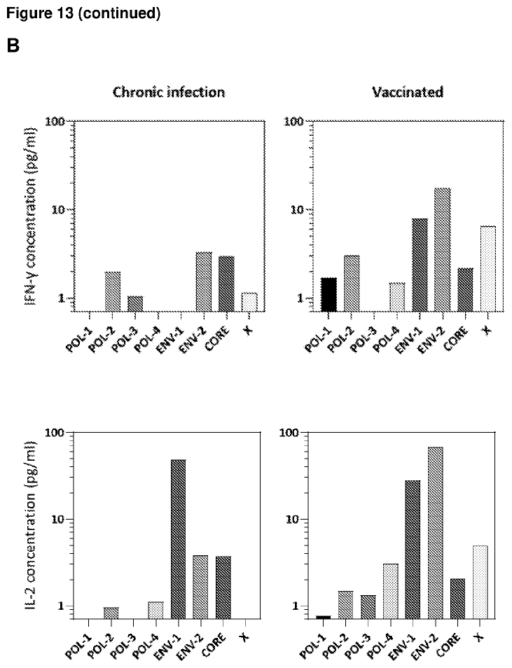

Eight HBV peptide pools of 15-mers (Tables 20-27) covering the proteome (Core, X, Envelope and Polymerase) of HBV (AB112063 (HBV Gen C)) were generated (FIG. 13A). The Core protein has 212 amino acids, so it requires 41 15-mer peptides overlapping by 10 amino acids to cover the whole protein, resulting in a single peptide pool; X has 154 amino acids, so it requires 29 15-mer peptides overlapping by 10 amino acids to cover the whole protein, resulting in a single peptide pool; Envelope has 389 amino acids, so it requires 76 15-mer peptides overlapping by 10 amino acids to cover the whole protein, resulting in a 2 peptide pools of about 40 peptides each; Polymerase has 843 amino acids, so it requires 167 15-mer peptides overlapping by 10 amino acids to cover the whole protein, resulting in 4 peptide pools of about 40 peptides each.

TABLE 20
Summary of Peptide Pool C (core).

| POOL C | | | |
|---|---|---|---|
| Peptide | | A.A | SEQ ID |
| Number | A.A Sequence | Position | Number |
| C_1 | MQLFHLCLIISCLRP | 1-15 | 480 |
| C_2 | LCLIISCLRPTVQAS | 6-20 | 481 |
| C_3 | SCLRPTVQASKLCLG | 11-25 | 482 |
| C_4 | TVQASKLCLGWLWGM | 16-30 | 483 |
| C_5 | KLCLGWLWGMDIDPY | 21-35 | 484 |
| C 6 | WLWGMDIDPYKEFGA | 26-40 | 485 |
| C_7 | DIDPYKEFGASVELL | 31-45 | 486 |
| C_8 | KEFGASVELLSFLPS | 36-50 | 487 |
| C_9 | SVELLSFLPSDFFPN | 41-55 | 488 |
| C_10 | SFLPSDFFPNIRDLL | 46-60 | 489 |
| C_11 | DFFPNIRDLLDTASA | 51-65 | 490 |
| C_12 | IRDLLDTASALFREA | 56-70 | 491 |
| C_13 | DTASALFREALESPE | 61-75 | 492 |
| C_14 | LFREALESPEHCTPH | 66-80 | 493 |
| C_15 | LESPEHCTPHHTAIR | 71-85 | 494 |
| C_16 | HCTPHHTAIRQAILC | 76-90 | 495 |
| C_17 | HTAIRQAILCWGELM | 81-95 | 496 |
| C_18 | QAILCWGELMNLATW | 86-100 | 497 |
| C_19 | WGELMNLATWVGSNL | 91-105 | 498 |
| C_20 | NLATWVGSNLEDPAS | 96-110 | 499 |
| C_21 | VGSNLEDPASRELVV | 101-115 | 500 |
| C_22 | EDPASRELVVGYVNV | 106-120 | 501 |
| C_23 | RELVVGYVNVNMGLK | 111-125 | 502 |
| C_24 | GYVNVNMGLKIRQLL | 116-130 | 503 |
| C_25 | NMGLKIRQLLWFHIS | 121-135 | 504 |
| C_26 | IRQLLWFHISCLTFG | 126-140 | 505 |
| C_27 | WFHISCLTFGRETVL | 131-145 | 506 |
| C_28 | CLTFGRETVLEYLVS | 136-150 | 507 |
| C_29 | RETVLEYLVSFGVWI | 141-155 | 508 |
| C_30 | EYLVSFGVWIRTPPA | 146-160 | 509 |
| C_31 | FGVWIRTPPAYRPPN | 151-165 | 510 |
| C_32 | RTPPAYRPPNAPILS | 156-170 | 511 |
| C_33 | YRPPNAPILSTLPET | 161-175 | 512 |
| C_34 | APILSTLPETTVVRR | 166-180 | 523 |
| C_35 | TLPETTVVRRRGRSP | 171-185 | 514 |
| C_36 | TVVRRRGRSPRRRTP | 176-190 | 515 |
| C_37 | RGRSPRRRTPSPRRR | 181-195 | 516 |
| C_38 | RRRTPSPRRRRSQSP | 186-200 | 517 |
| C_39 | SPRRRRSQSPRRRRS | 191-205 | 518 |
| C_40 | RSQSPRRRRSQSRGS | 196-210 | 519 |
| C_41 | RRRRSQSRGSQC | 201-215 | 520 |

TABLE 21
Summary of Peptide Pool X.

| POOL X | | | |
|---|---|---|---|
| Peptide | | A.A | SEQ ID |
| Number | A.A Sequence | Position | Number |
| X_1 | MAARLCCQLDPARDV | 1-15 | 521 |
| X_2 | CCQLDPARDVLCLRP | 6-20 | 522 |
| X_3 | PARDVLCLRPVGAES | 11-25 | 523 |
| X_4 | LCLRPVGAESRGRPV | 16-30 | 524 |
| X_5 | VGAESRGRPVSGAFG | 21-35 | 525 |
| X_6 | RGRPVSGAFGTLPSP | 26-40 | 526 |
| X_7 | SGAFGTLPSPSSSAV | 31-45 | 527 |
| X_8 | TLPSPSSSAVPTDHG | 36-50 | 528 |
| X_9 | SSSAVPTDHGAHLSL | 41-55 | 529 |
| X_10 | PTDHGAHLSLRGLPV | 46-60 | 530 |
| X_11 | AHLSLRGLPVCAFSS | 51-65 | 531 |
| X_12 | RGLPVCAFSSAGPCA | 56-70 | 532 |
| X_13 | CAFSSAGPCALRFTS | 61-75 | 533 |
| X_14 | AGPCALRFTSARRME | 66-80 | 534 |
| X_15 | LRFTSARRMETTVNA | 71-85 | 535 |
| X_16 | ARRMETTVNARQVLP | 76-90 | 536 |
| X_17 | TTVNARQVLPKVLHK | 81-95 | 537 |
| X_18 | RQVLPKVLHKRTLGL | 86-100 | 538 |
| X_19 | KVLHKRTLGLSAMST | 91-105 | 539 |
| X_20 | RTLGLSAMSTTDLEA | 96-110 | 540 |
| X_21 | SAMSTTDLEAYFKDR | 101-115 | 541 |
| X_22 | TDLEAYFKDRVFKDW | 106-120 | 542 |
| X_23 | YFKDRVFKDWEELGE | 111-125 | 543 |
| X_24 | VFKDWEELGEETRLM | 116-130 | 544 |
| X_25 | EELGEETRLMIFVLG | 121-135 | 545 |
| X_26 | ETRLMIFVLGGCRHK | 126-140 | 546 |
| X_27 | IFVLGGCRHKLVCSP | 131-145 | 547 |
| X_28 | GCRHKLVCSPAPCNF | 136-150 | 548 |
| X_29 | LVCSPAPCNFFTSA | 141-155 | 549 |

TABLE 22
Summary of Peptide Pool E1 (envelope).

| POOL E-1 | | | |
|---|---|---|---|
| Peptide | | A.A | SEQ ID |
| Number | A.A Sequence | Position | Number |
| E_1 | MGTNLSVPNPLGFFP | 1-15 | 550 |
| E_2 | SVPNPLGFFPDHQLD | 6-20 | 551 |
| E_3 | LGFFPDHQLDPAFGA | 11-25 | 552 |
| E_4 | DHQLDPAFGANSNNP | 16-30 | 553 |
| E_5 | PAFGANSNNPDWDFN | 21-35 | 554 |
| E_6 | NSNNPDWDFNPNKDQ | 26-40 | 555 |
| E_7 | DWDFNPNKDQWPAAN | 31-45 | 556 |
| E_8 | PNKDQWPAANQVGVG | 36-50 | 557 |
| E_9 | WPAANQVGVGSFGPG | 41-55 | 558 |
| E_10 | QVGVGSFGPGFTPPH | 46-60 | 559 |
| E_11 | SFGPGFTPPHGNLLG | 51-65 | 560 |
| E_12 | FTPPHGNLLGWSPQA | 56-70 | 561 |
| E_13 | GNLLGWSPQAQGILT | 61-75 | 562 |
| E_14 | WSPQAQGILTTVPAA | 66-80 | 563 |
| E_15 | QGILTTVPAAPPPAS | 71-85 | 564 |
| E_16 | TVPAAPPPASTNRQS | 76-90 | 565 |
| E_17 | PPPASTNRQSGRQPT | 81-95 | 566 |
| E_18 | TNRQSGRQPTPISLP | 86-100 | 567 |
| E_19 | GRQPTPISLPLRDSH | 91-105 | 568 |
| E_20 | PISLPLRDSHPQAMQ | 96-110 | 569 |
| E_21 | LRDSHPQAMQWNSST | 101-115 | 570 |
| E_22 | PQAMQWNSSTFHQAL | 106-120 | 571 |
| E_23 | WNSSTFHQALLDPKV | 111-125 | 572 |
| E_24 | FHQALLDPKVRGLYL | 116-130 | 573 |
| E_25 | LDPKVRGLYLPAGGS | 121-135 | 574 |
| E_26 | RGLYLPAGGSSSGTV | 126-140 | 575 |
| E_27 | PAGGSSSGTVNPVQT | 131-145 | 576 |
| E_28 | SSGTVNPVQTTASPI | 136-150 | 577 |
| E_29 | NPVQTTASPISSIFS | 141-155 | 578 |
| E_30 | TASPISSIFSRTGDP | 146-160 | 579 |
| E_31 | SSIFSRTGDPAPNME | 151-165 | 580 |
| E_32 | RTGDPAPNMESTTSG | 156-170 | 581 |
| E_33 | APNMESTTSGFLGPL | 161-175 | 582 |
| E_34 | STTSGFLGPLLVLQA | 166-180 | 583 |
| E_35 | FLGPLLVLQAGFFLL | 171-185 | 584 |
| E_36 | LVLQAGFFLLTRILT | 176-190 | 585 |
| E_37 | GFFLLTRILTIPQSL | 181-195 | 586 |
| E_38 | TRILTIPQSLDSWWT | 186-200 | 587 |

TABLE 23
Summary of Peptide Pool E2 (envelope).

| POOL E-2 | | | |
|---|---|---|---|
| Peptide | | A.A | SEQ ID |
| Number | A.A Sequence | Position | Number |
| E_39 | IPQSLDSWWTSLNFL | 191-205 | 588 |
| E_40 | DSWWTSLNFLGGAPT | 196-210 | 589 |
| E_41 | SLNFLGGAPTCSGQN | 201-215 | 590 |
| E_42 | GGAPTCSGQNLQSPT | 206-220 | 591 |
| E_43 | CSGQNLQSPTSNHSP | 211-225 | 592 |
| E_44 | LQSPTSNHSPTSCPP | 216-230 | 593 |
| E_45 | SNHSPTSCPPICPGY | 221-235 | 594 |
| E_46 | TSCPPICPGYRWMCL | 226-240 | 595 |
| E_47 | ICPGYRWMCLRRFII | 231-245 | 596 |
| E_48 | RWMCLRRFIIFLFIL | 236-250 | 597 |
| E_49 | RRFIIFLFILLLCLI | 241-255 | 598 |
| E_50 | FLFILLLCLIFLLVL | 246-260 | 599 |
| E_51 | LLCLIFLLVLLDYQG | 251-265 | 600 |
| E_52 | FLLVLLDYQGMLPVC | 256-270 | 601 |
| E_53 | LDYQGMLPVCPLLPG | 261-275 | 602 |
| E_54 | MLPVCPLLPGTSTTS | 266-280 | 603 |
| E_55 | PLLPGTSTTSMGPCK | 271-285 | 604 |
| E_56 | TSTTSMGPCKTCTTP | 276-290 | 605 |
| E_57 | MGPCKTCTTPAQGTS | 281-295 | 606 |
| E_58 | TCTTPAQGTSMFPSC | 286-300 | 607 |
| E_59 | AQGTSMFPSCCCTQP | 291-305 | 608 |
| E_60 | MFPSCCCTQPSDGNC | 296-310 | 609 |
| E_61 | CCTQPSDGNCTCIPI | 301-315 | 610 |
| E_62 | SDGNCTCIPIPSSWA | 306-320 | 611 |
| E_63 | TCIPIPSSWAFARFL | 311-325 | 612 |
| E_64 | PSSWAFARFLWEWAS | 316-330 | 613 |
| E_65 | FARFLWEWASVRFSW | 321-335 | 614 |
| E_66 | WEWASVRFSWLSLLV | 326-340 | 615 |
| E_67 | VRFSWLSLLVPFVQW | 331-345 | 616 |
| E_68 | LSLLVPFVQWFVGLS | 336-350 | 617 |
| E_69 | PFVQWFVGLSPTVWL | 341-355 | 618 |
| E_70 | FVGLSPTVWLSVIWM | 346-360 | 619 |
| E_71 | PTVWLSVIWMMWYWG | 351-365 | 620 |
| E_72 | SVIWMMWYWGRSLYN | 356-370 | 621 |
| E_73 | MWYWGRSLYNILNPF | 361-375 | 622 |
| E_74 | RSLYNILNPFLPLLP | 366-380 | 623 |
| E_75 | ILNPFLPLLPIFFCL | 371-385 | 624 |
| E_76 | LPLLPIFFCLWVYI | 386-390 | 625 |

TABLE 24
Summary of Peptide Pool Pol-1 (Polymerase).

| POOL | | | |
|---|---|---|---|
| Pol-1 | | | |
| Peptide | | A.A | SEQ ID |
| Number | A.A Sequence | Position | Number |
| Pol_1 | MPLSYQHFRKLLLLD | 1-15 | 626 |
| Pol_2 | QHFRKLLLLDDEAGP | 6-20 | 627 |
| Pol_3 | LLLLDDEAGPLEEEL | 11-25 | 628 |
| Pol_4 | DEAGPLEEELPRLAD | 16-30 | 629 |
| Pol_5 | LEEELPRLADEGLNH | 21-35 | 630 |
| Pol_6 | PRLADEGLNHRVAED | 26-40 | 631 |
| Pol_7 | EGLNHRVAEDLNLGD | 31-45 | 632 |
| Pol_8 | RVAEDLNLGDLNVSI | 36-50 | 633 |
| Pol_9 | LNLGDLNVSIPWTHK | 41-55 | 634 |
| Pol_10 | LNVSIPWTHKVGNFT | 46-60 | 635 |
| Pol_11 | PWTHKVGNFTGLYSS | 51-65 | 636 |
| Pol_12 | VGNFTGLYSSTVPVF | 56-70 | 637 |
| Pol_13 | GLYSSTVPVFNPEWK | 61-75 | 638 |
| Pol_14 | TVPVFNPEWKTPSFP | 66-80 | 639 |
| Pol_15 | NPEWKTPSFPNIHLK | 71-85 | 640 |
| Pol_16 | TPSFPNIHLKEDIIN | 76-90 | 641 |
| Pol_17 | NIHLKEDIINRCQQY | 81-95 | 642 |
| Pol_18 | EDIINRCQQYVGPLT | 86-100 | 643 |
| Pol_19 | RCQQYVGPLTVNEKR | 91-105 | 644 |
| Pol_20 | VGPLTVNEKRRLKVT | 96-110 | 645 |
| Pol_21 | VNEKRRLKVTMPARF | 101-115 | 646 |
| Pol_22 | RLKVTMPARFYPNLT | 106-120 | 647 |
| Pol_23 | MPARFYPNLTKYLPL | 111-125 | 648 |
| Pol_24 | YPNLTKYLPLDKGIK | 116-130 | 649 |
| Pol_25 | KYLPLDKGIKPYYPE | 121-135 | 650 |
| Pol_26 | DKGIKPYYPEHIVNH | 126-140 | 651 |
| Pol_27 | PYYPEHIVNHYFQTR | 131-145 | 652 |
| Pol_28 | HIVNHYFQTRHYLHT | 136-150 | 653 |
| Pol_29 | YFQTRHYLHTLWKAG | 141-155 | 654 |
| Pol_30 | HYLHTLWKAGILYKR | 146-160 | 655 |
| Pol_31 | LWKAGILYKRETTRS | 151-165 | 656 |
| Pol_32 | ILYKRETTRSASFCG | 156-170 | 657 |
| Pol_33 | ETTRSASFCGSPYSW | 161-175 | 658 |
| Pol_34 | ASFCGSPYSWEQELQ | 166-180 | 659 |
| Pol_35 | SPYSWEQELQHGTLV | 171-185 | 660 |
| Pol_36 | EQELQHGTLVFQTST | 176-190 | 661 |
| Pol_37 | HGTLVFQTSTRHGDE | 181-195 | 662 |
| Pol_38 | FQTSTRHGDESFGSQ | 186-200 | 663 |
| Pol_39 | RHGDESFGSQSSGIL | 191-205 | 664 |
| Pol_40 | SFGSQSSGILSRSPV | 196-210 | 665 |
| Pol_41 | SSGILSRSPVGPGIR | 201-215 | 666 |
| Pol_42 | SRSPVGPGIRSQFKQ | 206-220 | 667 |

TABLE 25
Summary of Peptide Pool Pol-2 (Polymerase).

| POOL | | | |
|---|---|---|---|
| Pol-2 | | | |
| Peptide | | A.A | SEQ ID |
| Number | A.A Sequence | Position | Number |
| Pol_43 | GPGIRSQFKQSRLGL | 211-225 | 668 |
| Pol_44 | SQFKQSRLGLQPQQG | 216-230 | 669 |
| Pol_45 | SRLGLQPQQGSMASG | 221-235 | 670 |
| Pol_46 | QPQQGSMASGKPGRS | 226-240 | 671 |
| Pol_47 | SMASGKPGRSGIIRA | 231-245 | 672 |
| Pol_48 | KPGRSGIIRARVHPT | 236-250 | 673 |
| Pol_49 | GIIRARVHPTTRQSF | 241-255 | 674 |
| Pol_50 | RVHPTTRQSFGVEPA | 246-260 | 675 |
| Pol_51 | TRQSFGVEPAGSGHI | 251-265 | 676 |
| Pol_52 | GVEPAGSGHIDNSTS | 256-270 | 677 |
| Pol_53 | GSGHIDNSTSSASSC | 261-275 | 678 |
| Pol_54 | DNSTSSASSCLHQSA | 266-280 | 679 |
| Pol_55 | SASSCLHQSAVRKTA | 271-285 | 680 |
| Pol_56 | LHQSAVRKTAYSHLS | 276-290 | 681 |
| Pol_57 | VRKTAYSHLSTSKRQ | 281-295 | 682 |
| Pol_58 | YSHLSTSKRQSSSGH | 286-300 | 683 |
| Pol_59 | TSKRQSSSGHAVELQ | 291-305 | 684 |
| Pol_60 | SSSGHAVELQHIPPS | 296-310 | 685 |
| Pol_61 | AVELQHIPPSSARSQ | 301-315 | 686 |
| Pol_62 | HIPPSSARSQSEGPI | 306-320 | 687 |
| Pol_63 | SARSQSEGPIPSCWW | 311-325 | 688 |
| Pol_64 | SEGPIPSCWWLQFRN | 316-330 | 689 |
| Pol_65 | PSCWWLQFRNSKPCS | 321-335 | 690 |
| Pol_66 | LQFRNSKPCSDYCLS | 326-340 | 691 |
| Pol_67 | SKPCSDYCLSHIVNL | 331-345 | 692 |
| Pol_68 | DYCLSHIVNLLEDWG | 336-350 | 693 |
| Pol_69 | HIVNLLEDWGPCTEY | 341-355 | 694 |
| Pol_70 | LEDWGPCTEYGEHHI | 346-360 | 695 |
| Pol_71 | PCTEYGEHHIRIPRT | 351-365 | 696 |
| Pol_72 | GEHHIRIPRTPARVT | 356-370 | 697 |
| Pol_73 | RIPRTPARVTGGVFL | 361-375 | 698 |
| Pol_74 | PARVTGGVFLVDKNP | 366-380 | 799 |
| Pol_75 | GGVFLVDKNPHNTTE | 371-385 | 700 |
| Pol_76 | VDKNPHNTTESRLVV | 386-390 | 701 |
| Pol_77 | HNTTESRLVVDFSQF | 381-395 | 702 |
| Pol_78 | SRLVVDFSQFSRGST | 386-400 | 703 |
| Pol_79 | DFSQFSRGSTHVFWP | 391-405 | 704 |
| Pol_80 | SRGSTHVFWPKFAVP | 396-410 | 705 |
| Pol_81 | HVFWPKFAVPNLQSL | 401-415 | 706 |
| Pol_82 | KFAVPNLQSLTNLLS | 406-419 | 707 |
| Pol_83 | NLQSLTNLLSSNLSW | 411-425 | 708 |
| Pol_84 | TNLLSSNLSWLSLDV | 416-430 | 709 |

TABLE 26
Summary of Peptide Pool Pol-3 (Polymerase).

| POOL | | | |
|---|---|---|---|
| Pol-3 | | | |
| Peptide | | A.A | SEQ ID |
| Number | A.A Sequence | Position | Number |
| Pol_85 | SNLSWLSLDVSAAFY | 421-435 | 710 |
| Pol_86 | LSLDVSAAFYHIPLH | 426-440 | 711 |
| Pol_87 | SAAFYHIPLHPAAMP | 431-445 | 712 |
| Pol_88 | HIPLHPAAMPHLLVG | 436-450 | 713 |
| Pol_89 | PAAMPHLLVGSSGLP | 441-455 | 714 |
| Pol_90 | HLLVGSSGLPRYVAR | 446-460 | 715 |
| Pol_91 | SSGLPRYVARLSSTS | 451-465 | 716 |
| Pol_92 | RYVARLSSTSRNINY | 456-470 | 717 |
| Pol_93 | LSSTSRNINYQHGTM | 461-475 | 718 |
| Pol_94 | RNINYQHGTMQDLHD | 466-480 | 719 |
| Pol_95 | QHGTMQDLHDTCSRN | 471-485 | 720 |
| Pol_96 | QDLHDTCSRNLYVSL | 476-490 | 721 |
| Pol_97 | TCSRNLYVSLLLLYT | 481-495 | 722 |
| Pol_98 | LYVSLLLLYTTFGRK | 486-500 | 723 |
| Pol_99 | LLLYTTFGRKLHLYS | 491-505 | 724 |
| Pol_100 | TFGRKLHLYSHPIIL | 496-510 | 725 |
| Pol_101 | LHLYSHPIILGFRKI | 501-515 | 726 |
| Pol_102 | HPIILGFRKIPMGVG | 506-520 | 727 |
| Pol_103 | GFRKIPMGVGLSPFL | 511-525 | 728 |
| Pol_104 | PMGVGLSPFLLAQFT | 516-530 | 729 |
| Pol_105 | LSPFLLAQFTSAICS | 521-535 | 730 |
| Pol_106 | LAQFTSAICSVVRRA | 526-540 | 731 |
| Pol_107 | SAICSVVRRAFPHCL | 531-545 | 732 |
| Pol_108 | VVRRAFPHCLAFSYM | 536-550 | 733 |
| Pol_109 | FPHCLAFSYMDDVVL | 541-555 | 734 |
| Pol_110 | AFSYMDDVVLGAKSV | 546-560 | 735 |
| Pol_111 | DDVVLGAKSVQHLES | 551-565 | 736 |
| Pol_112 | GAKSVQHLESLFTAV | 556-570 | 737 |
| Pol_113 | QHLESLFTAVTNFLL | 561-575 | 738 |
| Pol_114 | LFTAVTNFLLSLGVH | 566-580 | 739 |
| Pol_115 | TNFLLSLGVHLNPTK | 571-585 | 740 |
| Pol_116 | SLGVHLNPTKTKRWG | 576-590 | 741 |
| Pol_117 | LNPTKTKRWGYSLNF | 581-595 | 742 |
| Pol_118 | TKRWGYSLNFMGYVI | 586-600 | 743 |
| Pol_119 | YSLNFMGYVIGSWGT | 591-605 | 744 |
| Pol_120 | MGYVIGSWGTLPQEH | 596-610 | 745 |
| Pol_121 | GSWGTLPQEHIVHKI | 601-615 | 746 |
| Pol_122 | LPQEHIVHKIKQCFR | 606-620 | 747 |
| Pol_123 | IVHKIKQCFRKLPLN | 611-625 | 748 |
| Pol_124 | KQCFRKLPLNRPIDW | 616-630 | 749 |
| Pol_125 | KLPLNRPIDWKVCQR | 621-635 | 750 |
| Pol_126 | RPIDWKVCQRIVGLL | 626-640 | 751 |

TABLE 27
Summary of Peptide Pool Pol-4 (Polymerase).

| POOL | | | |
|---|---|---|---|
| Pol-4 | | | |
| Peptide | | A.A | SEQ ID |
| Number | A.A Sequence | Position | Number |
| Pol_127 | KVCQRIVGLLGFAAP | 631-645 | 752 |
| Pol_128 | IVGLLGFAAPFTQCG | 636-650 | 753 |
| Pol_129 | GFAAPFTQCGYPALM | 641-655 | 754 |
| Pol_130 | FTQCGYPALMPLSAC | 646-660 | 755 |
| Pol_131 | YPALMPLSACIQAKR | 651-665 | 756 |
| Pol_132 | PLSACIQAKRAFTFS | 656-670 | 757 |
| Pol_133 | IQAKRAFTFSPTYRA | 661-675 | 758 |
| Pol_134 | AFTFSPTYRAFLCKQ | 666-680 | 759 |
| Pol_135 | PTYRAFLCKQYMNLY | 671-685 | 760 |
| Pol_136 | FLCKQYMNLYPVARQ | 676-690 | 761 |
| Pol_137 | YMNLYPVARQRPGLC | 681-695 | 762 |
| Pol_138 | PVARQRPGLCQVFAD | 686-700 | 763 |
| Pol_139 | RPGLCQVFADATPTG | 691-705 | 764 |
| Pol_140 | QVFADATPTGWGLAI | 696-710 | 765 |
| Pol_141 | ATPTGWGLAIGHQRM | 701-715 | 766 |
| Pol_142 | WGLAIGHQRMRGTFV | 706-720 | 767 |
| Pol_143 | GHQRMRGTFVAPLPI | 711-725 | 768 |
| Pol_144 | RGTFVAPLPIHTAEL | 716-730 | 769 |
| Pol_145 | APLPIHTAELLAACF | 721-735 | 770 |
| Pol_146 | HTAELLAACFARSRS | 726-740 | 771 |
| Pol_147 | LAACFARSRSGAKLI | 731-745 | 772 |
| Pol_148 | ARSRSGAKLIGTDNS | 736-750 | 773 |
| Pol_149 | GAKLIGTDNSVVLSR | 741-755 | 774 |
| Pol_150 | GTDNSVVLSRKYTSF | 746-760 | 775 |
| Pol_151 | VVLSRKYTSFPWLLG | 751-765 | 776 |
| Pol_152 | KYTSFPWLLGCAANW | 756-770 | 777 |
| Pol_153 | PWLLGCAANWILRGT | 761-775 | 778 |
| Pol_154 | CAANWILRGTSFVYV | 766-780 | 779 |
| Pol_155 | ILRGTSFVYVPSALN | 771-785 | 780 |
| Pol_156 | SFVYVPSALNPADDP | 776-790 | 781 |
| Pol_157 | PSALNPADDPSRGRL | 781-795 | 782 |
| Pol_158 | PADDPSRGRLGLYRP | 786-800 | 783 |
| Pol_159 | SRGRLGLYRPLLRLP | 791-805 | 784 |
| Pol_160 | GLYRPLLRLPFRPTT | 796-810 | 785 |
| Pol_161 | LLRLPFRPTTGRTSL | 801-815 | 786 |
| Pol_162 | FRPTTGRTSLYAVSP | 806-820 | 787 |
| Pol_163 | GRTSLYAVSPSVPSH | 811-825 | 788 |
| Pol_164 | YAVSPSVPSHLPDRV | 816-830 | 789 |
| Pol_165 | SVPSHLPDRVHFASP | 821-835 | 790 |
| Pol_166 | LPDRVHFASPLHVAW | 826-840 | 791 |
| Pol_167 | HFASPLHVAWRPP | 831-843 | 792 |

### Human Samples

Whole blood was isolated from either a patient with chronic HBV infection or a healthy individual who was vaccinated for HBV (with recombinant HBV envelope vaccine) and tested within 24 hours after blood draw (FIG. 13B). 400 μl aliquots were separately mixed with 100 μl RPMI containing each of the HBV peptide pools (2 μg/ml final concentration per peptide) or a DMSO control and incubated for a period extending overnight, to allow activation of responsive T cells. A plasma fraction was isolated from each of the incubated samples and the level of cytokines in the sample measured using an Ella™ multi-analyte ELISA machine (ProteinSimple, CA, USA).

It would be understood that the parameters described in the example may be changed and still achieve a valid assay. For example, the ratio of blood to RMPI media can range from 100% blood to 50% blood; 100 μl aliquots or more than 400 μl might be used, but a larger blood sample would be required to test multiple peptide pools. RPMI may be exchanged with other cell culture media. The final concentration of peptides in an assay mixture may be from 1 to 5 μg/ml. The control sample may contain DMSO or may be an unstimulated blood sample. The incubation period may range between about 6-24 h.

HBV reactive T cells in infected individuals can be detected by quantifying the amount of secreted cytokines after the direct addition of the peptide pools into whole blood. At the moment, we have selected the detection of both IFN-γ and IL-2, two cytokines commonly secreted by T cells, as a means of detection.

From the data (FIG. 13B), we observed that T cells specific for the HBV envelope protein were readily detected in the chronic HBV patient and the vaccinated individual. The secretion of IL-2 appears to be more sensitive than IFN-γ to detect the HBV-specific T cells. In conclusion, we demonstrate that the whole blood assay can be readily applied for the detection of T cells specific for other viruses by utilising overlapping peptides specific for the virus of interest.

### SUMMARY

The assays presented here are based on the ability of SARS-CoV-2 T cells to respond to different peptides covering different proteins of the virus. With the possibility to use different peptides pools, our approach represents a flexible strategy that can be easily utilized to detect the presence of T cells responding to emerging mutant strains and, thus, immediately gauge the impact that viral mutation might have on cellular immunity. Moreover, the methods exemplified herein are applicable to viruses other than SARS-CoV-2 and its variants.

Hence, a diagnostic method that can be easily adapted to detect the degree of cellular immunity is an urgently needed complement to the currently available tests measuring viral presence or antibody titers.

### REFERENCES

1. Braun, J., Loyal, L., Frentsch, M., Wendisch, D., Georg, P., Kurth, F., Hippenstiel, S., Dingeldey, M., Kruse, B., Fauchere, F., Baysal, E., Mangold, M., Henze, L., Lauster, R., Mall, M., Beyer, K., Roehmel, J., Schmitz, J., Miltenyi, S., Mueller, M. A., Witzenrath, M., Suttorp, N., Kern, F., Reimer, U., Wenschuh, H., Drosten, C., Corman, V. M., Giesecke-Thiel, C., Sander, L.-E., Thiel, A., 2020. Presence of SARS-CoV-2 reactive T cells in COVID-19 patients and healthy donors. medRxiv 1-12. doi:10.1101/2020.04.17.20061440.
2. Camara C., et al., Differential effects of the second SARS-CoV-2 mRNA vaccine dose on T cell immunity in naïve and COVID-19 recovered individuals. bioRxiv 2021.03.22.436441; doi: https://doi.org/10.1101/2021.03.22.436441.
3. Dong, T., Peng, Y., Mentzer, A. J., Liu, G., Yao, X., Yin, Z., Dong, D., Dejnirattisai, W., Turtle, L., Rostron, T., Subramaniam, K., Thomson, P., Zhang, P., Dold, C., Ratcliff, J., de Silva, T., Sopp, P., Wellington, D., Rajapaksa, U., Paes, W., Borrow, P., Kessler, B. M., Fry, J. W., Schwabe, N. F., Semple, M. G., Baillie, J. K., Openshaw, P. J., Cornall, R. J., Conlon, C., Screaton, G., Klenerman, P., Mongkolsapaya, J., McMichael, A., Knight, J. C., Ogg, G., Simmonds, P., Lockett, T., Levin, R., Moore, S. C., Salio, M., Napolitani, G., Chen, Y.-L., Dunachie, S., Supasa, P., Liu, C., Lopez-Camacho, C., Slon-Campos, J., Zhao, Y., Stuart, D. I., Paeson, G., Grimes, J., Antson, F., Bayfield, O. W., Hawkins, D. E., Ker, D.-S., Ansari, A., Barnes, E., Frater, J., Kerr, G., Goulder, P., 2020. Broad and strong memory CD4+ and CD8+ T cells induced by SARS-CoV-2 in UK convalescent COVID-19 patients. bioRxiv 1-36. doi:10.1101/2020.06.05.134551.
4. Grifoni A, Sidney J, Zhang Y, Scheuermann R H, Peters B, Sette A. A Sequence Homology and Bioinformatic Approach Can Predict Candidate Targets for Immune Responses to SARS-CoV-2. Cell Host Microbe 2020; 27(4): 671-680.e2. doi:10.1016/j.chom.2020.03.002.
5. Grifoni, A., n.d. Targets of T cell responses to SARS-CoV-2 coronavirus in humans with COVID-19 disease and unexposed individuals. Cell. doi:10.1016/j.cell.2020.05.015.
6. Ichikawa, A. et al. CXCL10-CXCR3 enhances the development of neutrophil-mediated fulminant lung injury of viral and nonviral origin. Am J Respir Crit Care Med 187, 65-77 (2013).
7. Kalimuddin S, Tham C Y L, Qui M, de Alwis R, Sim J X Y, Lim J M E, Tan H C, Syenina A, Zhang S L, Le Bert N, Tan A T, Leong Y S, Yee J X, Ong E Z, Ooi E E, Bertoletti A, Low J G. Early T cell and binding antibody responses are associated with COVID-19 RNA vaccine efficacy onset. Med (N Y). 2021 Jun. 11; 2(6):682-688.e4. doi: 10.1016/j.medj.2021.04.003.
8. Le Bert N, Tan A T, Kunasegaran K, et al. SARS-CoV-2-specific T cell immunity in cases of COVID-19 and SARS, and uninfected controls. Nature 2020; 584(7821): 457-62.
9. Le Bert, N. et al. Highly functional virus-specific cellular immune response in asymptomatic SARS-CoV-2 infection. J Exp Med 218(5):e20202617 (2021).
10. Long, Q.-X., Liu, B.-Z., Deng, H.-J., Wu, G.-C., Deng, K., Chen, Y.-K., Liao, P., Qiu, J.-F., Lin, Y., Cai, X.-F., Wang, D.-Q., Hu, Y., Ren, J.-H., Tang, N., Xu, Y.-Y., Yu, L.-H., Mo, Z., Gong, F., Zhang, X.-L., Tian, W.-G., Hu, L., Zhang, X.-X., Xiang, J.-L., Du, H.-X., Liu, H.-W., Lang, C.-H., Luo, X.-H., Wu, S.-B., Cui, X.-P., Zhou, Z., Zhu, M.-M., Wang, J., Xue, C.-J., Li, X.-F., Wang, L., Li, Z.-J., Wang, K., Niu, C.-C., Yang, Q.-J., Tang, X.-J., Zhang, Y., Liu, X.-M., Li, J.-J., Zhang, D.-C., Zhang, F., Liu, P., Yuan, J., Li, Q., Hu, J.-L., Chen, J., Huang, A.-L., 2020. Antibody responses to SARS-CoV-2 in patients with COVID-19. Nat Med 1-15. doi:10.1038/s41591-020-0897-1.
11. Luster, A. D., Unkeless, J. C. & Ravetch, J. V. Gamma-interferon transcriptionally regulates an early-response gene containing homology to platelet proteins. Nature 315, 672-676 (1985).
12. Tauzin, A. et al., A single BNT162b2 mRNA dose elicits antibodies with Fc-mediated effector functions and boost pre-existing humoral and T cell responses. bioRxiv. 2021 Mar. 18; 2021.03.18.435972. doi: 10.1101/2021.03.18.435972.
13. Zhao, J., Alshukairi, A. N., Baharoon, S. A., Ahmed, W. A., Bokhari, A. A., Nehdi, A. M., Layqah, L. A., Alghamdi, M. G., Gethamy, Al, M. M., Dada, A. M., Khalid, I., Boujelal, M., Johani, Al, S. M., Vogel, L., Subbarao, K., Mangalam, A., Wu, C., Eyck, Ten, P., Perlman, S., Zhao, J., 2017. Recovery from the Middle East respiratory syndrome is associated with antibody and T-cell responses. Science Immunology 2, eaan5393. doi:10.1126/sciimmunol.aan5393.

1. An in vitro method of discriminating past or currently virus-infected subjects from virus un-infected subjects, comprising:
assaying a sample comprising whole blood, or comprising or derived from bronchoalveolar lavage (BAL) fluid, nasal swabs, or nasopharyngeal aspirate from a subject to determine whether it comprises T cells reactive to one or more virus peptide pools, wherein said peptide pools are separately derived from virus antigenic structural and non-structural proteins, wherein;
(a) if the sample T cells are reactive to a majority of the peptide pools derived from the virus antigenic proteins, in comparison to unstimulated or DMSO treated cells, the subject is identified as past or currently infected by the virus, or
(b) if the sample T cells are reactive to 0, or a minority of the peptide pools derived from the virus antigenic proteins, in comparison to unstimulated or DMSO treated cells,
the subject is identified as having been uninfected by the virus.

2. The method of claim 1, wherein the virus is:
a) a coronavirus, selected from the group comprising MERS-CoV, SARS-CoV, SARS-CoV-2, HKU1, OC43, NL63 and 229E or variant thereof; or
b) hepatitis B virus (HBV) or variant thereof.

3. The method of claim 2, wherein:
a) the virus antigenic structural and non-structural proteins are membrane (M), nucleoprotein (NP) and/or Spike (S) proteins; or
b) the virus antigenic structural and non-structural proteins are Polymerase (Pol), Envelope (E), Core (C) and/or X proteins.

4. The method of claim 3, wherein the virus is a) SARS-CoV-2 or b) HBV; and
ai) an M peptide pool comprises or consists of at least one peptide derived from an M protein comprising the amino acid sequence set forth in SEQ ID NO: 793;
ii) an NP peptide pool comprises or consists of at least one peptide derived from an NP protein comprising the amino acid sequence set forth in SEQ ID NO: 794; and
iii) an S peptide pool comprises or consists of at least one peptide derived from an S protein comprising the amino acid sequence set forth in SEQ ID NO: 795; or
bi) a Pol peptide pool comprises or consists of at least one peptide derived from an Pol protein comprising the amino acid sequence set forth in SEQ ID NO: 796;
ii) an E peptide pool comprises or consists of at least one peptide derived from an E protein comprising the amino acid sequence set forth in SEQ ID NO: 797;
iii) a C peptide pool comprises or consists of at least one peptide derived from a C protein comprising the amino acid sequence set forth in SEQ ID NO: 798; and
iv) an X peptide pool comprises or consists of at least one peptide derived from an X protein comprising the amino acid sequence set forth in SEQ ID NO: 799.

5. The method of claim 4, wherein;
ai) an M peptide pool comprises or consists of at least one peptide selected from peptides having the amino acid sequences set forth in SEQ ID Nos: 1-43,
ii) an NP peptide pool comprises or consists of at least one peptide selected from peptides having the amino acid sequences set forth in SEQ ID Nos: 44-125, and
iii) an S peptide pool comprises or consists of at least one peptide selected from peptides having the amino acid sequences set forth in SEQ ID Nos: 126-180; or
bi) a Pol peptide pool comprises or consists of at least one peptide selected from peptides having the amino acid sequences set forth in SEQ ID Nos: 626-792,
ii) an E peptide pool comprises or consists of at least one peptide selected from peptides having the amino acid sequences set forth in SEQ ID Nos: 550-625;
iii) a C peptide pool comprises or consists of at least one peptide selected from peptides having the amino acid sequences set forth in SEQ ID Nos: 480-520; and
iv) an X peptide pool comprises or consists of at least one peptide selected from peptides having the amino acid sequences set forth in SEQ ID Nos: 521-549.

6. The method of claim 5, wherein the NP peptide pool is divided into 2 pools, NP1 and NP2; or
wherein the Pol peptide pool and/or the E peptide pool is/are divided into a plurality of pools.

7. The method of claim 6, wherein;
(a) if the sample T cells are reactive to 3 or 4 of the peptide pools derived from M, NP1, NP2 and S, in comparison to unstimulated or DMSO treated cells, the subject is identified as past or currently infected by SARS-CoV-2,
(b) if the sample T cells are reactive to 0, 1 or 2 of the peptide pools derived from M, NP1, NP2 and S, in comparison to unstimulated or DMSO treated cells, the subject is identified as having been uninfected by SARS-CoV-2.

8. The method of claim 1, comprising the steps of:
a) mixing the sample with each of said peptide pools to produce:
i) assay samples corresponding to M, NP and S; or
ii) E, Pol, C and X;
b) incubating each mixture for a period to allow for T cell activation;
c) measuring the level of at least one secreted cytokine in each said mixture and determining whether the level of at least one secreted cytokine is above a threshold control value to indicate a positive T-cell reaction; and
d) counting the number of peptide pools that are positive.

9. The method of claim 6, comprising the steps of:
a) mixing the sample with each of said peptide pools to produce:
i) 4 assay samples corresponding to M, NP1, NP2 and S; or
ii) 8 assay samples corresponding to C, Pol1, Pol2, Pol3, Pol4, E1, E2 and X;
b) incubating each mixture for a period to allow for T cell activation;
c) measuring the level of at least one secreted cytokine in each said mixture and determining whether the level of at least one secreted cytokine is above a threshold control value to indicate a positive T-cell reaction; and
d) counting the number of peptide pools that are positive.

10. The method of claim 1, wherein if the sample T cells are reactive to 50% or more of the coronavirus peptide pools, in comparison to unstimulated or DMSO-treated cells, the subject is identified as past or currently infected by coronavirus.

11. An in vitro method of determining whether a vaccine or previously virus-infected subject has T cells whose activation may be reduced by a virus variant, such as a variant of concern (VOC), comprising:
assaying a sample comprising whole blood, or comprising or derived from bronchoalveolar lavage (BAL fluid), nasal swabs, or nasopharyngeal aspirate from a subject to determine whether it comprises T cells reactive to one or more virus peptide pools, wherein said peptide pools are separately derived from (A) the whole virus antigenic protein present in the vaccine or corresponding to an antigenic protein from the virus that infected the subject, (B) non-conserved regions of said virus antigenic protein that are mutated in the virus variant, and (C) virus variant mutated non-conserved regions of the vaccine antigenic protein or corresponding to an antigenic protein from the virus that infected the subject, wherein;
the number or proportion of reactive T cells present in each pool is analyzed and utilized to derive in each single individual, the frequency of T cells directed towards the whole virus antigenic protein (PBMC stimulated with peptide pool A), the frequency of T cells directed toward non-conserved regions of said virus antigenic protein that are mutated in the virus variant (PBMC stimulated with Pool B) and the frequency of T cells inhibited by amino acid mutations present in virus variant mutated non-conserved regions (PBMC stimulated with pool C), wherein;
(a) if the sample T cells are reactive to peptide pool A, the subject has T cells responsive against the virus antigenic protein, and
(b) if the sample T cells are similarly reactive to pool B and pool C, the impact of the amino acid mutations in the variant are negligible on the total T cell response against the said virus antigenic protein;
(c) if the sample T cells reacts differently to pool B and pool C, the impact of the amino acid mutations in the variant on the total T cell response against the said virus antigenic protein can be estimated by the proportion of pool C against pool B response,
wherein the method provides an estimation of the ability of T cells of the subject to recognize the conserved and non-conserved region of different vaccine antigenic proteins or virus that infected the subject, and of the ability of mutations to reduce the T cell response towards variants.

12. The method of claim 11, wherein:
i) the virus is a coronavirus, selected from the group comprising MERS-CoV, SARS-CoV, SARS-CoV-2, KHU1, OC43, NL63 and 229E; or
ii) the virus is HBV or variants thereof; and/or
iii) the virus antigenic protein is an M, NP, or S protein; or an E, Pol, C or X protein; and/or
iv) peptide pool A and pool B are derived from a wild-type virus.

13.-14. (canceled)

15. The method of claim 12 or, wherein iii) the virus antigenic protein is an M, NP, or S protein; or an E, Pol, C or X protein; and/or iv) peptide pool A and pool B are derived from a wild-type virus; and
wherein:
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C peptide protein comprises the amino acid sequence set forth in SEQ ID NO: 798; and
iv) the X protein comprises the amino acid sequence set forth in SEQ ID NO: 799.

16. The method of claim 12, wherein iv) peptide pool A and pool B are derived from a wild-type virus; and wherein the wildtype virus is SARS-CoV-2 wildtype and the variant is selected from the group comprising B.1.617.2 (Delta), B.1.1.7 (Alpha V1), B.1.351 (Beta V2), P.1 (Gamma, V3), B.1.617.1 (Kappa), (P2), B.1.427/9 (Epsilon), B.1.525 (Eta), B.1.526 (Iota), C.37 (Lambda), B.1.621 and B.1.620; or the virus is HBV C.

17. The method of claim 11, comprising the steps of:
a) mixing the sample with each of said peptide pools A, B, and C to produce assay samples;
b) incubating each mixture formed for a period to allow T cell activation;
c) measuring the level of at least one secreted cytokine in each said mixture and determining whether the level of at least one secreted cytokine is above a threshold control value to indicate a positive T-cell reaction; and
d) determining the number or proportion of reactive T cells present in each pool.

18. The method of any one of claim 8, wherein the secreted cytokine is selected from the group comprising IFN-gamma, IL-2, CXCL9, CXCL10, TNF-alpha, IL-6, IL-10 and IL-1.

19.-20. (canceled)

21. The method of claim 1, wherein:
a) whole blood is mixed with each of said peptide pools;
b)i) each mixture is incubated for at least 6 h;
b)ii) a plasma fraction of the mixture is isolated;
c) the level of at least one secreted cytokine in each said plasma fraction is measured and compared to a threshold control value to indicate a positive or negative T cell reaction.

22. A method to quantify the presence of virus-specific T cells in a biological sample comprising or derived from blood, broncholavage (BAL fluid), nasal swabs, or nasopharyngeal aspirate from a subject, comprising;
a) Mixing the biological sample with one or more virus peptide pools, wherein said peptide pools are separately derived from virus antigenic structural or non-structural proteins;
b) incubating the mixture formed for a period to allow T cell activation;
c) Rupture the cells from b);
d) Aliquot a sample from c) into PCR reagents, ACTIN (or other internal control) forward and reverse primers, ACTIN (or other internal control) probe, CXCL10 forward and reverse primers and CXCL10 probe for dqPCR; and/or
e) extract RNA from a sample from c) and add a portion into PCR reagents, ACTIN (or other internal control) forward and reverse primers, ACTIN (or other internal control) probe, CXCL10 forward and reverse primers and CXCL10 probe for qPCR;
f) perform cycles of dqPCR and/or qPCR for d) and e), respectively; and
g) quantitate the expression of CXCL10 in the sample and compare with a control,
wherein an elevated CXCL10 level indicates the presence of virus-specific T cells in the subject sample.

23. The method of claim 22, wherein the virus is a coronavirus, selected from the group comprising MERS-CoV, SARS-CoV, SARS-CoV-2, KHU1, OC43, NL63 and 229E or variant mutants thereof; or HBV or variant mutant thereof: and/or wherein:
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C protein comprises the amino acid sequence set forth in SEQ ID NO: 798; and
iv) the X protein comprises the amino acid sequence set forth in SEQ ID NO: 799;
and/or
wherein the peptide pools comprise one or more M, NP and S peptides listed in Tables 1-4 and 7-19; or one or more Pol, E, C and X peptides listed in Tables 20-27.

24.-25. (canceled)

26. A method of treatment or prophylaxis, comprising administering, respectively, to a subject with T cells reactive to:
i) a majority of peptide pools derived from virus antigenic proteins, an effective amount of a virus inhibitor; or
ii) 0, or a minority of the peptide pools M, NP and S; or E, Pol, C and X, an effective amount of a coronavirus or non-enveloped virus vaccine, respectively.

27. The method of claim 26, wherein the virus is a coronavirus such as a coronavirus selected from the group comprising MERS-CoV, SARS-CoV, SARS-CoV-2, HKU1, OC43, NL63 and 229E or variants thereof; or wherein the virus is HBV or variant thereof.

28. The method of claim 27, wherein the virus is SARS-CoV-2, or HBV.

29. The method of claim 28, wherein the peptide pools comprise M, NP1, NP2 and S pools listed in Tables 1-4; or E1, E2, Pol1, Pol2, Pol3, Pol4, C and X pools listed in Tables 20-27.

30. A method of monitoring the efficacy of a virus vaccine, comprising testing whether the recipient of said vaccine has T cells reactive to 0, a minority, 50%, or a majority of peptide pools derived from the virus M, NP and S proteins; or virus E, Pol, C and X proteins.

31. The method of claim 30, wherein the virus is a coronavirus such as a coronavirus selected from the group comprising MERS-CoV, SARS-CoV, SARS-CoV-2, KHU1, OC43, NL63 and 229E or variants thereof; or HBV or a variant thereof.

32. The method of claim 31, wherein the virus is SARS-CoV-2 or HBV.

33. The method of claim 32, comprising testing whether the recipient of said vaccine has T cells reactive to 0, 1, 2, 3 or 4 of the peptide pools M, NP1, NP2 and S listed in Tables 1-4; or T cells reactive to 0, 1, 2, 3, 4, 5, 6 or 7 of the peptide pools E1, E2, Pol1, Pol2, Pol3, Pol4, C and X.

34. A kit to discriminate past or currently virus-infected subjects from virus un-infected subjects, the kit comprising a plurality of virus structural or non-structural peptides that stimulate virus-exposed T cells, wherein the virus peptides are in peptide pools derived from virus M, NP and S proteins; or from virus E, Pol, C and X proteins.

35. The kit of claim 34, wherein the virus is a coronavirus such as a coronavirus selected from the group comprising MERS-CoV, SARS-CoV, SARS-CoV-2, KHU1, OC43, NL63 and 229E or variants thereof; or HBV or variant thereof.

36. The kit of claim 35, wherein the virus is SARS-CoV-2 or HBV.

37. The kit of claim 36, wherein:
ai) the M protein comprises the amino acid sequence set forth in SEQ ID NO: 793;
ii) the NP protein comprises the amino acid sequence set forth in SEQ ID NO: 794; and
iii) the S protein comprises the amino acid sequence set forth in SEQ ID NO: 795; or
bi) the Pol protein comprises the amino acid sequence set forth in SEQ ID NO: 796;
ii) the E protein comprises the amino acid sequence set forth in SEQ ID NO: 797;
iii) the C protein comprises the amino acid sequence set forth in SEQ ID NO: 798; and
iv) the X protein comprises the amino acid sequence set forth in SEQ ID NO: 799.

38. The kit of claim 37, wherein:
the M peptide pool comprises peptides having amino acid sequences set forth in SEQ ID Nos: 1-43;
the NP peptide pool comprises peptides having amino acid sequences set forth in SEQ ID Nos: 44-125; and
the S peptide pool comprises peptides selected from peptides having amino acid sequences set forth in SEQ ID Nos: 126-454.

39. The kit of claim 34, further comprising one or more reagents to detect cytokines and/or chemokines secreted from activated T cells.

40. A kit to quantify SARS-CoV-2-specific T cell activation, or HBV-specific T cell activation, in an isolated patient sample, comprising:
i) one or more peptide pools selected from the pools in Tables 1-4 and 7-19, or Tables 20-27, respectively;
ii) PCR reagents and/or primers and probes to detect CXCL10 and/or IFN-gamma expression by stimulated T cells; and/or
iii) ELISPOT reagents.

41. The kit of claim 40, wherein peptide pools from Tables 7-19 are used to analyse the effect of virus variants, including variants of concern (VOC), on T cell activation in vaccinated or previously virus-infected subjects.

42. A set of at least 2 separate pools of peptides suitable to discriminate past or currently SARS-CoV-2-infected subjects or vaccinated subjects from SARS-CoV-2 un-infected subjects, wherein the peptide pools are selected from those listed in Tables 1 to 4 and 7-19, or Tables 20-27.
